SEQUENCE LISTING (464 sequences)

SEQ ID NO: 1 - DNA, 384 bp, Synthetic construct
  source 1..384: mol_type = other DNA; note = OZ-A1_HC_nt
caggtgcagctacagcagtggggcgcaggactgttgaagccttcggagaccctgtccctcacctgcgctgtctatggtgggtctttcagtggttactactggagctggatccgccagcccccagggaagggtctggagtggattggggaaatcaatcatagtggattcaccaactacaacccgtccctcaagagtcgagtcaccatatcagtagacacgtccaagaatcacttctccctgaacctgagttctgtgaccgccgcggacacggctgtgtattactgtgcgagaggcgggtattactatggttcggggagttccctctactactactactccggtatggacgtctggggccaagggaccacggtcaccgtctcctca

SEQ ID NO: 2 - protein, 128 aa, Synthetic construct
  source 1..128: note = OZ-A1_HC_aa
QVQLQQWGAGLLKPSETLSLTCAVYGGSFSGYYWSWIRQPPGKGLEWIGEINHSGFTNYNPSLKSRVTISVDTSKNHFSLNLSSVTAADTAVYYCARGGYYYGSGSSLYYYYSGMDVWGQGTTVTVSS

SEQ ID NO: 3 - DNA, 322 bp, Synthetic construct
  source 1..322: mol_type = other DNA; note = OZ-A1_KC_nt
gaaatagtgatgacgcagtctccagccaccctgtctgtgtccccaggggaaagagccaccctctcctgcagggccagtcagagtgtcagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggaaatcaaac

SEQ ID NO: 4 - protein, 107 aa, Synthetic construct
  source 1..107: note = OZ-A1_KC_AA
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 5 - DNA, 363 bp, Synthetic construct
  source 1..363: mol_type = other DNA; note = OZ_B9_HC_nt
aggtgcagctggtggagtctgggggagtcgtggtacagcctggggggtccctgagactgtcctgtgcagtctctggattcacctttgataattctgccatgcactgggtccgtcaagctccggggaagggtctggaatgtgtctctctgattaattgggatggtgataccacatactatgcagactctgtgaagggccgattcaccatctccagagacaacagcagaaactccctgtatctgcaaatgaacagtctgagaactgaggacaccgccttgtattactgtgcaaaagatattggctatggtggctacgggggctttgactactggggccagggaaccctggtcaccgtctcctcag

SEQ ID NO: 6 - protein, 121 aa, Synthetic construct
  source 1..121: note = OZ_B9_HC_AA
EVQLVESGGVVVQPGGSLRLSCAVSGFTFDNSAMHWVRQAPGKGLECVSLINWDGDTTYYADSVKGRFTISRDNSRNSLYLQMNSLRTEDTALYYCAKDIGYGGYGGFDYWGQGTLVTVSS

SEQ ID NO: 7 - DNA, 321 bp, Synthetic construct
  source 1..321: mol_type = other DNA; note = OZ_B9_KC_nt
acatccagttgacccagtctccatccttcctgtctgcatctgtaggagacagagtcaccatcacttgccgggccagtcagggcattagcagttttttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttacccgtacacttttggccaggggaccaagctggagatcaaac

SEQ ID NO: 8 - protein, 107 aa, Synthetic construct
  source 1..107: note = OZ_B9_KC_AA
DIQLTQSPSFLSASVGDRVTITCRASQGISSFLAWYQQKPGKAPKLLIYAASTLQSGVPSRFSGSGSGTEFTLTISSLQPEDFATYYCQQLNSYPYTFGQGTKLEIK

SEQ ID NO: 9 - DNA, 364 bp, Synthetic construct
  source 1..364: mol_type = other DNA; note = OZ_B11_HC_nt
caggtgcagctggtggagtcgggcccaggactggtgaagccttcacagaccctgtccctcacctgcactgtctctggtggctccatcagcagtggtggttactactggagctggatccgccagcacccagggaagggcctggagtggattgggtacatctatcacagtgggagcacctactacaacccgtccctcaagagtcgagttaccatatcagtagacacgtctaagaaccagttctccctgaagctgaggtctgtgactgccgcggacacggccgtgtattactgtgcgagagccaattacgatattttgactgggtttgactactggggccagggaaccctggtcaccgtctcctcag

SEQ ID NO: 10 - protein, 121 aa, Synthetic construct
  source 1..121: note = OZ_B11_HC_AA
QVQLVESGPGLVKPSQTLSLTCTVSGGSISSGGYYWSWIRQHPGKGLEWIGYIYHSGSTYYNPSLKSRVTISVDTSKNQFSLKLRSVTAADTAVYYCARANYDILTGFDYWGQGTLVTVSS

SEQ ID NO: 11 - DNA, 322 bp, Synthetic construct
  source 1..322: mol_type = other DNA; note = OZ_B11_KC_nt
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgttcactttcggcggagggaccaaggtggaaatcaaac

SEQ ID NO: 12 - protein, 107 aa, Synthetic construct
  source 1..107: note = OZ_B11_KC_AA
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGGGTKVEIK

SEQ ID NO: 13 - DNA, 357 bp, Synthetic construct
  source 1..357: mol_type = other DNA; note = OZ_D4_HC_nt
aggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgaggtatagcagtggctggtccttttcttttgatatctggggccaagggacaagggtcaccgtctcttcag

SEQ ID NO: 14 - protein, 119 aa, Synthetic construct
  source 1..119: note = OZ_D4_HC_AA
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARYSSGWSFSFDIWGQGTRVTVSS

SEQ ID NO: 15 - DNA, 331 bp, Synthetic construct
  source 1..331: mol_type = other DNA; note = OZ_D4_LC_nt
cagtctgccctgactcagcctgcctccgtggctgggtctcctggacagtcgatcaccatctcctgcactggaaccagcagtgatgttgggagttataaccttgtctcctggtaccaacaacacccaggcaaagcccccaaactcatgattcatgagggcactaagcggccctcaggggtttctaatcgcttctctggctccaagtctggcaactcggcctccctgacaatctctgggctccaggctgaggacgaggctgattatttctgctcctcatatgcaggtggtaacactttggtgttcggcggagggaccaatctgaccgtcctac

SEQ ID NO: 16 - protein, 110 aa, Synthetic construct
  source 1..110: note = OZ_D4_LC_AA
QSALTQPASVAGSPGQSITISCTGTSSDVGSYNLVSWYQQHPGKAPKLMIHEGTKRPSGVSNRFSGSKSGNSASLTISGLQAEDEADYFCSSYAGGNTLVFGGGTNLTVL

SEQ ID NO: 17 - DNA, 361 bp, Synthetic construct
  source 1..361: mol_type = other DNA; note = OZ_A10_HC_nt
gaggtgcagctggtggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagggtatagcagcagtcgggcttttgatatctggggccaagggacaatggtcaccgtctcttcag

SEQ ID NO: 18 - protein, 120 aa, Synthetic construct
  source 1..120: note = OZ_A10_HC_AA
EVQLVESGPGLVKPSETLSLTCTVSGGSVSSGSYYWSWIRQPPGKGLEWIGYIYYSGSTNYNPSLKSRVTISVDTSKNQFSLKLSSVTAADTAVYYCARGYSSSRAFDIWGQGTMVTVSS

SEQ ID NO: 19 - DNA, 322 bp, Synthetic construct
  source 1..322: mol_type = other DNA; note = OZ_A10_LC_nt
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatttatgctgcatccactttgcaaagtggggtcccatcaaggttcagcggcagtggatttgggacagaattcactctcacaatcagcagcctgcagcctgaagattttgcaacttattactgtcaacagcttaatagttaccctccgaccttcggccaagggacacgactggagattaaac

SEQ ID NO: 20 - protein, 107 aa, Synthetic construct
  source 1..107: note = OZ_A10_LC_AA
DIQLTQSPSFLSASVGDRVTITCRASQGISSYLAWYQQKPGKAPKLLIYAASTLQSGVPSRFSGSGFGTEFTLTISSLQPEDFATYYCQQLNSYPPTFGQGTRLEIK

SEQ ID NO: 21 - DNA, 361 bp, Synthetic construct
  source 1..361: mol_type = other DNA; note = OZ_A3_HC_nt
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagacgaggacacggctgtgtattactgtgcgagagggtattactatggttcggggagtcttgactactggggccagggaaccctggtcaccgtctcctcag

SEQ ID NO: 22 - protein, 120 aa, Synthetic construct
  source 1..120: note = OZ_A3_HC_AA
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCARGYYYGSGSLDYWGQGTLVTVSS

SEQ ID NO: 23 - DNA, 322 bp, synthetic construct
  source 1..322: mol_type = other DNA; note = OZ_A3_LC_nt
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaac

SEQ ID NO: 24 - protein, 107 aa, Synthetic construct
  source 1..107: note = OZ_A3_LC_AA
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 25 - DNA, 354 bp, Synthetic construct
  source 1..354: mol_type = other DNA; note = OZ_A2_HC_nt
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacataactactacggtatggacgtctggggccaagggaccacggtcaccgtctcctca

SEQ ID NO: 26 - protein, 118 aa, Synthetic construct
  source 1..118: note = OZ_A2_HC_AA
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHNYYGMDVWGQGTTVTVSS

SEQ ID NO: 27 - DNA, 328 bp, Synthetic construct
  source 1..328: mol_type = other DNA; note = OZ_A2_LC_nt
gaaatagtgaggaggcactgtccaccccccccgtgtgtgtatccaggggaaagagactccctatcatgcagggccagtcagagggctagcagcaatatatcgcggtgtcagcagaaacatcaccgggatcccaggctcttcatatatggtgcatccaccagggcctctggtatcccagccaggttcagtggcagtgggtatgggacagagttcactctcaccatcagcagcctgcagtgcgaagattttgcagtttattactgtcagcagtataatacagggccccttatgtacacttttggccaggggaccaagctggagatcaaac

SEQ ID NO: 28 - protein, 109 aa, Synthetic construct
  source 1..109: note = OZ_A2_LC_AA
EIVRRHCPPPPCVYPGERDSLSCRASQRASSNISRCQQKHHRDPRLFIYGASTRASGIPARFSGSGYGTEFTLTISSLQCEDFAVYYCQQYNTGPLMYTFGQGTKLEIK

SEQ ID NO: 29 - DNA, 372 bp, Synthetic construct
  source 1..372: mol_type = other DNA; note = OZ_A5_HC_nt
caggtgcagctggtgcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagggatggttcggggagttattataacgtctactactacggtatggacgtctggggccaagggaccacggtcaccgtctcctca

SEQ ID NO: 30 - protein, 124 aa, Synthetic construct
  source 1..124: note = OZ_A5_HC_AA
QVQLVQWGAGLLKPSETLSLTCAVYGGSFSGYYWSWIRQPPGKGLEWIGEINHSGSTNYNPSLKSRVTISVDTSKNQFSLKLSSVTAADTAVYYCARDGSGSYYNVYYYGMDVWGQGTTVTVSS

SEQ ID NO: 31 - DNA, 334 bp, Synthetic construct
  source 1..334: mol_type = other DNA; note = OZ_A5_LC_nt
gatattgtgatgactcagtctccactctccctgcccgtcacccctggagagccggcctccatctcctgcaggtttagtcagagcctccggcatagtaatggatacaactatttggattggtaccggcagaagccagggcagtctccacagctcctgatctatttgggttttaatcgggcctccggggtccctgacaggttcagcggcagcggatcaggcacagattttacactgaaaatcagcagaggggaggcggaggatgttggggtttattactgcacgcaagctctacaaactctcactttcggcggagggaccaaggtggagatcaaac

SEQ ID NO: 32 - protein, 111 aa, Synthetic construct
  source 1..111: note = OZ_A5_LC_AA
DIVMTQSPLSLPVTPGEPASISCRFSQSLRHSNGYNYLDWYRQKPGQSPQLLIYLGFNRASGVPDRFSGSGSGTDFTLKISRGEAEDVGVYYCTQALQTLTFGGGTKVEIK

SEQ ID NO: 33 - DNA, 381 bp, Synthetic construct
  source 1..381: mol_type = other DNA; note = OZ_A6_HC_nt
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtggaagcaccaactacaacccgtccctcaagagtcgagtcaccatatcagtagacaagtccaagaaccagttctccctgaagctgagctctgtgaccgccgcggacacggccgtgtattactgtgcgagaggccaattactatggttcggggagttggggtactactactacggtatggacgtctggggccaagggaccacggtcaccgtctcctca

SEQ ID NO: 34 - protein, 127 aa, Synthetic construct
  source 1..127: note = OZ_A6_HC_AA
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGQLLWFGELGYYYYGMDVWGQGTTVTVSS

SEQ ID NO: 35 - DNA, 322 bp, Synthetic construct
  source 1..322: mol_type = other DNA; note = OZ_A6_LC_nt
gacatccagatgacccagtctccatcctccctgtctgcatctgtaggagacagagtcaccatcacttgccgggcaagtcggggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtttacagcataatagttaccctcggacgttcggccaagggaccaaggtggaaatcaaac

SEQ ID NO: 36 - protein, 107 aa, Synthetic construct
  source 1..107: note = OZ_A6_LC_AA
DIQMTQSPSSLSASVGDRVTITCRASRGIRNDLGWYQQKPGKAPKRLIYAASSLQSGVPSRFSGSGSGTEFTLTISSLQPEDFATYYCLQHNSYPRTFGQGTKVEIK

SEQ ID NO: 37 - DNA, 364 bp, synthetic construct
  source 1..364: mol_type = other DNA; note = OZ_A9B_HC_nt
caggtgcagctggtggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagctatggttcggggagttattataactggtacttcgatctctggggccgtggcaccctggtcaccgtctcctcag

SEQ ID NO: 38 - protein, 121 aa, Synthetic construct
  source 1..121: note = OZ_A9B_HC_AA
QVQLVESGPGLVKPSETLSLTCTVSGGSISSYYWSWIRQPPGKGLEWIGYIYYSGSTNYNPSLKSRVTISVDTSKNQFSLKLSSVTAADTAVYYCASYGSGSYYNWYFDLWGRGTLVTVSS

SEQ ID NO: 39 - DNA, 322 bp, Synthetic construct
  source 1..322: mol_type = other DNA; note = OZ_A9B_LC_nt
gaaattgtgctgactcagtctccagactttcagtctgtgactccaaaggagaaagtcaccatcacctgccgggccagtcagaccattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcatcagagtagtagtttacctcacacttttggccaggggaccaagctggagatcaaac

SEQ ID NO: 40 - protein, 107 aa, Synthetic construct
  source 1..107: note = OZ_A9B_LC_AA
EIVLTQSPDFQSVTPKEKVTITCRASQTIGSSLHWYQQKPDQSPKLLIKYASQSFSGVPSRFSGSGSGTDFTLTINSLEAEDAATYYCHQSSSLPHTFGQGTKLEIK

SEQ ID NO: 41 - DNA, 366 bp, Synthetic construct
  source 1..366: mol_type = other DNA; note = OZ_A9C_HC_nt
caggtgcagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataagtactatgcagactccgtgaagggccgattcaccatctccagagacaattccaagaacacgctctatctgcaaatgaacagcctgagagctgaggacacggctgtgttttactgtgcgaaggatcaagactacggtgcctccccccacggtatggacgtctggggccaagggaccacggtcaccgtctcctca

SEQ ID NO: 42 - protein, 122 aa, Synthetic construct
  source 1..122: note = OZ_A9C_HC_AA
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVFYCAKDQDYGASPHGMDVWGQGTTVTVSS

SEQ ID NO: 43 - DNA, 337 bp, Synthetic construct
  source 1..337: mol_type = other DNA; note = OZ_A9C_LC_nt
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggcctcggacgttcggccaagggaccaaggtggaaatcaaac

SEQ ID NO: 44 - protein, 112 aa, Synthetic construct
  source 1..112: note = OZ_A9C_LC_AA
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPRTFGQGTKVEIK

SEQ ID NO: 45 - DNA, 358 bp, Synthetic construct
  source 1..358: mol_type = other DNA; note = OZ_B4_HC_nt
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaccccgctggggttctgatgcttttgatatctggggccaagggacaatggtcaccgtctcttcag

SEQ ID NO: 46 - protein, 119 aa, synthetic construct
  source 1..119: note = OZ_B4_HC_AA
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKPRWGSDAFDIWGQGTMVTVSS

SEQ ID NO: 47 - DNA, 331 bp, Synthetic construct
  source 1..331: mol_type = other DNA; note = OZ_B4_LC_nt
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacttgggtgttcggcggagggaccaagctgaccgtcctag

SEQ ID NO: 48 - protein, 110 aa, Synthetic construct
  source 1..110: note = OZ_B4_LC_AA
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTWVFGGGTKLTVL

SEQ ID NO: 49 - DNA, 376 bp, Synthetic construct
  source 1..376: mol_type = other DNA; note = OZ_B10_HC_nt
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagatattattactatggttcggggagttattataacctactactttgactactggggccagggaaccctggtcactgtctcctcag

SEQ ID NO: 50 - protein, 125 aa, Synthetic construct
  source 1..125: note = OZ_B10_HC_AA
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDIITMVRGVIITYYFDYWGQGTLVTVSS

SEQ ID NO: 51 - DNA, 325 bp, Synthetic construct
  source 1..325: mol_type = other DNA; note = OZ_B10_LC_nt
tcctatgagctgacacagccaccctcggtgtcagtgtccccaggacaaacggccaggatcacctgttttggagatgcattgccaaaaaaatatgcttattggtaccagcagaagtcaggccaggcccctgtgctggtcatctatgaggacagcaaacgaccctccgggatccctgagagattctctggctccagctcagggacaatggccaccttgactatcagtggggcccaggtggaggatgaagctgactactactgttactcaacagacagcagtggtaatcatagggtattcggcggagggaccaagctgaccgtcctag

SEQ ID NO: 52 - protein, 108 aa, Synthetic construct
  source 1..108: note = OZ_B10_LC_AA
SYELTQPPSVSVSPGQTARITCFGDALPKKYAYWYQQKSGQAPVLVIYEDSKRPSGIPERFSGSSSGTMATLTISGAQVEDEADYYCYSTDSSGNHRVFGGGTKLTVL

SEQ ID NO: 53 - DNA, 367 bp, Synthetic construct
  source 1..367: mol_type = other DNA; note = OZ_C4_HC_nt
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatggcggggttcggggagttcctgatgcttttgatatctggggccaagggacaatggtcaccgtctcttcag

SEQ ID NO: 54 - protein, 122 aa, Synthetic construct
  source 1..122: note = OZ_C4_HC_AA
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGVRGVPDAFDIWGQGTMVTVSS

SEQ ID NO: 55 - DNA, 325 bp, Synthetic construct
  source 1..325: mol_type = other DNA; note = OZ_C4_LC_nt
tcctatgagctgacacagccaccctcggtgtcagtgtccccaggacaaacggccaggatcacctgctctggagatgcattgccaaaaaaatatgcttattggtaccagcagaagtcaggccaggcccctgtgctggtcatctatgaggacagcaaacgaccctccgggatccctgagagattctctggctccagctcagggacaatggccaccttgactatcagtggggcccaggtggaggatgaagctgactactactgttactcaacagacagcagtggtaatcatagtgtattcggcggagggaccaagctgaccgtcctag

SEQ ID NO: 56 - protein, 108 aa, Synthetic construct
  source 1..108: note = OZ_C4_LC_AA
SYELTQPPSVSVSPGQTARITCSGDALPKKYAYWYQQKSGQAPVLVIYEDSKRPSGIPERFSGSSSGTMATLTISGAQVEDEADYYCYSTDSSGNHSVFGGGTKLTVL

SEQ ID NO: 57 - DNA, 372 bp, Synthetic construct
  source 1..372: mol_type = other DNA; note = OZ_C7_HC_nt
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatgatgggagctactcttactactactactacggtatggacgtctggggccaagggaccacggtcaccgtctcctca

SEQ ID NO: 58 - protein, 124 aa, Synthetic construct
  source 1..124: note = OZ_C7_HC_AA
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDDGSYSYYYYYGMDVWGQGTTVTVSS

SEQ ID NO: 59 - DNA, 325 bp, Synthetic construct
  source 1..325: mol_type = other DNA; note = OZ_C7_LC_nt
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcattggggctcaggcggaagatgaggctgactattactgtaactcccgggacagcagtggtaaccattgggtgttcggcggagggaccaagctgaccgtcctag

SEQ ID NO: 60 - protein, 108 aa, Synthetic construct
  source 1..108: note = OZ_C7_LC_AA
SSELTQDPAVSVALGQTVRITCQGDSLRSYYASWYQQKPGQAPVLVIYGKNNRPSGIPDRFSGSSSGNTASLTIIGAQAEDEADYYCNSRDSSGNHWVFGGGTKLTVL

SEQ ID NO: 61 - DNA, 367 bp, synthetic construct
  source 1..367: mol_type = other DNA; note = OZ_C8_HC_nt
caggtgcagctggtggagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagacttactatggttcggggagcccgatgcttttgatatctggggccaagggacaatggtcaccgtctcttcag

SEQ ID NO: 62 - protein, 122 aa, Synthetic construct
  source 1..122: note = OZ_C8_HC_AA
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCARDLLWFGEPDAFDIWGQGTMVTVSS

SEQ ID NO: 63 - DNA, 316 bp, Synthetic construct
  source 1..316: mol_type = other DNA; note = OZ_C8_LC_nt
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagtttcactttcggcggagggaccaaggtggagatcaaac

SEQ ID NO: 64 - protein, 105 aa, Synthetic construct
  source 1..105: note = OZ_C8_LC_AA
DIQMTQSPSTLSASVGDRVTITCRASQSISSWLAWYQQKPGKAPKLLIYKASSLESGVPSRFSGSGSGTEFTLTISSLQPDDFATYYCQQYNSFTFGGGTKVEIK

SEQ ID NO: 65 - DNA, 367 bp, Synthetic construct
  source 1..367: ecotype = OZ_D2_HC_nt; mol_type = other DNA
gaggtgcagctggtggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagatcgttatagcagcagctggtacccttttgactactggggccagggaaccctggtcaccgtctcctcag

SEQ ID NO: 66 - protein, 122 aa, Synthetic construct
  source 1..122: note = OZ_D2_HC_AA
EVQLVESGPGLVKPSETLSLTCTVSGGSVSSGSYYWSWIRQPPGKGLEWIGYIYYSGSTNYNPSLKSRVTISVDTSKNQFSLKLSSVTAADTAVYYCARDRYSSSWYPFDYWGQGTLVTVSS

SEQ ID NO: 67 - DNA, 337 bp, synthetic construct
  source 1..337: mol_type = other DNA; note = OZ_D2_LC_nt
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaaatcaaac

SEQ ID NO: 68 - protein, 112 aa, Synthetic construct
  source 1..112: note = OZ_D2_LC_AA
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIK

SEQ ID NO: 69 - DNA, 373 bp, Synthetic construct
  source 1..373: mol_type = other DNA; note = OZ_D6_HC_nt
gaggtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatcattacgatattttgactggttattataccccctttgactactggggccagggaaccctggtcaccgtctcctcag

SEQ ID NO: 70 - protein, 124 aa, Synthetic construct
  source 1..124: note = OZ_D6_HC_AA
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDHYDILTGYYTPFDYWGQGTLVTVSS

SEQ ID NO: 71 - DNA, 322 bp, Synthetic construct; identical to SEQ ID NO: 35
  source 1..322: mol_type = other DNA; note = OZ_D6_LC_nt

SEQ ID NO: 72 - protein, 107 aa, Synthetic construct; identical to SEQ ID NO: 36
  source 1..107: note = OZ_D6_LC_AA

SEQ ID NO: 73 - DNA, 358 bp, Synthetic construct
  source 1..358: mol_type = other DNA; note = OZ_D9_HC_nt
caggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagggccaaactggggattgctttttgactactggggccagggaaccctggtcaccgtctcctcag

SEQ ID NO: 74 - protein, 119 aa, Synthetic construct
  source 1..119: note = OZ_D9_HC_AA
QVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGPNWGLLFDYWGQGTLVTVSS

SEQ ID NO: 75 - DNA, 319 bp, Synthetic construct
  source 1..319: mol_type = other DNA; note = OZ_D9_LC_nt
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcactgtggtattcggcggagggaccaagctgaccgtcctag

SEQ ID NO: 76 - protein, 106 aa, Synthetic construct
  source 1..106: note = OZ_D9_LC_AA
SYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 77 - DNA, 367 bp, synthetic construct
  source 1..367: mol_type = other DNA; note = OZ_E3_HC_nt
caggtgcagctggtggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaggtatagcagtggctctctactggtacttcgatctctggggccgtggcaccctggtcaccgtctcctcag

SEQ ID NO: 78 - protein, 122 aa, Synthetic construct
  source 1..122: note = OZ_E3_HC_AA
QVQLVESGPGLVKPSETLSLTCTVSGGSVSSGSYYWSWIRQPPGKGLEWIGYIYYSGSTNYNPSLKSRVTISVDTSKNQFSLKLSSVTAADTAVYYCARGIAVALYWYFDLWGRGTLVTVSS

SEQ ID NO: 79 - DNA, 319 bp, Synthetic construct; identical to SEQ ID NO: 75
  source 1..319: mol_type = other DNA; note = OZ_E3_LC_nt

SEQ ID NO: 80 - protein, 106 aa, Synthetic construct; identical to SEQ ID NO: 76
  source 1..106: note = OZ_E3_LC_AA

SEQ ID NO: 81 - DNA, 373 bp, synthetic construct
  source 1..373: mol_type = other DNA; note = OZ_F2_HC_nt
gaggtgcagctggaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagaccctattactatggttcggggagtcttgatgcttttgatatctggggccaagggacaatggtcaccgtctcttcag

SEQ ID NO: 82 - protein, 124 aa, Synthetic construct
  source 1..124: note = OZ_F2_HC_AA
EVQLEESGPGLVKPSETLSLTCTVSGGSISSSSYYWGWIRQPPGKGLEWIGSIYYSGSTYYNPSLKSRVTISVDTSKNQFSLKLSSVTAADTAVYYCARPYYYGSGSLDAFDIWGQGTMVTVSS

SEQ ID NO: 83 - DNA, 319 bp, Synthetic construct
  source 1..319: mol_type = other DNA; note = OZ_F2_LC_nt
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcggacgttcggccaagggaccaaggtggaaatcaaac

SEQ ID NO: 84 - protein, 106 aa, Synthetic construct
  source 1..106: note = OZ_F2_LC_AA
EIVMTQSPATLSVSPGERATLSCRASQSVSSNLAWYQQKPGQAPRLLIYGASTRATGIPARFSGSGSGTEFTLTISSLQSEDFAVYYCQQYNNWRTFGQGTKVEIK

SEQ ID NO: 85 - DNA, 355 bp, Synthetic construct
  source 1..355: mol_type = other DNA; note = OZ_F3_HC_nt
gaggtgcagctggtggagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacatcaagtgggagcctactttgactactggggccagggaaccctggtcaccgtctcctcag

SEQ ID NO: 86 - protein, 118 aa, Synthetic construct
  source 1..118: note = OZ_F3_HC_AA
EVQLVESGAEVKKPGESLKISCKGSGYSFTSYWIGWVRQMPGKGLEWMGIIYPGDSDTRYSPSFQGQVTISADKSISTAYLQWSSLKASDTAMYYCARHQVGAYFDYWGQGTLVTVSS

SEQ ID NO: 87 - DNA, 331 bp, Synthetic construct
  source 1..331: mol_type = other DNA; note = OZ_F3_LC_nt
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaacatttgggtgttcggcggagggaccaagctgaccgtcctag

SEQ ID NO: 88 - protein, 110 aa, Synthetic construct
  source 1..110: note = OZ_F3_LC_AA
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNIWVFGGGTKLTVL

SEQ ID NO: 89 - DNA, 391 bp, synthetic construct
  source 1..391: mol_type = other DNA; note = OZ_H2_HC_nt
gaggtgcagctggtggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacaatggggtctaattactatggttcggggagttattaaaacggtgaactggttcgacccctggggccagggaaccctggtcaccgtctcctcag

SEQ ID NO: 90 - protein, 130 aa, Synthetic construct
  source 1..130: note = OZ_H2_HC_AA
EVQLVESGPGLVKPSETLSLTCTVSGGSISSSSYYWGWIRQPPGKGLEWIGSIYYSGSTYYNPSLKSRVTISVDTSKNQFSLKLSSVTAADTAVYYCARQWGLITMVRGVIKTVNWFDPWGQGTLVTVSS

SEQ ID NO: 91 - DNA, 142 bp, Synthetic construct
  source 1..142: mol_type = other DNA; note = OZ_H2_LC_nt
cgattcagtggcagcgggtctgggacagatttcactctcaccatcagcagcctgcaggctgaagatgtggcagtttattactgtcagcaatattatagtactcctccgacgttcggccaagggaccaaggtggaaatcaaac

SEQ ID NO: 92 - protein, 47 aa, Synthetic construct
  source 1..47: note = OZ_H2_LC_AA
RFSGSGSGTDFTLTISSLQAEDVAVYYCQQYYSTPPTFGQGTKVEIK

SEQ ID NO: 93 - DNA, 373 bp, Synthetic construct
  source 1..373: mol_type = other DNA; note = OZ_H6_HC_nt
gaggtgcagctggtggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagaacgtattactatggttcggggattcatccctgatgcttttgatatctggggccaagggacaatggtcaccgtctcttcag

SEQ ID NO: 94 - protein, 124 aa, Synthetic construct
  source 1..124: note = OZ_H6_HC_AA
EVQLVESGPGLVKPSETLSLTCTVSGGSISSYYWSWIRQPPGKGLEWIGYIYYSGSTNYNPSLKSRVTISVDTSKNQFSLKLSSVTAADTAVYYCARERITMVRGFIPDAFDIWGQGTMVTVSS

SEQ ID NO: 95 - DNA, 325 bp, Synthetic construct
  source 1..325: mol_type = other DNA; note = OZ_H6_LC_nt
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacgttcggccaagggaccaaggtggaaatcaaac

SEQ ID NO: 96 - protein, 108 aa, Synthetic construct
  source 1..108: note = OZ_H6_LC_AA
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 97 - DNA, 355 bp, Synthetic construct
  source 1..355: mol_type = other DNA; note = OZ_H8_HC_nt
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagatatggggatcggtgcttttgatatctggggccaagggacaatggtcaccgtctcttcag

SEQ ID NO: 98 - protein, 118 aa, Synthetic construct
  source 1..118: note = OZ_H8_HC_AA
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARDMGIGAFDIWGQGTMVTVSS

SEQ ID NO: 99 - DNA, 331 bp, Synthetic construct
  source 1..331: mol_type = other DNA; note = OZ_H8_LC_nt
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaacaatttggtattcggcggagggaccaagctgaccgtcctag

SEQ ID NO: 100 - protein, 110 aa, Synthetic construct
  source 1..110: note = OZ_H8_LC_AA
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKLTVL

SEQ ID NO: 101 - DNA, 370 bp, Synthetic construct
  source 1..370: mol_type = other DNA; note = OZ_H10_HC_nt
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggaggtactatggttcggggagttacttcgtactttgactactggggccagggaaccctggtcaccgtctcctcag

SEQ ID NO: 102 - protein, 123 aa, Synthetic construct
  source 1..123: note = OZ_H10_HC_AA
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGTMVRGVTSYFDYWGQGTLVTVSS

SEQ ID NO: 103 - DNA, 325 bp, Synthetic construct
  source 1..325: mol_type = other DNA; note = OZ_H10_LC_nt
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatctggtgttcggcggagggaccaagctgaccgtcctag

SEQ ID NO: 104 - protein, 108 aa, synthetic construct
  source 1..108: note = OZ_H10_LC_AA
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTKLTVL

SEQ ID NO: 105 - DNA, 361 bp, Synthetic construct
  source 1..361: mol_type = other DNA; note = OZ_A4_HC_nt
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagttatggttcggggagttataacctctttgactactggggccagggaaccctggtcaccgtctcctcag

SEQ ID NO: 106 - protein, 120 aa, Synthetic construct
  source 1..120: note = OZ_A4_HC_AA
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCASYGSGSYNLFDYWGQGTLVTVSS

SEQ ID NO: 107 - DNA, 325 bp, Synthetic construct
  source 1..325: mol_type = other DNA; note = OZ_A4_LC_nt
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtacacttttggccaggggaccaagctggagatcaaac

SEQ ID NO: 108 - protein, 108 aa, Synthetic construct
  source 1..108: note = OZ_A4_LC_AA
EIVLTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTKLEIK

SEQ ID NO: 109-110 - skipped (2 entries)

SEQ ID NO: 111 - DNA, 325 bp, Synthetic construct
  source 1..325: mol_type = other DNA; note = OZ_E11K_LC_nt
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattctccgtacacttttggccaggggaccaagctggagatcaaac

SEQ ID NO: 112 - protein, 108 aa, Synthetic construct
  source 1..108: note = OZ_E11K_LC_AA
DIQMTQSPSTLSASVGDRVTITCRASQSISSWLAWYQQKPGKAPKLLIYKASSLESGVPSRFSGSGSGTEFTLTISSLQPDDFATYYCQQYNSYSPYTFGQGTKLEIK

SEQ ID NO: 113 - DNA, 364 bp, Synthetic construct
  source 1..364: mol_type = other DNA; note = OZ_E11L_HC_nt
caggtgcagctggtggagtctgggggaggcttggtccagcctggggggtccctgagactctcctgtgcagcctctggattcacctttagtaactattggatgagctgggtccgccaggctccagggaaggggctggagtgggtggccaacataaagcaagatggaagtgacaaatactatgtggactctgtgaagggccgattcaccatctccagagacaacgccaaaaactcactgtatctgcaaatgaacagcctgagagccgaggacacggctgtgtattactgtgcgagagaatatagcagcagctggtactcgacctttgactactggggccagggaaccctggtcaccgtctcctcag

SEQ ID NO: 114 - protein, 121 aa, Synthetic construct
  source 1..121: note = OZ_E11L_HC_AA
QVQLVESGGGLVQPGGSLRLSCAASGFTFSNYWMSWVRQAPGKGLEWVANIKQDGSDKYYVDSVKGRFTISRDNAKNSLYLQMNSLRAEDTAVYYCAREYSSSWYSTFDYWGQGTLVTVSS

SEQ ID NO: 115 - DNA, 325 bp, Synthetic construct
  source 1..325: mol_type = other DNA; note = OZ_E11L_LC_nt
tcctatgtgctgactcagccaccctcggtgtcagtggccccaggacagacggccaggattacctgtgggggaaacaacattggaagtacaagtgtgcactggtaccagcagaagccaggccaggcccctgtgctggtcgtctatgatgatcgtgaccggccctcagggatccctgagcgattctctggctccaattctgggaacacggccaccctgaccatcagtagggtcgaagccggggatgaggccgactattactgtcaggtgtgggatagtagtagtgatcatgtggtattcggcggagggaccaagctgaccgtcctag

SEQ ID NO: 116 - protein, 108 aa, Synthetic construct
  source 1..108: note = OZ_E11L_LC_AA
SYVLTQPPSVSVAPGQTARITCGGNNIGSTSVHWYQQKPGQAPVLVVYDDRDRPSGIPERFSGSNSGNTATLTISRVEAGDEADYYCQVWDSSSDHVVFGGGTKLTVL

SEQ ID NO: 117 - protein, 8 aa, Synthetic construct
  source 1..8: note = OZ-A1 HCDR1
GGSFSGYY

SEQ ID NO: 118 - protein, 7 aa, Synthetic construct
  source 1..7: note = OZ-A1 HCDR2
INHSGFT

SEQ ID NO: 119 - protein, 22 aa, Synthetic construct
  source 1..22: note = OZ-A1 HCDR3
ARGGYYYGSGSSLYYYYSGMDV

SEQ ID NO: 120 - protein, 10 aa, Synthetic construct
  source 1..10: note = OZ-A10 HCDR1
GGSVSSGSYY

SEQ ID NO: 121 - protein, 7 aa, Synthetic construct
  source 1..7: note = OZ-A10 HCDR2
IYYSGST

SEQ ID NO: 122 - protein, 12 aa, synthetic construct
  source 1..12: note = OZ_A10_HCDR3
ARGYSSSRAFDI

SEQ ID NO: 123 - protein, 8 aa, Synthetic construct
  source 1..8: note = OZ-A3 HCDR1
GFTFSSYS

SEQ ID NO: 124 - protein, 8 aa, Synthetic construct
  source 1..8: note = OZ-A3 HCDR2
ISSSSSTI

SEQ ID NO: 125 - protein, 13 aa, Synthetic construct
  source 1..13: note = OZ-A3 HCDR3
ARGYYYGSGSLDY

SEQ ID NO: 126 - protein, 10 aa, Synthetic construct
  source 1..10: note = OZ-A2 HCDR1
GGSISSSSYY

SEQ ID NO: 127 - protein, 7 aa, Synthetic construct
  source 1..7: note = OZ-A2 HCDR2
IYYSGST

SEQ ID NO: 128 - protein, 10 aa, Synthetic construct
  source 1..10: note = OZ-A2 HCDR3
ARHNYYGMDV

SEQ ID NO: 129 - protein, 8 aa, Synthetic construct
  source 1..8: note = OZ-A5 HCDR1
GGSFSGYY

SEQ ID NO: 130 - protein, 7 aa, Synthetic construct
  source 1..7: note = OZ-A5 HCDR2
INHSGST

SEQ ID NO: 131 - protein, 18 aa, synthetic construct
  source 1..18: note = OZ_A5_HCDR3
ARDGSGSYYNVYYYGMDV

SEQ ID NO: 132 - protein, 9 aa, Synthetic construct
  source 1..9: note = OZ-A6 HCDR1
GGSISSSNW

SEQ ID NO: 133 - protein, 7 aa, Synthetic construct
  source 1..7: note = OZ-A6 HCDR2
IYHSGST

SEQ ID NO: 134 - protein, 20 aa, synthetic construct
  source 1..20: note = OZ_A6_HCDR3
ARGQLLWFGELGYYYYGMDV

SEQ ID NO: 135 - protein, 8 aa, Synthetic construct
  source 1..8: note = OZ-A9B HCDR1
GGSISSYY

SEQ ID NO: 136 - protein, 7 aa, Synthetic construct
  source 1..7: note = OZ-A9B HCDR2
IYYSGST

SEQ ID NO: 137 - protein, 15 aa, Synthetic construct
  source 1..15: note = OZ-A9B HCDR3
ASYGSGSYYNWYFDL

SEQ ID NO: 138 - protein, 8 aa, Synthetic construct
  source 1..8: note = OZ-A9C HCDR1
GFTFSSYG

SEQ ID NO: 139 - protein, 8 aa, Synthetic construct
  source 1..8: note = OZ-A9C HCDR2
ISYDGSNK

SEQ ID NO: 140 - protein, 15 aa, Synthetic construct
  source 1..15: note = OZ-A9C HCDR3
AKDQDYGASPHGMDV

SEQ ID NO: 141 - protein, 8 aa, Synthetic construct
  source 1..8: note = OZ-B4 HCDR1
GFTFSSYG

SEQ ID NO: 142 - protein, 8 aa, Synthetic construct
  source 1..8: note = OZ-B4 HCDR2
ISYDGSNK

SEQ ID NO: 143 - protein, 12 aa, Synthetic construct
  source 1..12: note = OZ-B4 HCDR3
AKPRWGSDAFDI

SEQ ID NO: 144 - protein, 8 aa, Synthetic construct
  source 1..8: note = OZ-B10 HCDR1
GFTVSSNY

SEQ ID NO: 145 - protein, 7 aa, Synthetic construct
  source 1..7: note = OZ-B10 HCDR2
IYSGGST

SEQ ID NO: 146 - protein, 19 aa, Synthetic construct
  source 1..19: note = OZ-B10 HCDR3
ARDIITMVRGVIITYYFDY

SEQ ID NO: 147 - protein, 10 aa, Synthetic construct
  source 1..10: note = OZ-B11 HCDR1
GGSISSGGYY

SEQ ID NO: 148 - protein, 7 aa, Synthetic construct
  source 1..7: note = OZ-B11 HCDR2
IYHSGST

SEQ ID NO: 149 - protein, 13 aa, Synthetic construct
  source 1..13: note = OZ-B11 HCDR3
ARANYDILTGFDY

SEQ ID NO: 150 - protein, 8 aa, Synthetic construct
  source 1..8: note = OZ-C4 HCDR1
GFTFSSYA

SEQ ID NO: 151 - protein, 8 aa, Synthetic construct
  source 1..8: note = OZ-C4 HCDR2
ISGSGGST

SEQ ID NO: 152 - protein, 15 aa, Synthetic construct
  source 1..15: note = OZ-C4 HCDR3
AKDGGVRGVPDAFDI

SEQ ID NO: 153 - protein, 8 aa, Synthetic construct
  source 1..8: note = OZ-C7 HCDR1
GFTFSSYS

SEQ ID NO: 154 - protein, 8 aa, Synthetic construct
  source 1..8: note = OZ-C7 HCDR2
ISSSSSYI

SEQ ID NO: 155 - protein, 17 aa, Synthetic construct
  source 1..17: note = OZ-C7 HCDR3
ARDDGSYSYYYYYGMDV

SEQ ID NO: 156 - protein, 8 aa, Synthetic construct
  source 1..8: note = OZ-C8 HCDR1
GYTFTGYY

SEQ ID NO: 157 - protein, 8 aa, Synthetic construct
  source 1..8: note = OZ-C8 HCDR2
INPNSGGT

SEQ ID NO: 158 - protein, 15 aa, Synthetic construct
  source 1..15: note = OZ-C8 HCDR3
ARDLLWFGEPDAFDI

SEQ ID NO: 159 - protein, 10 aa, Synthetic construct
  source 1..10: note = OZ-D2 HCDR1
GGSVSSGSYY

SEQ ID NO: 160 - protein, 7 aa, Synthetic construct
  source 1..7: note = OZ-D2 HCDR2
IYYSGST

SEQ ID NO: 161 - protein, 14 aa, Synthetic construct
  source 1..14: note = OZ-D2 HCDR3
ARDRYSSSWYPFDY

SEQ ID NO: 162 - protein, 8 aa, Synthetic construct
  source 1..8: note = OZ-D4 HCDR1
GGSISSYY

SEQ ID NO: 163 - protein, 7 aa, Synthetic construct
  source 1..7: note = OZ-D4 HCDR2
IYYSGST

SEQ ID NO: 164 - protein, 13 aa, Synthetic construct
  source 1..13: note = OZ-D4 HCDR3
ARYSSGWSFSFDI

SEQ ID NO: 165 - protein, 8 aa, Synthetic construct
  source 1..8: note = OZ-D6 HCDR1
GFTFDDYA

SEQ ID NO: 166 - protein, 8 aa, Synthetic construct
  source 1..8: note = OZ-D6 HCDR2
ISWNSGSI

SEQ ID NO: 167 - protein, 17 aa, synthetic construct
  source 1..17: note = OZ_D6_HCDR3
AKDHYDILTGYYTPFDY

SEQ ID NO: 168 - protein, 8 aa, Synthetic construct
  source 1..8: note = OZ-D9 HCDR1
GFTFSSYW

SEQ ID NO: 169 - protein, 8 aa, Synthetic construct
  source 1..8: note = OZ-D9 HCDR2
IKQDGSEK

SEQ ID NO: 170 - protein, 12 aa, Synthetic construct
  source 1..12: note = OZ-D9 HCDR3
ARGPNWGLLFDY

SEQ ID NO: 171 - protein, 10 aa, synthetic construct
  source 1..10: note = OZ_E3_HCDR1
GGSVSSGSYY

SEQ ID NO: 172 - protein, 7 aa, Synthetic construct
  source 1..7: note = OZ-E3 HCDR2
IYYSGST

SEQ ID NO: 173 - protein, 14 aa, Synthetic construct
  source 1..14: note = OZ-E3 HCDR3
ARGIAVALYWYFDL

SEQ ID NO: 174 - protein, 10 aa, synthetic construct
  source 1..10: note = OZ_F2_HCDR1
GGSISSSSYY

SEQ ID NO: 175 - protein, 7 aa, Synthetic construct
  source 1..7: note = OZ-F2 HCDR2
IYYSGST

SEQ ID NO: 176 - protein, 16 aa, Synthetic construct
  source 1..16: note = OZ-F2 HCDR3
ARPYYYGSGSLDAFDI

SEQ ID NO: 177 - protein, 8 aa, Synthetic construct
  source 1..8: note = OZ-F3 HCDR1
GYSFTSYW

SEQ ID NO: 178 - protein, 8 aa, Synthetic construct
  source 1..8: note = OZ-F3 HCDR2
IYPGDSDT

SEQ ID NO: 179 - protein, 11 aa, Synthetic construct
  source 1..11: note = OZ-F3 HCDR3
ARHQVGAYFDY

SEQ ID NO: 180 - protein, 10 aa, Synthetic construct
  source 1..10: note = OZ-H2 HCDR1
GGSISSSSYY

SEQ ID NO: 181 - protein, 7 aa, Synthetic construct
  source 1..7: note = OZ-H2 HCDR2
IYYSGST

SEQ ID NO: 182 - protein, 22 aa, Synthetic construct
  source 1..22: note = OZ-H2 HCDR3
ARQWGLITMVRGVIKTVNWFDP

SEQ ID NO: 183 - protein, 8 aa, Synthetic construct
  source 1..8: note = OZ-H6 HCDR1
GGSISSYY

SEQ ID NO: 184 - protein, 7 aa, Synthetic construct
  source 1..7: note = OZ-H6 HCDR2
IYYSGST

SEQ ID NO: 185 - protein, 18 aa, Synthetic construct
  source 1..18: note = OZ-H6 HCDR3
ARERITMVRGFIPDAFDI

SEQ ID NO: 186 - protein, 8 aa, Synthetic construct
  source 1..8: note = OZ-H8 HCDR1
GYTFTGYY

SEQ ID NO: 187 - protein, 8 aa, Synthetic construct
  source 1..8: note = OZ-H8 HCDR2
INPNSGGT

SEQ ID NO: 188 - protein, 11 aa, Synthetic construct
  source 1..11: note = OZ-H8 HCDR3
ARDMGIGAFDI

SEQ ID NO: 189 - protein, 8 aa, Synthetic construct
  source 1..8: note = OZ-H10 HCDR1
GFTFSSYA

SEQ ID NO: 190 - protein, 8 aa, Synthetic construct
  source 1..8: note = OZ-H10 HCDR2
ISGSGGST

SEQ ID NO: 191 - protein, 16 aa, synthetic construct
  source 1..16: note = OZ_H10_HCDR3
AKGGTMVRGVTSYFDY

SEQ ID NO: 192 - protein, 8 aa, Synthetic construct
  source 1..8: note = OZ-A4 HCDR1
GFTFSSYS

SEQ ID NO: 193 - protein, 8 aa, Synthetic construct
  source 1..8: note = OZ-A4 HCDR2
ISSSSSYI

SEQ ID NO: 194 - protein, 13 aa, Synthetic construct
  source 1..13: note = OZ-A4 HCDR3
ASYGSGSYNLFDY

SEQ ID NO: 195 - protein, 8 aa, Synthetic construct
  source 1..8: note = OZ-B9 HCDR1
GFTFDNSA

SEQ ID NO: 196 - protein, 8 aa, Synthetic construct
  source 1..8: note = OZ-B9 HCDR2
INWDGDTT

SEQ ID NO: 197 - protein, 14 aa, Synthetic construct
  source 1..14: note = OZ-B9 HCDR3
AKDIGYGGYGGFDY

SEQ ID NO: 198-200 - skipped (3 entries)

SEQ ID NO: 201 - protein, 8 aa, Synthetic construct
  source 1..8: note = OZ-E11L HCDR1
GFTFSNYW

SEQ ID NO: 202 - protein, 8 aa, Synthetic construct
  source 1..8: note = OZ-E11L HCDR2
IKQDGSDK

SEQ ID NO: 203 - protein, 14 aa, Synthetic construct
  source 1..14: note = OZ-E11L HCDR3
AREYSSSWYSTFDY

SEQ ID NO: 204 - protein, 6 aa, Synthetic construct
  source 1..6: note = OZ-A1 LCR1
QSVSSN

SEQ ID NO: 205 - skipped

SEQ ID NO: 206 - protein, 9 aa, Synthetic construct
  source 1..9: note = OZ-A1 LCR3
QQYNNWPLT

SEQ ID NO: 207 - protein, 6 aa, Synthetic construct
  source 1..6: note = OZ-A10 LCR1
QGISSY

SEQ ID NO: 208 - skipped

SEQ ID NO: 209 - protein, 9 aa, Synthetic construct
  source 1..9: note = OZ-A10 LCR3
QQLNSYPPT

SEQ ID NO: 210 - protein, 6 aa, Synthetic construct
  source 1..6: note = OZ-A3 LCR1
QGIRND

SEQ ID NO: 211 - skipped

SEQ ID NO: 212 - protein, 9 aa, Synthetic construct
  source 1..9: note = OZ-A3 LCR3
LQHNSYPYT

SEQ ID NO: 213 - protein, 6 aa, synthetic construct
  source 1..6: note = OZ_A2_LCR1
QRASSN

SEQ ID NO: 214 - skipped

SEQ ID NO: 215 - protein, 11 aa, Synthetic construct
  source 1..11: note = OZ-A2 LCR3
QQYNTGPLMYT

SEQ ID NO: 216 - protein, 11 aa, synthetic construct
  source 1..11: note = OZ_A5_LCR1
QSLRHSNGYNY

SEQ ID NO: 217 - skipped

SEQ ID NO: 218 - protein, 8 aa, Synthetic construct
  source 1..8: note = OZ-A5 LCR3
TQALQTLT

SEQ ID NO: 219 - protein, 6 aa, Synthetic construct
  source 1..6: note = OZ-A6 LCR1
RGIRND

SEQ ID NO: 220 - skipped

SEQ ID NO: 221 - protein, 9 aa, Synthetic construct
  source 1..9: note = OZ-A6 LCR3
LQHNSYPRT

SEQ ID NO: 222 - protein, 6 aa, Synthetic construct
  source 1..6: note = OZ-A9B LCR1
QTIGSS

SEQ ID NO: 223 - skipped

SEQ ID NO: 224 - protein, 9 aa, Synthetic construct
  source 1..9: note = OZ-A9B LCR3
HQSSSLPHT

SEQ ID NO: 225 - protein, 11 aa, Synthetic construct
  source 1..11: note = OZ-A9C LCR1
QSLVYSDGNTY

SEQ ID NO: 226 - skipped

SEQ ID NO: 227 - protein, 9 aa, Synthetic construct
  source 1..9: note = OZ-A9C LCR3
MQGTHWPRT

SEQ ID NO: 228 - protein, 9 aa, Synthetic construct
  source 1..9: note = OZ-B4 LCR1
SSDVGGYNY

SEQ ID NO: 229 - skipped

SEQ ID NO: 230 - protein, 10 aa, Synthetic construct
  source 1..10: note = OZ-B4 LCR3
SSYTSSSTWV

SEQ ID NO: 231 - protein, 6 aa, Synthetic construct
  source 1..6: note = OZ-B10 LCR1
ALPKKY

SEQ ID NO: 232 - skipped

SEQ ID NO: 233 - protein, 11 aa, Synthetic construct
  source 1..11: note = OZ-B10 LCR3
YSTDSSGNHRV

SEQ ID NO: 234 - protein, 6 aa, Synthetic construct
  source 1..6: note = OZ-B11 LCR1
QSVSSN

SEQ ID NO: 235 - skipped

SEQ ID NO: 236 - protein, 9 aa, Synthetic construct
  source 1..9: note = OZ-B11 LCR3
QQYNNWPFT

SEQ ID NO: 237 - protein, 6 aa, Synthetic construct
  source 1..6: note = OZ-C4 LCR1
ALPKKY

SEQ ID NO: 238 - skipped

SEQ ID NO: 239 - protein, 11 aa, synthetic construct
  source 1..11: note = OZ_C4_LCR3
YSTDSSGNHSV

SEQ ID NO: 240 - protein, 6 aa, Synthetic construct
  source 1..6: note = OZ-C7 LCR1
SLRSYY

SEQ ID NO: 241 - skipped

SEQ ID NO: 242 - protein, 11 aa, Synthetic construct
  source 1..11: note = OZ-C7 LCR3
NSRDSSGNHWV

SEQ ID NO: 243 - protein, 6 aa, Synthetic construct
  source 1..6: note = OZ-C8 LCR1
QSISSW

SEQ ID NO: 244 - skipped

SEQ ID NO: 245 - protein, 7 aa, Synthetic construct
  source 1..7: note = OZ-C8 LCR3
QQYNSFT

SEQ ID NO: 246 - protein, 11 aa, Synthetic construct
  source 1..11: note = OZ-D2 LCR1
QSLVYSDGNTY

SEQ ID NO: 247 - skipped

SEQ ID NO: 248 - protein, 9 aa, Synthetic construct
  source 1..9: note = OZ-D2 LCR3
MQGTHWPWT

SEQ ID NO: 249 - protein, 9 aa, Synthetic construct
  source 1..9: note = OZ-D4 LCR1
SSDVGSYNL

SEQ ID NO: 250 - skipped

SEQ ID NO: 251 - protein, 10 aa, Synthetic construct
  source 1..10: note = OZ-D4 LCR3
SSYAGGNTLV

SEQ ID NO: 252 - protein, 6 aa, Synthetic construct
  source 1..6: note = OZ-D6 LCR1
RGIRND

SEQ ID NO: 253 - skipped

SEQ ID NO: 254 - protein, 9 aa, Synthetic construct
  source 1..9: note = OZ-D6 LCR3
LQHNSYPRT

SEQ ID NO: 255 - protein, 6 aa, Synthetic construct
  source 1..6: note = OZ-D9 LCR1
KLGDKY

SEQ ID NO: 256 - skipped

SEQ ID NO: 257 - protein, 9 aa, Synthetic construct
  source 1..9: note = OZ-D9 LCR3
QAWDSSTVV

SEQ ID NO: 258 - protein, 6 aa, Synthetic construct
  source 1..6: note = OZ-E3 LCR1
KLGDKY

SEQ ID NO: 259 - skipped

SEQ ID NO: 260 - protein, 9 aa, Synthetic construct
  source 1..9: note = OZ-E3 LCR3
QAWDSSTVV

SEQ ID NO: 261 - protein, 6 aa, Synthetic construct
  source 1..6: note = OZ-F2 LCR1
QSVSSN

SEQ ID NO: 262 - skipped

SEQ ID NO: 263 - protein, 8 aa, Synthetic construct
  source 1..8: note = OZ-F2 LCR3
QQYNNWRT

SEQ ID NO: 264 - protein, 9 aa, Synthetic construct
  source 1..9: note = OZ-F3 LCR1
SSDVGGYNY

SEQ ID NO: 265 - skipped

SEQ ID NO: 266 - protein, 10 aa, Synthetic construct
  source 1..10: note = OZ-F3 LCR3
SSYAGSNIWV

SEQ ID NO: 267-268 - skipped (2 entries)

SEQ ID NO: 269 - protein, 9 aa, Synthetic construct
  source 1..9: note = OZ-H2 LCR3
QQYYSTPPT

SEQ ID NO: 270 - protein, 7 aa, Synthetic construct
  source 1..7: note = OZ-H6 LCR1
QSVSSSY

SEQ ID NO: 271 - skipped

SEQ ID NO: 272 - protein, 9 aa, Synthetic construct
  source 1..9: note = OZ-H6 LCR3
QQYGSSPRT

SEQ ID NO: 273 - protein, 9 aa, Synthetic construct
  source 1..9: note = OZ-H8 LCR1
SSDVGGYNY

SEQ ID NO: 274 - skipped

SEQ ID NO: 275 - protein, 10 aa, Synthetic construct
  source 1..10: note = OZ-H8 LCR3
SSYAGSNNLV

SEQ ID NO: 276 - protein, 6 aa, Synthetic construct
  source 1..6: note = OZ-H10 LCR1
SLRSYY

SEQ ID NO: 277 - skipped

SEQ ID NO: 278 - protein, 11 aa, Synthetic construct
  source 1..11: note = OZ-H10 LCR3
NSRDSSGNHLV

SEQ ID NO: 279 - protein, 6 aa, Synthetic construct
  source 1..6: note = OZ-A4 LCR1
QSVSSY

SEQ ID NO: 280 - skipped

SEQ ID NO: 281 - protein, 10 aa, Synthetic construct
  source 1..10: note = OZ-A4 LCR3
QQRSNWPPYT

SEQ ID NO: 282 - protein, 6 aa, Synthetic construct
  source 1..6: note = OZ-B9 LCR1
QGISSF

SEQ ID NO: 283 - skipped

SEQ ID NO: 284 - protein, 9 aa, Synthetic construct
  source 1..9: note = OZ-B9 LCR3
QQLNSYPYT

SEQ ID NO: 285 - protein, 6 aa, Synthetic construct
  source 1..6: note = OZ-E11K LCR1
QSISSW

SEQ ID NO: 286 - skipped

SEQ ID NO: 287 - protein, 10 aa, Synthetic construct
  source 1..10: note = OZ-E11K LCR3
QQYNSYSPYT

SEQ ID NO: 288 - protein, 6 aa, Synthetic construct
  source 1..6: note = OZ-E11L LCR1
NIGSTS

SEQ ID NO: 289 - skipped

SEQ ID NO: 290 - protein, 11 aa, Synthetic construct
  source 1..11: note = OZ-E11L LCR3
QVWDSSSDHVV

SEQ ID NO: 291 - DNA, 24 bp, Synthetic construct
  source 1..24: mol_type = other DNA; note = OZ-A1 HCDR1 NT
ggtgggtctttcagtggttactac

SEQ ID NO: 292 - DNA, 21 bp, Synthetic construct
  source 1..21: mol_type = other DNA; note = OZ-A1 HCDR2 NT
atcaatcatagtggattcacc

SEQ ID NO: 293 - DNA, 66 bp, Synthetic construct
  source 1..66: mol_type = other DNA; note = OZ-A1 HCDR3 NT
gcgagaggcgggtattactatggttcggggagttccctctactactactactccggtatggacgtc

SEQ ID NO: 294 - DNA, 30 bp, Synthetic construct
  source 1..30: mol_type = other DNA; note = OZ-A10 HCDR1 NT
ggtggctccgtcagcagtggtagttactac

SEQ ID NO: 295 - DNA, 21 bp, Synthetic construct
  source 1..21: mol_type = other DNA; note = OZ-A10 HCDR2 NT
atctattacagtgggagcacc

SEQ ID NO: 296 - DNA, 36 bp, Synthetic construct
  source 1..36: mol_type = other DNA; note = OZ-A10 HCDR3 NT
gcgagagggtatagcagcagtcgggcttttgatatc

SEQ ID NO: 297 - DNA, 24 bp, Synthetic construct
  source 1..24: mol_type = other DNA; note = OZ-A3 HCDR1 NT
ggattcaccttcagtagctatagc

SEQ ID NO: 298 - DNA, 24 bp, Synthetic construct
  source 1..24: mol_type = other DNA; note = OZ-A3 HCDR2 NT
attagtagtagtagtagtaccata

SEQ ID NO: 299 - DNA, 39 bp, Synthetic construct
  source 1..39: mol_type = other DNA; note = OZ-A3 HCDR3 NT
gcgagagggtattactatggttcggggagtcttgactac

SEQ ID NO: 300 - DNA, 30 bp, Synthetic construct
  source 1..30: mol_type = other DNA; note = OZ-A2 HCDR1 NT
ggtggctccatcagcagtagtagttactac

SEQ ID NO: 301 - DNA, 21 bp, Synthetic construct
  source 1..21: mol_type = other DNA; note = OZ-A2 HCDR2 NT
atctattatagtgggagcacc

SEQ ID NO: 302 - DNA, 30 bp, Synthetic construct
  source 1..30: mol_type = other DNA; note = OZ-A2 HCDR3 NT
gcgagacataactactacggtatggacgtc

SEQ ID NO: 303 - DNA, 24 bp, Synthetic construct
  source 1..24: mol_type = other DNA; note = OZ-A5 HCDR1 NT
ggtgggtccttcagtggttactac

SEQ ID NO: 304 - DNA, 21 bp, Synthetic construct
  source 1..21: mol_type = other DNA; note = OZ-A5 HCDR2 NT
atcaatcatagtggaagcacc

SEQ ID NO: 305 - DNA, 54 bp, Synthetic construct
  source 1..54: mol_type = other DNA; note = OZ-A5 HCDR3 NT
gcgagggatggttcggggagttattataacgtctactactacggtatggacgtc

SEQ ID NO: 306 - DNA, 27 bp, Synthetic construct
  source 1..27: mol_type = other DNA; note = OZ-A6 HCDR1 NT
ggtggctccatcagcagtagtaactgg

SEQ ID NO: 307 - DNA, 21 bp, Synthetic construct
  source 1..21: mol_type = other DNA; note = OZ-A6 HCDR2 NT
atctatcatagtggaagcacc

SEQ ID NO: 308 - DNA, 60 bp, Synthetic construct
  source 1..60: mol_type = other DNA; note = OZ-A6 HCDR3 NT
gcgagaggccaattactatggttcggggagttggggtactactactacggtatggacgtc

SEQ ID NO: 309 - DNA, 24 bp, Synthetic construct
  source 1..24: mol_type = other DNA; note = OZ-A9B HCDR1 NT
ggtggctccatcagtagttactac

SEQ ID NO: 310 - DNA, 21 bp, synthetic construct
  source 1..21: mol_type = other DNA; note = OZ-A9B HCDR2 NT
atctattacagtgggagcacc

SEQ ID NO: 311 - DNA, 45 bp, Synthetic construct
  source 1..45: mol_type = other DNA; note = OZ-A9B HCDR3 NT
gcgagctatggttcggggagttattataactggtacttcgatctc

SEQ ID NO: 312 - DNA, 24 bp, Synthetic construct
  source 1..24: mol_type = other DNA; note = OZ-A9C HCDR1 NT
ggattcaccttcagtagctatggc

SEQ ID NO: 313 - DNA, 24 bp, Synthetic construct
  source 1..24: mol_type = other DNA; note = OZ-A9C HCDR2 NT
atatcatatgatggaagtaataag

SEQ ID NO: 314 - DNA, 45 bp, Synthetic construct
  source 1..45: mol_type = other DNA; note = OZ-A9C HCDR3 NT
gcgaaggatcaagactacggtgcctccccccacggtatggacgtc

SEQ ID NO: 315 - DNA, 24 bp, Synthetic construct
  source 1..24: mol_type = other DNA; note = OZ-B4 HCDR1 NT
ggattcaccttcagtagctatggc

SEQ ID NO: 316 - DNA, 24 bp, Synthetic construct
  source 1..24: mol_type = other DNA; note = OZ-B4 HCDR2 NT
atatcatatgatggaagtaataaa

SEQ ID NO: 317 - DNA, 36 bp, Synthetic construct
  source 1..36: mol_type = other DNA; note = OZ-B4 HCDR3 NT
gcgaaaccccgctggggttctgatgcttttgatatc

SEQ ID NO: 318 - DNA, 24 bp, Synthetic construct
  source 1..24: mol_type = other DNA; note = OZ-B10 HCDR1 NT
gggttcaccgtcagtagcaactac

SEQ ID NO: 319 - DNA, 21 bp, Synthetic construct
  source 1..21: mol_type = other DNA; note = OZ-B10 HCDR2 NT
atttatagcggtggtagcaca

SEQ ID NO: 320 - DNA, 57 bp, Synthetic construct
  source 1..57: mol_type = other DNA; note = OZ-B10 HCDR3 NT
gcgagagatattattactatggttcggggagttattataacctactactttgactac

SEQ ID NO: 321 - DNA, 30 bp, Synthetic construct
  source 1..30: mol_type = other DNA; note = OZ-B11 HCDR1 NT
ggtggctccatcagcagtggtggttactac

SEQ ID NO: 322 - DNA, 21 bp, Synthetic construct
  source 1..21: mol_type = other DNA; note = OZ-B11 HCDR2 NT
atctatcacagtgggagcacc

SEQ ID NO: 323 - DNA, 39 bp, Synthetic construct
  source 1..39: mol_type = other DNA; note = OZ-B11 HCDR3 NT
gcgagagccaattacgatattttgactgggtttgactac

SEQ ID NO: 324 - DNA, 24 bp, Synthetic construct
  source 1..24: mol_type = other DNA; note = OZ-C4 HCDR1 NT
ggattcacctttagcagctatgcc

SEQ ID NO: 325 - DNA, 24 bp, Synthetic construct
  source 1..24: mol_type = other DNA; note = OZ-C4 HCDR2 NT
attagtggtagtggtggtagcaca

SEQ ID NO: 326 - DNA, 45 bp, Synthetic construct
  source 1..45: mol_type = other DNA; note = OZ-C4 HCDR3 NT
gcgaaagatggcggggttcggggagttcctgatgcttttgatatc

SEQ ID NO: 327 - DNA, 24 bp, Synthetic construct
  source 1..24: mol_type = other DNA; note = OZ-C7 HCDR1 NT
ggattcaccttcagtagctatagc

SEQ ID NO: 328 - DNA, 24 bp, synthetic construct
  source 1..24: mol_type = other DNA; note = OZ-C7 HCDR2 NT
attagtagtagtagtagttacata

SEQ ID NO: 329 - DNA, 51 bp, Synthetic construct
  source 1..51: mol_type = other DNA; note = OZ-C7 HCDR3 NT
gcgagagatgatgggagctactcttactactactactacggtatggacgtc

SEQ ID NO: 330 - DNA, 24 bp, Synthetic construct
  source 1..24: mol_type = other DNA; note = OZ-C8 HCDR1 NT
ggatacaccttcaccggctactat

SEQ ID NO: 331 - DNA, 24 bp, Synthetic construct
  source 1..24: mol_type = other DNA; note = OZ-C8 HCDR2 NT
atcaaccctaacagtggtggcaca

SEQ ID NO: 332 - DNA, 45 bp, Synthetic construct
  source 1..45: mol_type = other DNA; note = OZ-C8 HCDR3 NT
gcgagagacttactatggttcggggagcccgatgcttttgatatc

SEQ ID NO: 333 - DNA, 30 bp, Synthetic construct; identical to SEQ ID NO: 294
  source 1..30: mol_type = other DNA; note = OZ-D2 HCDR1 NT

SEQ ID NO: 334 - DNA, 21 bp, Synthetic construct
  source 1..21: mol_type = other DNA; note = OZ-D2 HCDR2 NT
atctattacagtgggagcacc

SEQ ID NO: 335 - DNA, 42 bp, Synthetic construct
  source 1..42: mol_type = other DNA; note = OZ-D2 HCDR3 NT; note = OZ-D2 HCDR3 NT
gcgagagatcgttatagcagcagctggtacccttttgactac

SEQ ID NO: 336 - DNA, 24 bp, synthetic construct
  source 1..24: mol_type = other DNA; note = OZ-D4 HCDR1 NT
ggtggctccatcagtagttactac

SEQ ID NO: 337 - DNA, 21 bp, Synthetic construct
  source 1..21: mol_type = other DNA; note = OZ-D4 HCDR2 NT
atctattacagtgggagcacc

SEQ ID NO: 338 - DNA, 39 bp, Synthetic construct
  source 1..39: mol_type = other DNA; note = OZ-D4 HCDR3 NT
gcgaggtatagcagtggctggtccttttcttttgatatc

SEQ ID NO: 339 - DNA, 24 bp, Synthetic construct
  source 1..24: mol_type = other DNA; note = OZ-D6 HCDR1 NT
ggattcacctttgatgattatgcc

SEQ ID NO: 340 - DNA, 24 bp, Synthetic construct
  source 1..24: mol_type = other DNA; note = OZ-D6 HCDR2 NT
attagttggaatagtggtagcata

SEQ ID NO: 341 - DNA, 51 bp, synthetic construct
  source 1..51: mol_type = other DNA; note = OZ-D6 HCDR3 NT
gcaaaagatcattacgatattttgactggttattataccccctttgactac

SEQ ID NO: 342 - DNA, 24 bp, Synthetic construct
  source 1..24: mol_type = other DNA; note = OZ-D9 HCDR1 NT
ggattcacctttagtagctattgg

SEQ ID NO: 343 - DNA, 24 bp, Synthetic construct
  source 1..24: mol_type = other DNA; note = OZ-D9 HCDR2 NT
ataaagcaagatggaagtgagaaa

SEQ ID NO: 344 - DNA, 36 bp, Synthetic construct
  source 1..36: mol_type = other DNA; note = OZ-D9 HCDR3 NT
gcgagagggccaaactggggattgctttttgactac

SEQ ID NO: 345 - DNA, 30 bp, Synthetic construct; identical to SEQ ID NO: 294
  source 1..30: mol_type = other DNA; note = OZ-E3 HCDR1 NT

SEQ ID NO: 346 - DNA, 21 bp, Synthetic construct
  source 1..21: mol_type = other DNA; note = OZ-E3 HCDR2 NT
atctattacagtgggagcacc

SEQ ID NO: 347 - DNA, 42 bp, Synthetic construct
  source 1..42: mol_type = other DNA; note = OZ-E3 HCDR3 NT
gcgagaggtatagcagtggctctctactggtacttcgatctc

SEQ ID NO: 348 - DNA, 30 bp, Synthetic construct; identical to SEQ ID NO: 300
  source 1..30: mol_type = other DNA; note = OZ-F2 HCDR1 NT

SEQ ID NO: 349 - DNA, 21 bp, Synthetic construct
  source 1..21: mol_type = other DNA; note = OZ-F2 HCDR2 NT
atctattatagtgggagcacc

SEQ ID NO: 350 - DNA, 48 bp, synthetic construct
  source 1..48: mol_type = other DNA; note = OZ-F2 HCDR3 NT
gcgagaccctattactatggttcggggagtcttgatgcttttgatatc

SEQ ID NO: 351 - DNA, 24 bp, Synthetic construct
  source 1..24: mol_type = other DNA; note = OZ-F3 HCDR1 NT
ggatacagctttaccagctactgg

SEQ ID NO: 352 - DNA, 24 bp, Synthetic construct
  source 1..24: mol_type = other DNA; note = OZ-F3 HCDR2 NT
atctatcctggtgactctgatacc

SEQ ID NO: 353 - DNA, 33 bp, Synthetic construct
  source 1..33: mol_type = other DNA; note = OZ-F3 HCDR3 NT
gcgagacatcaagtgggagcctactttgactac

SEQ ID NO: 354 - DNA, 30 bp, Synthetic construct; identical to SEQ ID NO: 300
  source 1..30: mol_type = other DNA; note = OZ-H2 HCDR1 NT

SEQ ID NO: 355 - DNA, 21 bp, Synthetic construct
  source 1..21: mol_type = other DNA; note = OZ-H2 HCDR2 NT
atctattatagtgggagcacc

SEQ ID NO: 356 - DNA, 66 bp, Synthetic construct
  source 1..66: mol_type = other DNA; note = OZ-H2 HCDR3 NT
gcgagacaatggggtctaattactatggttcggggagttattaaaacggtgaactggttcgacccc

SEQ ID NO: 357 - DNA, 24 bp, Synthetic construct
  source 1..24: mol_type = other DNA; note = OZ-H6 HCDR1 NT
ggtggctccatcagtagttactac

SEQ ID NO: 358 - DNA, 21 bp, Synthetic construct
  source 1..21: mol_type = other DNA; note = OZ-H6 HCDR2 NT
atctattacagtgggagcacc

SEQ ID NO: 359 - DNA, 54 bp, Synthetic construct
  source 1..54: mol_type = other DNA; note = OZ-H6 HCDR3 NT
gcgagagaacgtattactatggttcggggattcatccctgatgcttttgatatc

SEQ ID NO: 360 - DNA, 24 bp, Synthetic construct
  source 1..24: mol_type = other DNA; note = OZ-H8 HCDR1 NT
ggatacaccttcaccggctactat

SEQ ID NO: 361 - DNA, 24 bp, Synthetic construct
  source 1..24: mol_type = other DNA; note = OZ-H8 HCDR2 NT
atcaaccctaacagtggtggcaca

SEQ ID NO: 362 - DNA, 33 bp, Synthetic construct
  source 1..33: mol_type = other DNA; note = OZ-H8 HCDR3 NT
gcgagagatatggggatcggtgcttttgatatc

SEQ ID NO: 363 - DNA, 24 bp, Synthetic construct
  source 1..24: mol_type = other DNA; note = OZ-H10 HCDR1 NT
ggattcacctttagcagctatgcc

SEQ ID NO: 364 - DNA, 24 bp, Synthetic construct
  source 1..24: mol_type = other DNA; note = OZ-H10 HCDR2 NT
attagtggtagtggtggtagcaca

SEQ ID NO: 365 - DNA, 48 bp, Synthetic construct
  source 1..48: mol_type = other DNA; note = OZ-H10 HCDR3 NT
gcgaaaggaggtactatggttcggggagttacttcgtactttgactac

SEQ ID NO: 366 - DNA, 24 bp, Synthetic construct
  source 1..24: mol_type = other DNA; note = OZ-A4 HCDR1 NT
ggattcaccttcagtagctatagc

SEQ ID NO: 367 - DNA, 24 bp, Synthetic construct
  source 1..24: mol_type = other DNA; note = OZ-A4 HCDR2 NT
attagtagtagtagtagttacata

SEQ ID NO: 368 - DNA, 39 bp, Synthetic construct
  source 1..39: mol_type = other DNA; note = OZ-A4 HCDR3 NT
gcgagttatggttcggggagttataacctctttgactac

SEQ ID NO: 369 - DNA, 24 bp, Synthetic construct
  source 1..24: mol_type = other DNA; note = OZ-B9 HCDR1 NT
ggattcacctttgataattctgcc

SEQ ID NO: 370 - DNA, 24 bp, Synthetic construct
  source 1..24: mol_type = other DNA; note = OZ-B9 HCDR2 NT
attaattgggatggtgataccaca

SEQ ID NO: 371 - DNA, 42 bp, Synthetic construct
  source 1..42: mol_type = other DNA; note = OZ-B9 HCDR3 NT
gcaaaagatattggctatggtggctacgggggctttgactac

SEQ ID NO: 372-374 - skipped (3 entries)

SEQ ID NO: 375 - DNA, 24 bp, Synthetic construct
  source 1..24: mol_type = other DNA; note = OZ-E11L HCDR1 NT
ggattcacctttagtaactattgg

SEQ ID NO: 376 - DNA, 24 bp, Synthetic construct
  source 1..24: mol_type = other DNA; note = OZ-E11L HCDR2 NT
ataaagcaagatggaagtgacaaa

SEQ ID NO: 377 - DNA, 42 bp, Synthetic construct
  source 1..42: mol_type = other DNA; note = OZ-E11L HCDR3 NT
gcgagagaatatagcagcagctggtactcgacctttgactac

SEQ ID NO: 378 - DNA, 18 bp, Synthetic construct
  source 1..18: mol_type = other DNA; note = OZ-A1 LCR1 NT
cagagtgtcagcagcaac

SEQ ID NO: 379 - skipped

SEQ ID NO: 380 - DNA, 27 bp, synthetic construct
  source 1..27: mol_type = other DNA; note = OZ-A1 LCR3 NT
cagcagtataataactggccgctcact

SEQ ID NO: 381 - DNA, 18 bp, Synthetic construct
  source 1..18: mol_type = other DNA; note = OZ-A10 LCR1 NT
cagggcattagcagttat

SEQ ID NO: 382 - skipped

SEQ ID NO: 383 - DNA, 27 bp, synthetic construct
  source 1..27: mol_type = other DNA; note = OZ-A10 LCR3 NT
caacagcttaatagttaccctccgacc

SEQ ID NO: 384 - DNA, 18 bp, Synthetic construct
  source 1..18: mol_type = other DNA; note = OZ-A3 LCR1 NT
cagggcattagaaatgat

SEQ ID NO: 385 - skipped

SEQ ID NO: 386 - DNA, 27 bp, Synthetic construct
  source 1..27: mol_type = other DNA; note = OZ-A3 LCR3 NT
ctacagcataatagttacccgtacact

SEQ ID NO: 387 - DNA, 18 bp, synthetic construct
  source 1..18: mol_type = other DNA; note = OZ-A2 LCR1 NT
cagagggctagcagcaat

SEQ ID NO: 388 - skipped

SEQ ID NO: 389 - DNA, 33 bp, Synthetic construct
  source 1..33: mol_type = other DNA; note = OZ-A2 LCR3 NT
cagcagtataatacagggccccttatgtacact

SEQ ID NO: 390 - DNA, 33 bp, Synthetic construct
  source 1..33: mol_type = other DNA; note = OZ-A5 LCR1 NT
cagagcctccggcatagtaatggatacaactat

SEQ ID NO: 391 - skipped

SEQ ID NO: 392 - DNA, 24 bp, Synthetic construct
  source 1..24: mol_type = other DNA; note = OZ-A5 LCR3 NT
acgcaagctctacaaactctcact

SEQ ID NO: 393 - DNA, 18 bp, Synthetic construct
  source 1..18: mol_type = other DNA; note = OZ-A6 LCR1 NT
cggggcattagaaatgat

SEQ ID NO: 394 - skipped

SEQ ID NO: 395 - DNA, 27 bp, Synthetic construct
  source 1..27: mol_type = other DNA; note = OZ-A6 LCR3 NT
ttacagcataatagttaccctcggacg

SEQ ID NO: 396 - DNA, 18 bp, Synthetic construct
  source 1..18: mol_type = other DNA; note = OZ-A9B LCR1 NT
cagaccattggtagtagc

SEQ ID NO: 397 - skipped

SEQ ID NO: 398 - DNA, 27 bp, Synthetic construct
  source 1..27: mol_type = other DNA; note = OZ-A9B LCR3 NT
catcagagtagtagtttacctcacact

SEQ ID NO: 399 - DNA, 33 bp, Synthetic construct
  source 1..33: mol_type = other DNA; note = OZ-A9C LCR1 NT
caaagcctcgtatacagtgatggaaacacctac

SEQ ID NO: 400 - skipped

SEQ ID NO: 401 - DNA, 27 bp, Synthetic construct
  source 1..27: mol_type = other DNA; note = OZ-A9C LCR3 NT
atgcaaggtacacactggcctcggacg

SEQ ID NO: 402 - DNA, 27 bp, Synthetic construct
  source 1..27: mol_type = other DNA; note = OZ-B4 LCR1 NT
agcagtgacgttggtggttataactat

SEQ ID NO: 403 - skipped

SEQ ID NO: 404 - DNA, 30 bp, Synthetic construct
  source 1..30: mol_type = other DNA; note = OZ-B4 LCR3 NT
agctcatatacaagcagcagcacttgggtg

SEQ ID NO: 405 - DNA, 18 bp, Synthetic construct
  source 1..18: mol_type = other DNA; note = OZ-B10 LCR1 NT
gcattgccaaaaaaatat

SEQ ID NO: 406 - skipped

SEQ ID NO: 407 - DNA, 33 bp, Synthetic construct
  source 1..33: mol_type = other DNA; note = OZ-B10 LCR3 NT
tactcaacagacagcagtggtaatcatagggta

SEQ ID NO: 408 - DNA, 15 bp, Synthetic construct
  source 1..15: mol_type = other DNA; note = OZ-B11 LCR1 NT
cagacaagcagcatg

SEQ ID NO: 409 - skipped

SEQ ID NO: 410 - DNA, 27 bp, Synthetic construct
  source 1..27: mol_type = other DNA; note = OZ-B11 LCR3 NT
cagccgactagtaaatgtccgttcact

SEQ ID NO: 411 - DNA, 18 bp, Synthetic construct
  source 1..18: mol_type = other DNA; note = OZ-C4 LCR1 NT
gcattgccaaaaaaatat

SEQ ID NO: 412 - skipped

SEQ ID NO: 413 - DNA, 33 bp, synthetic construct
  source 1..33: mol_type = other DNA; note = OZ-C4 LCR3 NT
tactcaacagacagcagtggtaatcatagtgta

SEQ ID NO: 414 - DNA, 18 bp, Synthetic construct
  source 1..18: mol_type = other DNA; note = OZ-C7 LCR1 NT
agcctcagaagctattat

SEQ ID NO: 415 - skipped

SEQ ID NO: 416 - DNA, 33 bp, Synthetic construct
  source 1..33: mol_type = other DNA; note = OZ-C7 LCR3 NT
aactcccgggacagcagtggtaaccattgggtg

SEQ ID NO: 417 - DNA, 18 bp, Synthetic construct
  source 1..18: mol_type = other DNA; note = OZ-C8 LCR1 NT
cagagtattagtagctgg

SEQ ID NO: 418 - skipped

SEQ ID NO: 419 - DNA, 21 bp, Synthetic construct
  source 1..21: mol_type = other DNA; note = OZ-C8 LCR3 NT
caacagtataatagtttcact

SEQ ID NO: 420 - DNA, 33 bp, Synthetic construct; identical to SEQ ID NO: 399
  source 1..33: mol_type = other DNA; note = OZ-D2 LCR1 NT

SEQ ID NO: 421 - skipped

SEQ ID NO: 422 - DNA, 27 bp, Synthetic construct
  source 1..27: mol_type = other DNA; note = OZ-D2 LCR3 NT
atgcaaggtacacactggccgtggacg

SEQ ID NO: 423 - DNA, 27 bp, Synthetic construct
  source 1..27: mol_type = other DNA; note = OZ-D4 LCR1 NT
agcagtgatgttgggagttataacctt

SEQ ID NO: 424 - skipped

SEQ ID NO: 425 - DNA, 30 bp, Synthetic construct
  source 1..30: mol_type = other DNA; note = OZ-D4 LCR3 NT
tcctcatatgcaggtggtaacactttggtg

SEQ ID NO: 426 - DNA, 18 bp, Synthetic construct
  source 1..18: dev_stage = OZ-D6 LCR1 NT; mol_type = other DNA
cggggcattagaaatgat

SEQ ID NO: 427 - skipped

SEQ ID NO: 428 - DNA, 27 bp, Synthetic construct
  source 1..27: mol_type = other DNA; note = OZ-D6 LCR3 NT
ttacagcataatagttaccctcggacg

SEQ ID NO: 429 - DNA, 18 bp, Synthetic construct
  source 1..18: mol_type = other DNA; note = OZ-D9 LCR1 NT
aaattgggggataaatat

SEQ ID NO: 430 - skipped

SEQ ID NO: 431 - DNA, 27 bp, Synthetic construct
  source 1..27: mol_type = other DNA; note = OZ-D9 LCR3 NT
caggcgtgggacagcagcactgtggta

SEQ ID NO: 432 - DNA, 18 bp, Synthetic construct
  source 1..18: mol_type = other DNA; note = OZ-E3 LCR1 NT
aaattgggggataaatat

SEQ ID NO: 433 - skipped

SEQ ID NO: 434 - DNA, 27 bp, Synthetic construct
  source 1..27: mol_type = other DNA; note = OZ-E3 LCR3 NT
caggcgtgggacagcagcactgtggta

SEQ ID NO: 435 - DNA, 18 bp, Synthetic construct
  source 1..18: mol_type = other DNA; note = OZ-F2 LCR1 NT
cagagtgttagcagcaac

SEQ ID NO: 436 - skipped

SEQ ID NO: 437 - DNA, 24 bp, Synthetic construct
  source 1..24: mol_type = other DNA; note = OZ-F2 LCR3 NT
cagcagtataataactggcggacg

SEQ ID NO: 438 - DNA, 27 bp, Synthetic construct
  source 1..27: dev_stage = OZ-F3 LCR1 NT; mol_type = other DNA
agcagtgacgttggtggttataactat

SEQ ID NO: 439 - skipped

SEQ ID NO: 440 - DNA, 30 bp, Synthetic construct
  source 1..30: mol_type = other DNA; note = OZ-F3 LCR3 NT
agctcatatgcaggcagcaacatttgggtg

SEQ ID NO: 441-442 - skipped (2 entries)

SEQ ID NO: 443 - DNA, 27 bp, Synthetic construct
  source 1..27: mol_type = other DNA; note = OZ-H2 LCR3 NT
cagcaatattatagtactcctccgacg

SEQ ID NO: 444 - DNA, 21 bp, synthetic construct
  source 1..21: mol_type = other DNA; note = OZ-H6 LCR1 NT
cagagtgttagcagcagctac

SEQ ID NO: 445 - skipped

SEQ ID NO: 446 - DNA, 27 bp, Synthetic construct
  source 1..27: mol_type = other DNA; note = OZ-H6 LCR3 NT
cagcagtatggtagctcacctcggacg

SEQ ID NO: 447 - DNA, 27 bp, Synthetic construct
  source 1..27: mol_type = other DNA; note = OZ-H8 LCR1 NT
agcagtgacgttggtggttataactat

SEQ ID NO: 448 - skipped

SEQ ID NO: 449 - DNA, 30 bp, Synthetic construct
  source 1..30: mol_type = other DNA; note = OZ-H8 LCR3 NT
agctcatatgcaggcagcaacaatttggta

SEQ ID NO: 450 - DNA, 18 bp, Synthetic construct
  source 1..18: mol_type = other DNA; note = OZ-H10 LCR1 NT
agcctcagaagctattat

SEQ ID NO: 451 - skipped

SEQ ID NO: 452 - DNA, 33 bp, Synthetic construct
  source 1..33: mol_type = other DNA; note = OZ-H10 LCR3 NT
aactcccgggacagcagtggtaaccatctggtg

SEQ ID NO: 453 - DNA, 18 bp, Synthetic construct
  source 1..18: mol_type = other DNA; note = OZ-A4 LCR1 NT
cagagtgttagcagctac

SEQ ID NO: 454 - skipped

SEQ ID NO: 455 - DNA, 30 bp, Synthetic construct
  source 1..30: dev_stage = OZ-A4 LCR3 NT; mol_type = other DNA
cagcagcgtagcaactggcctccgtacact

SEQ ID NO: 456 - DNA, 18 bp, Synthetic construct
  source 1..18: mol_type = other DNA; note = OZ-B9 LCR1 NT
cagggccatagcagtttt

SEQ ID NO: 457 - skipped

SEQ ID NO: 458 - DNA, 27 bp, Synthetic construct
  source 1..27: mol_type = other DNA; note = OZ-B9 LCR3 NT
caacagcgtaatagttacccgtacact

SEQ ID NO: 459 - DNA, 18 bp, Synthetic construct
  source 1..18: mol_type = other DNA; note = OZ-E11K LCR1 NT
cagagtattagtagctgg

SEQ ID NO: 460 - skipped

SEQ ID NO: 461 - DNA, 30 bp, Synthetic construct
  source 1..30: mol_type = other DNA; note = OZ-E11K LCR3 NT
caacagtataatagttattctccgtacact

SEQ ID NO: 462 - DNA, 18 bp, synthetic construct
  source 1..18: mol_type = other DNA; note = OZ-E11L LCR1 NT
aacattggaagtacaagt

SEQ ID NO: 463 - skipped

SEQ ID NO: 464 - DNA, 33 bp, Synthetic construct
  source 1..33: mol_type = other DNA; note = OZ-E11L LCR3 NT
caggtgtgggatagtagtagtgatcatgtggta

(19) United States
(12) Patent Application Publication
(10) Pub. No.: US 2026/0356922 A1
(43) Pub. Date: Nov. 12, 2026

(54) ANTIBODIES FOR ZIKA VIRUS

(71) Applicant: OMNIAB, INC., Emeryville, CA (US)

(72) Inventors: Yuxing Li, Emeryville, CA (US); Xiaoran Shang, Emeryville, CA (US)

(21) Appl. No.: 19/147,638

(22) PCT Filed: Dec. 12, 2023
(86) PCT No.: PCT/US2023/083586

§ 371 Date: Jul. 11, 2025

Related U.S. Application Data
(60) Provisional application No. 63/480,899, filed on Jan. 20, 2023.

(51) Int. Cl. C07K 16/116; A61P 31/14

(52) U.S. Cl. CPC C07K 16/116; A61P 31/14

(57) ABSTRACT

The present disclosure provides antibodies and fragments thereof that specifically bind to Zika virus E protein and in some cases bind to and/or neutralize Zika virus. Compositions including one or more antibodies, fragments thereof, polypeptides that bind and/or neutralize Zika virus, or nucleic acids encoding such molecules are described herein.

### INCORPORATION BY REFERENCE TO ANY PRIORITY APPLICATIONS

This application claims the benefit of U.S. Provisional Ser. No. 63/480,899, filed Jan. 20, 2023; which is hereby incorporated by reference in its entirety.

### SEQUENCE LISTING

The present application is being filed along with a Sequence Listing in electronic format. The Sequence Listing is provided as a file entitled OMAB3009WOseqlist.xml, created Dec. 5, 2023, which is 456,582 bytes in size. The information in the electronic format of the Sequence Listing is incorporated herein by reference in its entirety.

### BACKGROUND OF THE INVENTION

### Field of the Invention

The application relates to antibodies and antibody fragments with Zika virus binding and neutralizing activity, compositions comprising such antibodies, and methods of characterizing such antibodies.

### Description of the Related Art

Zika virus (ZIKV) is a member of flavivirus family that emerged as an infectious agent causing global health crisis during recent epidemics. ZIKV infection can cause Guillain-Barré syndrome in adults, and severe fetal neuronal formations and fetal death during pregnancy. ZIKV infection is primarily transmitted by mosquito bite, while sexual transmission and vertical transmission from infected pregnant women to fetus also contribute to the recent epidemic. Ideally, an effective ZIKV vaccine should provide sterilizing immunity that blocks the initial viral dissemination to prevent subsequent infection-caused morbidity. Currently, there is no approved ZIKV vaccine for disease prevention.

ZIKV has an ~11 kb RNA genome and the viral mRNA translates into a polyprotein that is cleaved into 3 structural proteins-capsid (C), pre-membrane/membrane (prM/M), and envelope (E), and 7 non-structural proteins (NS). Among these structural: proteins, E protein is the focus of neutralizing antibody (nAb) and vaccine development. The Zika virus E protein composing three ectodomains (DI, DII, and DIII) displays homo-dimeric conformation on mature virion. There are three major nAb epitopes on E protein targeted by nAbs identified: E-dimer epitope (EDE), a quaternary epitope formed across the interface of two E protomers arranged in a head-to-tail conformation (1, 2), domain III lateral ridge epitope, DIII LR (3, 4), and a quaternary epitope located on DII at the dimer-dimer interface represented by monoclonal antibody (mAb) ZIKV-117 (5, 6). An immunodominant and suboptimal epitope, namely fusion loop epitope (FLE), consisting of 11 amino acid residues within DII and sharing high degree of sequence homology with other flavivirus E proteins, elicits highly abundant antibodies that show broad flavivirus cross-reactivity but low or absent neutralization capacity.

The M and E proteins expressed as the prM-E form is a common antigen choice for current vaccine candidates against ZIKV, as nAbs against prM-E can prevent viral entry. The platforms of current vaccine candidates include 1) inactivated (7, 8) or live attenuated virus (9), or 2) DNA- (10-12) or RNA-platforms (13, 14), with selected vaccine candidates shown to be safe and immunogenic in healthy adults from phase I trials in 2017 (8, 11). However, due to the antigenic nature of the nascent form of ZIKV prM-E, these vaccine candidates will likely elicit a mixture of diverse antibody responses including: (i) protective antibodies targeting nAb epitopes such as the EDE and DIII LR, and (ii) weak or non-neutralizing antibodies that cross-react with DENV (Dengue virus) E proteins and potentially cause antibody-dependent enhancement (ADE) of DENV infection (15, 16) in individuals immunized with current ZIKV vaccine candidates, given that a majority of regions experiencing ZIKV epidemics are mostly endemic for DENV.

### SUMMARY OF THE INVENTION

The present disclosure provides antibodies and fragments thereof that specifically bind to and in some cases inhibit Zika virus (ZIKV).

In some embodiments, an antibody or antibody fragment is provided that binds to the E protein of Zika virus, wherein the antibody or antibody fragment comprise: (a) a heavy chain variable domain comprising heavy chain CDR1, CDR2 and CDR3 regions that are identical to heavy chain CDR1, CDR2 and CDR3 regions indicated in the heavy chain sequences selected from FIG. 6, wherein the CDR1, CDR2 and CDR3 regions are from the same antibody heavy chain; or (b) a variant of the heavy chain variable domain of (a) that is otherwise identical to said variable domain except for up to five amino acid substitutions in the collective CDRH1, CDRH2 and CDRH3 regions of the variable domain of (a).

In some embodiments, the antibody or antibody fragment further comprise: (c) a light chain variable domain comprising light chain CDR1, CDR2 and CDR3 regions that are identical to light chain CDR1, CDR2 and CDR3 regions indicated in the light chain sequences selected from FIG. 7, wherein the CDR1, CDR2 and CDR3 regions are from the same antibody light chain; or (d) a variant of the light chain variable domain of (c) that is otherwise identical to said variable domain except for up to five amino acid substitutions in the collective CDRH1, CDRH2 and CDRH3 regions of the variable domain of (c).

In some embodiments, the antibody or antibody fragment comprise: a heavy chain variable domain comprising an amino acid sequence that is at least 90% (e.g., at least 91%, 92%, 93%, 94%, 95%, 96%, 97%, 98%, 99%, or 100%) identical to the amino acid sequence of the heavy chain variable domain of an antibody selected from FIG. 6.

In some embodiments, the antibody or antibody fragment further comprise a light chain variable domain comprising an amino acid sequence that is at least 90% (e.g., at least 91%, 92%, 93%, 94%, 95%, 96%, 97%, 98%, 99%, or 100%) identical to the light chain variable domain of the same antibody, selected from FIG. 7.

In some embodiments, the heavy chain variable domain and the light chain variable domain of the antibody or antibody fragment are present in separate polypeptides.

In some embodiments, the heavy chain variable domain and the light chain variable domain of the antibody or antibody fragment are present in a single polypeptide.

In some embodiments, the antibody or antibody fragment are covalently linked to a peptide or non-peptide moiety.

In some embodiments, the non-peptide moiety is a synthetic polymer, a poly(ethylene glycol) polymer, a covalently linked lipid moiety, a fatty acid moiety a polysaccharide moiety or a carbohydrate moiety.

In some embodiments, the peptide moiety is an antibody light chain, an antibody heavy chain, or an antigen-binding fragment of one of the foregoing.

In some embodiments, the antibody is a single chain Fv (scFv) antibody, a heavy chain-only antibody, a VHH, knob or picobody.

In some embodiments, the antibody or antibody binding fragment neutralizes Zika virus.

In some embodiments, a composition comprising the antibody or antibody fragment of any of the embodiments disclosed herein is provided.

In some embodiments, a nucleic acid encoding an antibody or antibody fragment of any one of the embodiments disclosed herein, or the complement of said nucleic acid, is provided.

In some embodiments, a composition comprising the nucleic acid of any of the embodiments disclosed herein is provided.

In some embodiments, a pharmaceutical composition is provided comprising: a) the antibody or antibody fragment of any one of the antibody or antibody fragment embodiments disclosed herein; and b) a pharmaceutically acceptable carrier.

In some embodiments, a pharmaceutical composition is provided comprising: a) the nucleic acid of any of the nucleic acid embodiments disclosed herein; and b) a pharmaceutically acceptable carrier.

In some embodiments, a pharmaceutical composition comprising any of the antibody or antibody fragment embodiments disclosed herein is provided. In some embodiments, the antibody is encapsulated in a liposome.

In some embodiments, a pharmaceutical composition comprising any of the nucleic acid embodiments disclosed herein is provided and in some embodiments, the nucleic acid may be encapsulated in a liposome.

### BRIEF DESCRIPTION OF THE DRAWINGS

FIG. 1A-F depict an assay where sRecE-CC_FLE is able to stimulate nAb responses in transgenic animals that express naïve human Ig loci with substantial portion focused on ZIKV E dimer-specific epitopes. FIG. 1A depicts an immunization schedule. Two female transgenic mice, OM5.1 and OM5.2, at age of 20-21 weeks old, were immunized with sRecE-CC_FLE via subcutaneous (s.c.) route or intraperitoneal (IP) route at indicated time points. FIG. 1B depicts immune sera neutralization specificity mapping by neutralization depletion with various sRecE-based antigens (sRecE Ags). Terminal sera were pre-mixed with sRecE variant antigens to deplete cognate serum neutralizing antibodies prior to ZIKV H/PF/2013 RVP incubation, with mAbs of ZV67 (DIII specific) and EDE1-C8 (dimer context specific) as controls. Mock, medium containing no antigen was added to immune sera or mAb for comparison. FIG. 1C depicts CC_FLE reactive memory B cell sorting using splenocytes from animal OM5.1. FIG. 1D depicts ELISA binding profiles of selected CC_FLE-elicited mAbs. Reference mAbs include EDE1-C8 (EDE-specific) and Z004 (DIII-specific) FIG. 1E depicts epitope clustering by BLI-based Ab competition assay. Left, assay scheme; Right, relative CC_FLE binding signal after co-incubation of test mAb and competitor mAbs. FIG. 1F depicts ZIKV H/PF/2013 RVP neutralization.

FIG. 2A-C depict immunogenicity of sRecE-CC_FLE in transgenic animals that express naïve human Ig loci. FIG. 2A depicts an immunization schedule. Two female transgenic mice, OM5.1 and OM5.2, at age of 20-21 weeks old, were immunized with 20 μg of sRecE-CC_FLE formulated in TiterMax Gold adjuvant via subcutaneous (s.c.) route on weeks 0, and 4. OM5.1 received another immunization on week 36 with 20 μg of sRecE-CC FLE formulated in Sigma Adjuvant System (SAS) via intraperitoneal (IP) route on week 36 and terminated on week 38. Another mouse, OM5.2 was terminated on week 6, and received additional immunization with 20 μg of sRecE-CC_FLE without any adjuvant via IP route, four days before week 6. FIG. 2B depicts an assay where transgenic mouse sera (weeks 5 and 38) was assessed for neutralization activity against ZIKV H/PF/2013 RVP, with pre-immune serum as negative control. FIG. 2C depicts immune sera neutralization activity depletion by various sRecE-based antigens (sRecE Ags). Terminal sera were pre-mixed with sRecE variant antigens to deplete cognate serum neutralizing antibodies prior to ZIKV H/PF/2013 RVP incubation, with mAbs of ZV67 (DIII specific) and EDE1-C8 (dimer specific) as controls. Mock, medium containing no antigen was added to immune sera or mAb for comparison.

FIG. 3A-B depict an experiment where dimeric sRecE-CC_FLE elicits neutralizing antibody response to epitopes distinctive from monomeric sRecE-WT. FIG. 3A depicts immune sera neutralization activity depletion by various sRecE-based antigens (sRecE Ags). Pooled day 42 sera from mice immunized with sRecE-WT or CC_FLE immunogen formulated with Adjuplex were pre-mixed with sRecE variant antigens to deplete serum neutralizing antibodies prior to ZIKV H/PF/2013 RVP incubation, with mAbs of ZV67 (DIII specific) and EDE1-C8 (dimer specific) as controls. Mock, medium containing no antigen was added to immune sera or mAb for comparison. FIG. 3B depicts summary of depletion effect of sRecE Ags on the neutralization activity of immune sera or mAbs. Neutralization Depletion Effect of adding an antigen to each immune serum was calculated as 100*(1−(Maximal Neutralization Percentage_Ag/Maximal Neutralization Percentage Mock)), while the effect of an antigen to each mAb, 100*(1−(Maximal Neutralization Percentage_Ag/Maximal Neutralization Percentage Mock)).

FIG. 4A-E depicts cryoelectron microscopy (CryoEM) structure of OZ-D4 mAb in complex with ZIKV E stabilized dimer CC_Core. FIG. 4A depicts 3.45 Å resolution cryoEM map colored by chains of bound OZ-D4 fabs: heavy chain, light chain. Density corresponding ZIKV E dimer is shown in grey. FIG. 4B depicts overall structure of OZ-D4-CC_Core complex fitted into the semitransparent CryoEM density shown in grey. ZIKV E dimer is broken down by domains: domain I, domain II, domain III, fusion loop. FIG. 4C depicts overall view of OZ-D4 binding interface with both protomers of ZikV E dimer. Semi-transparent surface representation of OZ-D4 epitope on ZikV E dimer interface: protomer A binding surface and protomer B. FIG. 4D depicts a cartoon representation of interacting residues of the OZ-D4 light chain (left) and OZ-D4 heavy chain (right) with Zikv E dimer, FIG. 4E depicts footprint comparison of OZ-D4 with established EDE mAbs: EDE1-C8 (pdb 5 lbs) and EDE2-All (pdb 5lcv). Binding epitopes on the surface of the zika dimer are shown in dark grey.

FIG. 5A-E depicts CryoEM data of OZ-D4 mAb complex with ZIKV E stabilized dimer CC_Core. FIG. 5A depicts representative 2D-class averages, FIG. 5B depicts a FSC plot with global resolution reported at FSC 0.143. FIG. 5C depicts a directional distribution plot. FIG. 5D depicts a density map colored by local resolution. FIG. 5E depicts a table with data collection, refinement and validation statistics.

FIG. 6 depicts variable heavy chain sequences with the CDR1, 2, and 3 sequences appearing in order for each variable heavy chain sequence. The CDRs are indicated by bold and underlined text.

FIG. 7 depicts variable light chain sequences with the CDR1, 2, and 3 sequences appearing in order for each variable light chain sequence except SEQ ID NO; 92 which only possess a LCDR3. The CDRs are indicated by bold and underlined text.

### DEFINITIONS

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. Also encompassed by the term are Fab′, Fv, F(ab′)2, and or other antibody fragments that retain specific binding to antigen, and monoclonal antibodies. An antibody may be monovalent or multivalent, e.g., bivalent.

The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Intact antibodies typically contain two heavy chains and two light chains. “Antibody fragments” comprise a portion of an intact antibody, for example, the antigen binding or variable region of the intact antibody. Examples of antibody fragments include Fab, Fab′, F(ab′) 2, and Fv fragments; diabodies; linear antibodies (Zapata et al., Protein Eng. 8 (10): 1057-1062 (1995)); picobodies or “knobs” (MacGregor et al., WO2022/241058; MacPherson et al., PLOS Biol 18 (9): e3000821), single chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′) 2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. Even a single variable domain (or half of an Fv comprising only three CDRs specific for an antigen) can have the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (Vu) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993), all of which are expressly incorporated by reference in their entireties.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. An anti-Zika virus E protein antibody binds specifically to an epitope within a Zika virus E protein polypeptide. Non-specific binding would refer to binding with an affinity of less than about 10⁷ M, e.g., binding with an affinity of 10⁶ M, 10⁵ M, or 10⁴ M, etc.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept of Health and Human Services, “Sequences of proteins of immunological interest” (1991); by Chothia et al., J. Mol. Biol. 196:901-917 (1987); and MacCallum et al., J. Mol. Biol. 262:732-745 (1996), and Lefranc, M.-P. et al., Dev. Comp. Immunol., 2003 27:55-77, where the definitions include overlapping or subsets of amino acid residues when compared against each other. Nevertheless, application of either definition to refer to a CDR of an antibody or grafted antibodies or variants thereof is intended to be within the scope of the term as defined and used herein. As indicated below, the Chothia, Kabat and MacCallum method can be used to define the CDRs.

TABLE 1
CDR DEFINITIONS

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, or ungulates (e.g., equines, bovines, ovines, porcines, or caprines), etc.

A “therapeutically effective amount” or “efficacious amount” refers to the amount of an anti-Zika virus E protein antibody that, when administered to a mammal or other subject for treating a disease, is sufficient to affect such treatment for the disease. The “therapeutically effective amount” will vary depending on the anti-Zika virus E protein antibody, the disease and its severity and the age, or weight, etc., of the subject to be treated.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, or tissue samples.

As used herein, the term “collectively” in the context of a variant of an antibody variable domain that is otherwise identical to the antibody variable domain except for a defined number of amino acid substitutions “in the collective CDR regions of the antibody variable domain”, indicates that the number of amino acid substitutions is counted using all six CDRs. Explained by example, if the variant has 5 amino acid substitutions relative to the antibody variable domain, then the six CDRs of the variant, combined, have a total of 5 amino acid substitutions relative to the antibody variable domain. This phrase is not intended to mean that each CDR has a defined number of amino acid substitutions.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antibody” includes a plurality of such antibodies and reference to “the anti-Zika virus E protein antibody” includes reference to one or more anti-Zika virus E protein antibodies and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Immunoglobulin refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd, or 214 amino acids) generally comprise a variable domain encoded by an exon comprising one or more variable region gene(s) and one or more joining region gene(s) at the NH₂-terminus (about 110 amino acids) and constant domain encoded by a kappa or lambda constant region gene at the COOH-terminus. Full-length immunoglobulin “heavy chains” (about 50 Kd, or 446 amino acids), similarly comprise (1) a variable domain (about 116 amino acids) encoded by an exon comprising one or more variable region genes, one or more diversity region genes and one or more joining region genes, and (2) one of the aforementioned constant domains comprising one or more constant region genes, e.g., alpha, gamma, delta, epsilon or mu (encoding about 330 amino acids). The immunoglobulin heavy chain constant region genes encode for the antibody class, i.e., isotype (e.g., IgM or IgG1).

As used herein, the term “antibody” refers to a protein comprising at least one, and preferably two, heavy (H) chain variable domains (abbreviated herein as VH), and at least one and preferably two light (L) chain variable domains (abbreviated herein as VL). An ordinarily skilled artisan will recognize that the variable domain of an immunological chain is encoded in gene segments that must first undergo somatic recombination to form a complete exon encoding the variable domain. There are three types of regions or gene segments that undergo rearrangement to form the variable domain: the variable region comprising variable genes, the diversity region comprising diversity genes (in the case of an immunoglobulin heavy chain), and the joining region comprising joining genes. The VH and VL domains can be further subdivided into regions of hypervariability, termed “complementarity determining regions” (“CDRs”) interspersed with regions that are more conserved, termed “framework regions” (“FRs”). The extent of the FRs and CDRs has been precisely defined (see, Kabat et al. (1991) Sequences of Proteins of Immunological Interest, Fifth Edition, U.S. Department of Health and Human Services, NIH Publication No. 91-3242; and Chothia et al. (1987) J. Mol. Biol. 196:901-17, which are hereby incorporated by reference). Each VH and VL domain is generally composed of three CDRs and four FRs, arranged from amino-terminus to carboxy-terminus in the following order: FR1, CDR1, FR2, CDR2, FR3, CDR3, FR4. The antigen binding fragment of an antibody (or simply “antibody portion,” or “fragment”), as used herein, refers to one or more fragments of a full-length antibody that retain the ability to specifically bind to an antigen (e.g., CD3).

Examples of binding fragments encompassed within the term “antigen binding fragment” of an antibody include (i) an Fab fragment, a monovalent fragment consisting of the VL, VH, CL and CH1 domains; (ii) an F(ab′) 2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al. (1989) Nature 341:544-46), which consists of a VH domain; (vi) a single variable domain on a heavy chain (VHH) antibody (nanobody); (vii) a single domain shark-derived variable domain of new antigen receptor (VNAR); (viii) a picobody or “knob” (MacGregor et al., WO2022/241058; MacPherson et al., PLOS Biol 18 (9): e3000821), (ix) an isolated complementarity determining region (CDR); (x) a single chain Fv (scFv) (consisting of the VH and VL domains of a single arm of an antibody joined by a synthetic linker using recombinant means such that the VH and VL domains pair to form a monovalent molecule); (xi) diabodies (consisting of two scFvs in which the VH and VL domains are joined such that they do not pair to form a monovalent molecule; the VH of each one of the scFv pairs with the VL domain of the other scFv to form a bivalent molecule); (xii) multi-specific antibodies, such as bi-specific antibodies, consisting of at least two antigen binding regions, each region binding a different epitope. In some embodiments, a subject antibody fragment is a Fab fragment. In some embodiments, a subject antibody fragment is a single-chain antibody (scFv).

These antibody fragments are obtained using conventional techniques known to those skilled in the art, and the fragments are screened for utility in the same manner as are intact antibodies.

An antibody may further include a heavy and/or light chain constant domain to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected, e.g., by disulfide bonds. The heavy chain constant domain is comprised of three gene segments, CHI, CH2 and CH3. The light chain constant domain is comprised of one gene, CL. The variable domains of the heavy and/or light chains contain a binding domain that interacts with an antigen. The constant domains of the antibodies typically mediate the binding of the antibody to host tissues or factors, including various cells of the immune system (e.g., effector cells) and the first component (C1q) of the classical complement system.

By “polynucleotide encoding an artificial immunoglobulin locus” or “artificial immunoglobulin chain” is meant a recombinant polynucleotide comprising multiple immunoglobulin regions, e.g., a variable (V) region or gene segment comprising V genes, a joining (J) gene region or gene segment comprising J genes, a diversity (D) region or gene segment comprising D genes in the case of a heavy chain locus and/or at least one constant (C) region comprising at least one C gene. Preferably, each region of the variable domain, e.g., V, D, or J region, comprises or spans at least two genes of the same type. For example, a variable region as used herein comprises at least two variable genes, a joining region comprises at least two joining genes and a diversity region comprises two diversity genes. A constant region may comprise only one constant gene, e.g., a k gene or λ gene, or multiple genes, e.g., CHI, CH2, and CH3.

“Enhancer sequences” or “enhancer” as used herein refers to sequences that have been identified near many active genes by nuclease digest and hypersensitivity to degradation. Hypersensitive sites may precede promoter sequences and the strength of their activity was correlated with the DNA sequence. Linkage to reporter genes showed elevated transcription if enhancer function was present (Mundt et al., J. Immunol., 166, 3315 [2001]). In the IgH locus two important transcription or expression regulators have been identified, Eu and the 3′E at the end of the locus (Pettersson et al., Nature, 344, 165 [1990]). In the mouse the removal of the whole 3′ regulatory region (containing hs3a, hs1,2, hs3b and hs4) allows normal early B-cell development but abrogates class-switch recombination (Vincent-Fabert et al., Blood, 116, 1895 [2010]) and possibly prevents the optimization of somatic hypermutation (Pruzina et al., Protein Engineering, Design and Selection, 1, [2011]). The regulatory function to achieve optimal isotype expression is particularly desirable when transgenic human IgH genes are being used. Transgene constructs with incomplete 3′E region, usually only providing the hs1,2 element, led to disappointing expression levels in transgenic mice even when the endogenous IgH locus was knocked-out. As a consequence, only few antigen-specific fully human IgGs have been isolated from constructs produced in the last 20 years (Lonberg et al., Nature 368, 856 [1994]; Nicholson et al., J. Immunol., 163, 6898 [1999]; Davis et al., Cancer Metastasis Rev. 18, 421 [1999]; Pruzina et al., Protein Engineering, Design and Selection, 1, [2011]). In the rat IgH locus, the 3′E region has only been poorly analyzed. A comparison of mouse and rat sequences did not allow identification of hs4, the crucial 4^{th} element with additional important regulatory sequences further downstream (Chatterjee et al., J. Biol. Chem., 286, 29303 [2011]). The polynucleotides of the present invention advantageously provide optimal expression due, at least in part, to the inclusion of a rat 3′ enhancer since chimeric polynucleotides lacking this 3′ enhancer result in impaired isotype switching and low IgG expression.

“Artificial Ig locus” as used herein may refer to polynucleotides that (e.g., a sequence comprising V-, D-, and/or J regions in the case of heavy chain, or V- and/or J regions in the case of light chain, and optionally a constant region for either or both a heavy and light chain) that are unrearranged, partially rearranged, or rearranged. Artificial Ig loci include artificial Ig light chain loci and artificial Ig heavy chain loci. In one embodiment, an artificial immunoglobulin locus of the invention is functional and capable of rearrangement and producing a repertoire of immunoglobulin chains. In a preferred embodiment, the variable domain or portion thereof of a polynucleotide disclosed herein comprises genes in natural configuration, i.e., naturally occurring sequences of an human Ig gene segment, degenerate forms of naturally occurring sequences of a human Ig gene segment, as well as synthetic sequences that encode a polypeptide sequence substantially identical to the polypeptide encoded by a naturally occurring sequence of a human Ig gene segment. In another preferred embodiment, the polynucleotide comprises a variable domain or portion thereof in a natural configuration found in humans. For example, a polynucleotide encoding an artificial Ig heavy chain as disclosed herein may comprise in natural configuration at least two human V genes, at least two D genes, at least two J genes or a combination thereof.

In a preferred embodiment, an artificial Ig locus comprises a non-human C region gene and is capable of producing a repertoire of immunoglobulins including chimeric immunoglobulins having a non-human C region. In one embodiment, an artificial Ig locus comprises a human C region gene and is capable of producing a repertoire of immunoglobulins including immunoglobulins having a human C region. In one embodiment, an artificial Ig locus comprises an “artificial constant region gene”, by which is meant a constant region gene comprising nucleotide sequences derived from human and non-human constant regions genes. For example, an exemplary artificial C constant region gene is a constant region gene encoding a human IgG CHI domain and rat IgG CH2 and CH3 domain.

In some embodiments, an artificial Ig heavy chain locus lacks CHI, or an equivalent sequence that allows the resultant immunoglobulin to circumvent the typical immunoglobulin: chaperone association. Such artificial loci provide for the production of heavy chain-only antibodies in transgenic animals which lack a functional Ig light chain locus and hence do not express functional Ig light chain. Such artificial Ig heavy chain loci are used in methods contemplated herein to produce transgenic animals lacking a functional Ig light chain locus, and comprising an artificial Ig heavy chain locus, which animals are capable of producing heavy chain-only antibodies. Alternatively, an artificial Ig locus may be manipulated in situ to disrupt CHI or an equivalent region and generate an artificial Ig heavy chain locus that provides for the production of heavy chain-only antibodies. Regarding the production of heavy chain-only antibodies in light chain-deficient mice, see for example Zou et al., JEM, 204:3271-3283, 2007.

By “human idiotype” is meant a polypeptide sequence present on a human antibody encoded by an immunoglobulin V-gene segment. The term “human idiotype” as used herein includes both naturally occurring sequences of a human antibody, as well as synthetic sequences substantially identical to the polypeptide found in naturally occurring human antibodies. By “substantially” is meant that the degree of amino acid sequence identity is at least about 85%-95%. Preferably, the degree of amino acid sequence identity is greater than 90%, more preferably greater than 95%.

By a “chimeric antibody” or a “chimeric immunoglobulin” is meant an immunoglobulin molecule comprising a portion of a human immunoglobulin polypeptide sequence (or a polypeptide sequence encoded by a human Ig gene segment) and a portion of a non-human immunoglobulin polypeptide sequence. The chimeric immunoglobulin molecules of the present invention are immunoglobulins with non-human Fc-regions or artificial Fc-regions, and human idiotypes. Such immunoglobulins can be isolated from animals of the invention that have been engineered to produce chimeric immunoglobulin molecules.

By “artificial Fc-region” is meant an Fc-region encoded by an artificial constant region gene.

The term “Ig gene segment” as used herein refers to regions of DNA encoding various portions of an Ig molecule, which are present in the germline of non-human animals and humans, and which are brought together in B cells to form rearranged Ig genes. Thus, Ig gene segments as used herein include V gene segments, D gene segments, J gene segments and C gene segments.

The term “human Ig gene segment” as used herein includes both naturally occurring sequences of a human Ig gene segment, degenerate forms of naturally occurring sequences of a human Ig gene segment, as well as synthetic sequences that encode a polypeptide sequence substantially identical to the polypeptide encoded by a naturally occurring sequence of a human Ig gene segment. By “substantially” is meant that the degree of amino acid sequence identity is at least about 85%-95%. Preferably, the degree of amino acid sequence identity is greater than 90%, more preferably greater than 95%

Polynucleotides related to the present invention may comprise DNA or RNA and may be wholly or partially synthetic. Reference to a nucleotide sequence as set out herein encompasses a DNA molecule with the specified sequence, and encompasses an RNA molecule with the specified sequence in which U is substituted for T, unless context requires otherwise.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent sequence identity between two sequences may be accomplished using a mathematical algorithm. In a preferred embodiment, the percent identity between two amino acid sequences is determined using the Needleman and Wunsch ((1970) J. Mol. Biol. 48:444-53) algorithm, which has been incorporated into the GAP program in the GCG software package (available online at gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna. CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17), which has been incorporated into the ALIGN program (version 2.0), using a PAM120 weight residue table, a gap length penalty of 12 and a gap penalty of 4.

### DETAILED DESCRIPTION

Provided herein are polynucleotides and polypeptides encoding or comprising recombinant or artificial antibodies or antibody fragments, and compositions comprising such antibodies and fragments thereof, and methods for characterizing such antibodies. The polypeptides, antibodies and antibody fragments disclosed herein can bind to Zika virus E protein and certain embodiments are capable of neutralizing ZIKV infection.

For example, the antibodies and antibody fragments disclosed herein can bind to Zika virus E protein with an affinity of at least about 10⁷ M, at least about 108 M, at least about 109 M, at least about 1010 M, at least about 1011 M, or at least about 1012 M, or greater than 1012 M.

The antibodies or antibody fragments disclosed herein can in some embodiments reduce binding of Zika virus E protein to a receptor in a mammalian cell. For example, in some embodiments the antibodies or antibody fragments disclosed herein can reduce binding of Zika virus E protein to a Zika virus E protein receptor in a mammalian cell by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the degree of binding between Zika virus E protein and the Zika virus E protein receptor in a mammalian cell in the absence of the antibodies or antibody fragments disclosed herein.

In some embodiments the antibodies or antibody fragments disclosed herein can reduce ZIKV infectivity by binding the zika virus E protein. Antibodies that bind to a virus and interfere with its ability to infect a cell are known as “neutralizing antibodies.” For example, in some embodiments the antibodies or antibody fragments disclosed herein can reduce ZIKV infectivity by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to ZIKV infectivity in the absence of the antibodies or antibody fragments disclosed herein.

The term “antibody” refers to a protein comprising one or more (e.g., one or two) heavy chain variable regions (VH) and/or one or more (e.g., one or two) light chain variable regions (VL), or subfragments thereof capable of binding an epitope. The VH and VL regions can be further subdivided into regions of hypervariability, termed “complementarity determining regions (CDR)”, interspersed with regions that are more conserved, termed “framework regions (FR)”. The extent of the FR and CDRs has been precisely defined (see, Kabat, et al. (1991) Sequences of Proteins of Immunological Interest, Fifth Edition, U.S. Department of Health and Human Services, NIH Publication No. 91-3242; Chothia et al. (1987) J. Mol. Biol. 196:901-917). A VH can comprise three CDRs and four FRs arranged from N-terminus to C-terminus in the following order: FR1, CDR1, FR2, CDR2, FR3, CDR3, FR4. Similarly, a VL can comprise three CDRs and four FRs arranged from N-terminus to C-terminus in the following order: FR1, CDR1, FR2, CDR2, FR3, CDR3, FR4.

The VH or VL chain of an antibody can further include a or part of a heavy or light chain constant region, to thereby form a heavy or light immunoglobulin chain, respectively. In one embodiment, the antibody is a tetramer of two heavy and two light chains, wherein the heavy and light chains are interconnected by, for example, disulfide bonds. The heavy chain constant region is comprised of three domains, CH1, CH2 and CH3. The light chain constant region is comprised of one domain, CL. The variable regions of the heavy and light chains comprise binding regions that interact with antigen. The constant regions of the antibodies typically mediate the binding of the antibody to host tissues and factors, including various cells of the immune system and the first component of the complement system. The term “antibody” includes intact immunoglobulins of types IgA, IgG, IgE, IgD, IgM and subtypes thereof. In some embodiments, a subject antibody is an IgG isotype. As used herein the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g. gamma (encoding about 330 amino acids). In some embodiments, a subject antibody comprises full-length immunoglobulin heavy chain and a full-length immunoglobulin light chain.

In some embodiments, a subject antibody does not comprise a full-length immunoglobulin heavy chain and a full-length immunoglobulin light chain, and instead comprises antigen-binding fragments of a full-length immunoglobulin heavy chain and/or a full-length immunoglobulin light chain. In some embodiments, the antigen-binding fragments are contained on separate polypeptide chains; in other embodiments, the antigen-binding fragments are contained within a single polypeptide chain. The term “antigen-binding fragment” refers to one or more fragments of a full-length antibody that are capable of specifically binding to Zika virus E protein as described above. Examples of binding fragments include (i) a Fab fragment (a monovalent fragment consisting of the VL, VH, CL and CH1 domains; (ii) a F(ab′)₂ fragment (a bivalent fragment comprising two Fab fragments linked by a disulfide bridge at the hinge region; (iii) an Fd fragment (consisting of the VH and CH1 domains); (iv) an Fv fragment (consisting of the VH and VL domains of a single arm of an antibody); (v) a dAb fragment (consisting of the VH domain); (vi) an isolated CDR; (vii) a single chain Fv (scFv) (consisting of the VH and VL domains of a single arm of an antibody joined by a synthetic linker using recombinant means such that the VH and VL domains pair to form a monovalent molecule); (viii) diabodies (consisting of two scFvs in which the VH and VL domains are joined such that they do not pair to form a monovalent molecule; the VH of each one of the scFv pairs with the VL domain of the other scFv to form a bivalent molecule); (ix) multi-specific antibodies (e.g., bi-specific or tri-specific antibodies), consisting of at least two antigen binding regions, each region binding a different epitope). In some embodiments, a subject antibody fragment is a Fab fragment. In some embodiments, a subject antibody fragment is a single-chain antibody (scFv).

In some embodiments, a subject antibody is a recombinant or modified antibody, e.g., a chimeric, humanized, deimmunized or an in vitro generated antibody. The term “recombinant” or “modified” antibody as used herein is intended to include all antibodies that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

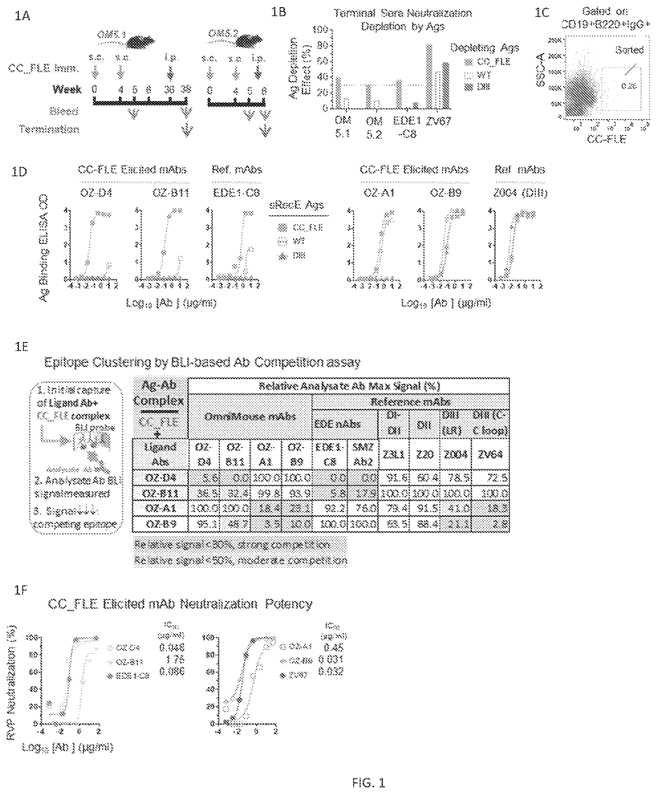

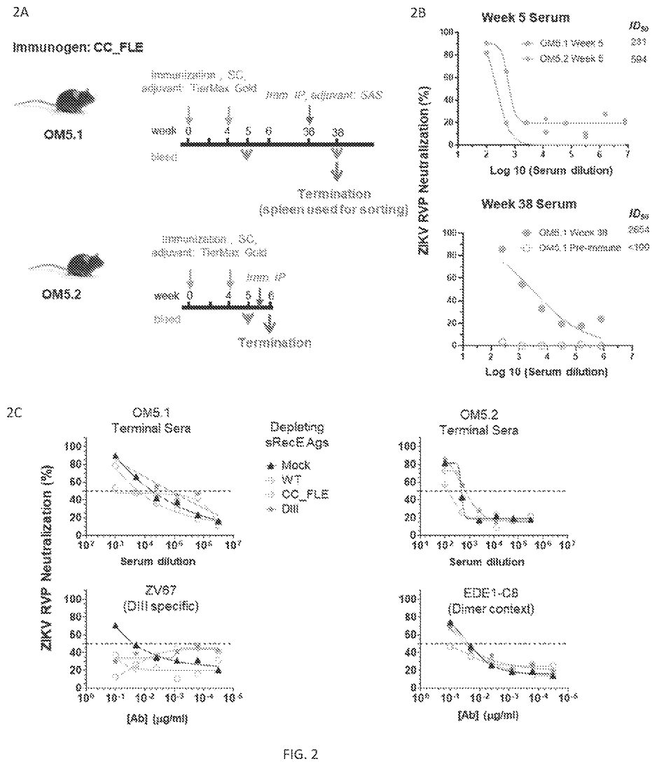

In some embodiments, a subject antibody comprises: a variable domain comprising: a) a heavy chain variable domain comprising: i. a CDR1 region that is identical in amino acid sequence to the sequence of a heavy chain CDR1 region of an antibody selected from the antibodies shown in FIGS. 1 and 2; ii. a CDR2 region that is identical in amino acid sequence to the sequence of a heavy chain CDR2 region of the selected antibody; and iii. a CDR3 region that is identical in amino acid sequence to the sequence of a heavy chain CDR3 region of the selected antibody; and b) a light chain variable domain comprising: i. a CDR1 region that is identical in amino acid sequence to the sequence of a light chain CDR1 region of the selected antibody; ii. a CDR2 region that is identical in amino acid sequence to that of a light chain CDR2 region of the selected antibody sequence; and iii. a CDR3 region that is identical in amino acid sequence to that of a light chain CDR3 region of the selected antibody; wherein the antibody specifically binds Zika virus E protein.

In certain embodiments, an antibody comprises: (a) a variable domain comprising: i. a CDR1 region that is identical in amino acid sequence to the heavy chain CDR1 region of an antibody selected from the antibodies shown in FIGS. 1 and 2; ii. a CDR2 region that is identical in amino acid sequence to the heavy chain CDR2 region of the selected antibody; and iii. a CDR3 region that is identical in amino acid sequence to the heavy chain CDR3 region of the selected antibody; and a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; or (b) a variant of the variable domain of part (a) that is otherwise identical to the variable domain of part (a) except for up to 10 (e.g., up to 9, 8, 7, 6, 5, 4, 3, 2, or 1) amino acid substitutions in the collective CDR regions of the variable domain of (a), wherein the antibody binds to Zika virus E protein.

In some embodiments, the antibody may contain only a heavy chain variable domain described herein. In these embodiments, the antibody may be a “heavy chain only” antibody.

The heavy and light chain sequences disclosed herein can be analyzed via lineage analysis in order obtain consensus sequences for the CDRs. Analysis of these sequences reveals which amino acid positions can tolerate amino acid substitutions and, as such, can be used to make variants of the present antibodies that have, for example, amino acid substitutions in the CDRs.

In some embodiments, a subject antibody (e.g., a subject antibody that specifically binds Zika virus E protein) may comprise: a) a light chain region comprising: i) one, two, or three complementarity determining regions (CDRs) from a light chain variable region sequence of a selected anti-Zika virus E protein antibody; and ii) a light chain framework region, e.g., a framework region from a human immunoglobulin light chain; and b) a heavy chain region comprising: i) one, two, or three CDRs from the heavy chain variable region sequence of a selected antibody; and ii) a heavy chain framework region, e.g., a framework region from a human immunoglobulin heavy chain.

A subject antibody can comprise a heavy chain variable region comprising an amino acid sequence that is 85%, 86%, 87%, 88%, 89%, 90%, 91%, 92%, 93%, 94%, 95%, 96%, 97%, 98% or 99% identical to a sequence set forth in SEQ ID NOS: 2, 6, 10, 14, 18, 22, 26, 30, 34, 38, 42, 46, 50, 54, 58, 62, 66, 70, 74, 78, 82, 86, 90, 94, 98, 102, 106, 110, 114. A subject antibody can comprise a heavy chain variable region comprising one, two, or three of the heavy chain complementarity determining regions (CDRs) of a selected anti-Zika virus E protein antibody.

A subject antibody can comprise a light chain variable region comprising an amino acid sequence that is 85%, 86%, 87%, 88%, 89%, 90%, 91%, 92%, 93%, 94%, 95%, 96%, 97%, 98% or 99% identical to the sequence set forth in SEQ ID NOS: 4, 8, 12, 16, 20, 24, 28, 32, 36, 40, 44, 48, 52, 56, 60, 64, 68, 72, 76, 80, 84, 88, 92, 96, 100, 104, 108, 112, 116. A subject antibody can comprise a light chain variable region comprising one, two, or three of the light chain CDRs of a selected anti-Zika virus E protein antibody.

In some embodiments, a subject antibody comprises anti-Zika virus E protein antibody heavy chain CDRs and anti-Zika virus E protein antibody light chain CDRs in a single polypeptide chain, e.g., in some embodiments, a subject antibody is a scFv. In some embodiments, a subject antibody comprises, in order from N-terminus to C-terminus: a first amino acid sequence of from about 5 amino acids to about 25 amino acids in length; an light chain CDR1 of a selected anti-Zika virus E protein antibody; a second amino acid sequence of from about 5 amino acids to about 25 amino acids in length; a light chain CDR2 of a selected anti-Zika virus E protein antibody; a third amino acid sequence of from about 5 amino acids to about 25 amino acids in length; a light chain CDR3 of a selected anti-Zika virus E protein antibody; a fourth amino acid sequence of from about 5 amino acids to about 25 amino acids in length; a heavy chain CDR1 of a selected anti-Zika virus E protein antibody; a fifth amino acid sequence of from about 5 amino acids to about 25 amino acids in length; a heavy chain CDR2 of a selected anti-Zika virus E protein antibody; a sixth amino acid sequence of from about 5 amino acids to about 25 amino acids in length; a heavy chain CDR3 of a selected anti-Zika virus E protein antibody; and a seventh amino acid sequence of from about 5 amino acids to about 25 amino acids in length. In some embodiments, a subject antibody may comprise, in order from N-terminus to C-terminus: a light chain FRI region; a light chain CDR1 of a selected anti-Zika virus E protein antibody; a light chain FR2 region; a light chain CDR2 of a selected anti-Zika virus E protein antibody; a light chain FR3 region; a light chain CDR3 of a selected anti-Zika virus E protein antibody; optionally a light chain FR4 region; a linker region; optionally a heavy chain FRI region; a heavy chain CDR1 of a selected anti-Zika virus E protein antibody; a heavy chain FR2 region; a heavy chain CDR2 of a selected anti-Zika virus E protein antibody; a heavy chain FR3 region; a heavy chain CDR3 of a selected anti-Zika virus E protein antibody; and a heavy chain FR4 region. In some of these embodiments, each of the FR regions is a human FR region. The linker region can be from about 5 amino acids (aa) to about 50 amino acids (aa) in length, e.g., from about 5 aa to about 10 aa, from about 10 aa to about 15 aa, from about 15 aa to about 20 aa, from about 20 aa to about 25 aa, from about 25 aa to about 30 aa, from about 30 aa to about 35 aa, from about 35 aa to about 40 aa, from about 40 aa to about 45 aa, or from about 45 aa to about 50 aa in length.

Linkers suitable for use a subject antibody include “flexible linkers”. If present, the linker molecules are generally of sufficient length to permit some flexible movement between linked regions. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, GSGGSₙ and GGGSₙ, where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGSG, GGSGG, GSGSG, GSGGG, GGGSG, GSSSG, and the like. The ordinarily skilled artisan will recognize that design of a peptide conjugated to any elements described above can include linkers that are ah or partially flexible, such that the linker can include a flexible linker as well as one or more portions that confer less flexible structure.

In some embodiments, a subject antibody may be “humanized.” The term “humanized antibody” refers to an antibody comprising at least one chain comprising variable region framework residues substantially from a human antibody chain (referred to as the acceptor immunoglobulin or antibody) and at least one CDR substantially from a non-human antibody (referred to as the donor immunoglobulin or antibody). See, Queen et al., Proc. Natl. Acad. Sci. USA 86:10029 10033 (1989), U.S. Pat. Nos. 5,530,101, 5,585,089, 5,693,761, WO 90/07861, and U.S. Pat. No. 5,225,539. The constant region(s), if present, can also be substantially or entirely from a human immunoglobulin. In some embodiments, a subject antibody comprises one or more of the CDRs and one or more FR regions from a human antibody. Methods of making humanized antibodies are known in the art. See, e.g., U.S. Pat. No. 7,256,273.

The substitution of mouse CDRs into a human variable domain framework can result in retention of their correct spatial orientation where, e.g., the human variable domain framework adopts the same or similar conformation to the mouse variable framework from which the CDRs originated. This can be achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies. See Kettleborough et al., Protein Engineering 4:773 (1991); Kolbinger et al., Protein Engineering 6:971 (1993).

Having identified the complementarity determining regions of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HAMA response in a human subject. Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity.

The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

CDR and framework regions are as defined by Kabat, Sequences of Proteins of Immunological Interest (National Institutes of Health, Bethesda, Md., 1987 and 1991). An alternative structural definition has been proposed by Chothia et al., J. Mol. Biol. 196:901 (1987); Nature 342:878 (1989); and J. Mol. Biol. 186:651 (1989) (collectively referred to as “Chothia”). When framework residues, as defined by Kabat, supra, constitute structural loop residues as defined by Chothia, supra, the amino acids present in the mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody.

In some embodiments, a subject antibody comprises scFv multimers. For example, in some embodiments, a subject antibody is an scFv dimer (e.g., comprises two tandem scFv (scFv2)), an scFv trimer (e.g., comprises three tandem scFv (scFv3)), an scFv tetramer (e.g., comprises four tandem scFv (scFvₜ), or is a multimer of more than four scFv (e.g., in tandem). The scFv monomers can be linked in tandem via linkers of from about 2 amino acids to about 10 amino acids in length, e.g., 2 aa, 3 aa, 4 aa, 5 aa, 6 aa, 7 aa, 8 aa, 9 aa, or 10 aa in length. Suitable linkers include, e.g., (Gly)ₓ, where x is an integer from 2 to 10. Other suitable linkers are those discussed above. In some embodiments, each of the scFv monomers in a subject scFV multimer is humanized, as described above.

In some embodiments, a subject antibody comprises a constant region of an immunoglobulin (e.g., an Fc region). The Fc region, if present, can be a human Fc region. If constant regions are present, the antibody can contain both light chain and heavy chain constant regions. Suitable heavy chain constant region include CHI, hinge, CH2, CH3, and CH4 regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. Light chain constant regions can be lambda or kappa. A subject antibody (e.g., a subject humanized antibody) can comprise sequences from more than one class or isotype. Antibodies can be expressed as tetramers containing two light and two heavy chains, as separate heavy chains, light chains, as Fab, Fab′ F(ab′)2, and Fv, or as single chain antibodies in which heavy and light chain variable domains are linked through a spacer.

In some embodiments, a subject antibody comprises a free thiol (—SH) group at the carboxyl terminus, where the free thiol group can be used to attach the antibody to a second polypeptide (e.g., another antibody, including a subject antibody), a scaffold, a carrier, etc.

### Antibody Discovery

Transgenic animal platforms for the discovery of human monoclonal antibodies have been developed in animals including mammals (e.g., mice, rats, rabbits, and cows, Brüggemann et al., Arch Immunol Ther Exp (Warsz) 2015 63 (2): 101-8) and birds (e.g., chickens, Ching et al., MAbs. 2018 January; 10 (1): 71-80). Non-animal systems such as phage or yeast display libraries are also known in the art and may also be used.

### Methods of Producing a Subject Antibody

A subject antibody can be produced by any known method, e.g., conventional synthetic methods for protein synthesis; recombinant DNA methods; etc.

Where a subject antibody is a single chain polypeptide, it can be synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85:2149-2156 (1963); Stewart et al., Solid Phase Peptide Synthesis, 2nd ed. Pierce Chem. Co., Rockford, Ill. (1984); and Ganesan A. 2006 Mini Rev. Med Chem. 6:3-10 and Camarero J A et al. 2005 Protein Pept Lett. 12:723-8. Briefly, small insoluble, porous beads are treated with functional units on which peptide chains are built. After repeated cycling of coupling/deprotection, the free N-terminal amine of a solid-phase attached is coupled to a single N-protected amino acid unit. This unit is then deprotected, revealing a new N-terminal amine to which a further amino acid may be attached. The peptide remains immobilized on the solid-phase and undergoes a filtration process before being cleaved off.

Standard recombinant methods can be used for production of a subject antibody. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the code, a variety of nucleic acid sequences can encode each immunoglobulin amino acid sequence. The desired nucleic acid sequences can be produced by de novo solid-phase DNA synthesis or by polymerase chain reaction (PCR) mutagenesis of an earlier prepared variant of the desired polynucleotide. Oligonucleotide-mediated mutagenesis is an example of a suitable method for preparing substitution, deletion and insertion variants of target polypeptide DNA. See Adelman et al., DNA 2:183 (1983). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression. Saccharomyces (e.g., S. cerevisiae) and Pichia are examples of suitable yeast host cells, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian cells (e.g., mammalian cells grown in in vitro cell culture) can also be used to express and produce the polypeptides of the present invention (e.g., polynucleotides encoding immunoglobulins or fragments thereof). See Winnacker, From Genes to Clones, VCH Publishers, N.Y., N.Y. (1987). Suitable mammalian host cells include CHO cell lines, various Cos cell lines, HeLa cells, myeloma cell lines, and transformed B-cells or hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like. See Co et al., J. Immunol. 148:1149 (1992).

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

### Production of Subject Antibodies in Transgenic Animals

Antibodies directed to Zika virus E protein were produced in OmniMouse® animals (PCTUS2018014568, OmniAb, Inc.). These mice have deletions of the JH and CK genes and are not able to produce endogenous immunoglobulins. They carry four transgenes consisting of the un-rearranged human antibody Ig κ and λ loci and recombinant transgenes comprising human VH, DH and JH with rat constant regions. OmniMouse animals express 44 functional VH, 20 functional VK and 15 functional VL, closely mimicking the naïve human B repertoire, enabling the characterization of human B cell repertoire in response to immunization in a preclinical setting. Other transgenic animal models for producing humanized antibodies are known in the art.

### Antibodies of the Invention Include Heavy Chain-Only Antibodies

In one embodiment, transgenic animals that lack a functional Ig light chain locus, and comprising at least two artificial heavy chain loci, are immunized with antigen to produce heavy chain-only antibodies that specifically bind to ZIKV E protein.

In one embodiment, the invention provides monoclonal antibody producing cells derived from such animals, as well as nucleic acids derived therefrom. Also provided are hybridomas derived therefrom. Also provided are fully human heavy chain-only antibodies, as well as encoding nucleic acids, derived therefrom.

Teachings on heavy chain-only antibodies are found in the art. For example, see PCT publications WO02085944, WO02085945, WO2006008548, and WO2007096779. See also U.S. Pat. Nos. 5,840,526; 5,874,541; 6,005,079; 6,765,087; 5,800,988; EP 1589107; WO 9734103; and U.S. Pat. No. 6,015,695.

### Pharmaceutical Compositions

In further embodiments, one or more antibodies, fragments thereof, polypeptides, or nucleic acids as described herein are admixed with an appropriate pharmaceutical carrier suitable for administration to subjects, so as to generate pharmaceutical compositions.

Subjects that receive the pharmaceutical compositions described herein are preferably mammals, more preferably humans, though veterinary uses are also contemplated.

Pharmaceutically acceptable carriers that can be employed in the pharmaceutical compositions described herein may comprise a solvent, dispersion media, or isotonic agent. Except insofar as any conventional media, agent, diluent or carrier is detrimental to the recipient or to the therapeutic effectiveness of the agents contained therein, its use in the pharmaceutical compositions of the present invention is appropriate.

The carrier can be liquid, semi-solid, e.g. pastes, or solid carriers. Examples of carriers include oils, water, saline solutions, alcohol, sugar, gel, lipids, liposomes, resins, porous matrices, binders, fillers, coatings, preservatives, or combinations thereof.

### Formulations and Pharmaceutical Compositions

Aspects of the invention include compositions, such as pharmaceutical compositions, comprising one or more antibodies, fragments thereof, polypeptides, or nucleic acids as described herein. In general, a formulation comprises an effective amount of a one or more antibodies, fragments thereof, polypeptides, or nucleic acids as described herein. An “effective amount” means a dosage sufficient to produce a desired result, e.g., reduction in weight or blood glucose. Generally, the desired result is at least a reduction in a symptom of a ZIKV-associated disorder or condition as compared to a control. The antibodies, fragments thereof, polypeptides, or nucleic acids as described herein can be delivered in such a manner as to avoid the blood-brain barrier, as described in more detail below. The antibodies, fragments thereof, polypeptides, or nucleic acids as described herein can be formulated and/or modified to enable crossing of the blood-brain barrier, if necessary. A treatment or therapy can involve administering the antibodies, fragments thereof, polypeptides, or nucleic acids as described herein to a patient already suffering from a disease thus causing a therapeutically beneficial effect, such as ameliorating existing symptoms, ameliorating the underlying causes of symptoms, postponing or preventing the further development of a disorder, and/or reducing the severity of symptoms that will or are expected to develop.

The antibodies, fragments thereof, polypeptides, or nucleic acids as described herein can be administered to the host using several approaches thereby resulting in the desired therapeutic effect or diagnostic effect. Thus, the antibodies, fragments thereof, polypeptides, or nucleic acids as described herein can be incorporated into a variety of formulations for therapeutic administration. More particularly, the antibodies, fragments thereof, polypeptides, or nucleic acids as described herein can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants or aerosols.

In pharmaceutical dosage forms, the antibodies, fragments thereof, polypeptides, or nucleic acids as described herein can be administered in the form of pharmaceutically acceptable salts, or they may also be used alone or in appropriate association, as well as in combination, with other pharmaceutically active compounds. The following methods and excipients are merely exemplary and are in no way limiting.

For oral preparations, the antibodies, fragments thereof, polypeptides, or nucleic acids as described herein can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives or flavoring agents.

The antibodies, fragments thereof, polypeptides, or nucleic acids as described herein can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers or preservatives.

Pharmaceutical compositions comprising the antibodies, fragments thereof, polypeptides, or nucleic acids as described herein are prepared by mixing the agent having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline or combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, or neuraminic acid; and/or non-ionic surfactants such as Tween, Brij Pluronics, Triton-X, or polyethylene glycol (PEG).

The pharmaceutical composition comprising the one or more antibodies, fragments thereof, polypeptides, or nucleic acids as described herein may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration; see also Chen (1992) Drug Dev Ind Pharm 18, 1311-54.

Exemplary concentrations in a subject receiving one or more of the aforementioned pharmaceutical compositions may range from about 1 mg/mL to about 200 mg/ml or from about 50 mg/mL to about 200 mg/mL, or from about 150 mg/mL to about 200 mg/mL.

An aqueous formulation of the antibodies, fragments thereof, polypeptides, or nucleic acids as described herein may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation comprising the antibodies, fragments thereof, polypeptides, or nucleic acids as described herein to modulate the tonicity of the formulation. Exemplary tonicity agents include sodium chloride, potassium chloride, glycerin or any component from the group of amino acids, sugars, as well as, combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A surfactant may also be added to the formulation comprising the antibodies, fragments thereof, polypeptides, or nucleic acids as described herein to reduce aggregation and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), or sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™.

Examples of suitable Polyoxyethylene alkyl ethers are those sold under the trademark Brij™. Exemplary concentrations of surfactant may range from about 0.001% to about 1% w/v.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol or glycerol); or amino acids (including alanine, glycine or glutamic acid). Lyoprotectants can be included in an amount of about 10 mM to 500 nM.

In some embodiments, a subject formulation includes one or more antibodies, fragments thereof, polypeptides, or nucleic acids as described herein, and one or more of the above-identified agents (e.g., a surfactant, a buffer, a stabilizer, or a tonicity agent) and, optionally comprises one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a subject formulation can be a liquid or lyophilized formulation comprising one or more of the antibodies, fragments thereof, polypeptides, or nucleic acids as described herein suitable for parenteral administration, and can comprise: about 1 mg/mL to about 200 mg/mL of one or more of the antibodies, fragments thereof, polypeptides, or nucleic acids as described herein; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0. As another example, a subject parenteral formulation is a liquid or lyophilized formulation comprising: about 1 mg/mL to about 200 mg/mL of one or more of the antibodies, fragments thereof, polypeptides, or nucleic acids as described herein; 0.04% Tween 20 w/v; 20 mM L-histidine; and 250 mM Sucrose; and has a pH of 5.5.

As another example, a subject parenteral formulation comprises a lyophilized formulation comprising: 1) 15 mg/mL of one or more of the antibodies, fragments thereof, polypeptides, or nucleic acids as described herein; 0.04% Tween 20 w/v; 20 mM L-histidine; and 250 mM sucrose; and has a pH of 5.5; or 2) 75 mg/mL of one or more of the antibodies, fragments thereof, polypeptides, or nucleic acids as described herein; 0.04% Tween 20 w/v; 20 mM L-histidine; and 250 mM sucrose; and has a pH of 5.5; or 3) 75 mg/mL of one or more of the antibodies, fragments thereof, polypeptides, or nucleic acids as described herein; 0.02% Tween 20 w/v; 20 mM L-histidine; and 250 mM Sucrose; and has a pH of 5.5; or 4) 75 mg/mL of a subject antibody; 0.04% Tween 20 w/v; 20 mM L-histidine; and 250 mM trehalose; and has a pH of 5.5; or 6) 75 mg/mL of one or more of the antibodies, fragments thereof, polypeptides, or nucleic acids as described herein; 0.02% Tween 20 w/v; 20 mM L-histidine; and 250 mM trehalose; and has a pH of 5.5.

As another example, a subject parenteral formulation is a liquid formulation comprising: 1) 7.5 mg/mL of one or more of the antibodies, fragments thereof, polypeptides, or nucleic acids as described herein; 0.022% Tween 20 w/v; 120 mM L-histidine; and 250 125 mM sucrose; and has a pH of 5.5; or 2) 37.5 mg/mL of one or more of the antibodies, fragments thereof, polypeptides, or nucleic acids as described herein; 0.02% Tween 20 w/v; 10 mM L-histidine; and 125 mM sucrose; and has a pH of 5.5; or 3) 37.5 mg/mL of one or more of the antibodies, fragments thereof, polypeptides, or nucleic acids as described herein; 0.01% Tween 20 w/v; 10 mM L-histidine; and 125 mM sucrose; and has a pH of 5.5; or 4) 37.5 mg/mL of one or more of the antibodies, fragments thereof, polypeptides, or nucleic acids as described herein; 0.02% Tween 20 w/v; 10 mM L-histidine; 125 mM trehalose; and has a pH of 5.5; or 5) 37.5 mg/ml of one or more of the antibodies, fragments thereof, polypeptides, or nucleic acids as described herein; 0.01% Tween 20 w/v; 10 mM L-histidine; and 125 mM trehalose; and has a pH of 5.5; or 6) 5 mg/mL of one or more of the antibodies, fragments thereof, polypeptides, or nucleic acids as described herein; 0.02% Tween 20 w/v; 20 mM L-histidine; and 250 mM trehalose; and has a pH of 5.5; or 7) 75 mg/mL of one or more of the antibodies, fragments thereof, polypeptides, or nucleic acids as described herein; 0.02% Tween 20 w/v; 20 mM L-histidine; and 250 mM mannitol; and has a pH of 5.5; or 8) 75 mg/mL of one or more of the antibodies, fragments thereof, polypeptides, or nucleic acids as described herein; 0.02% Tween 20 w/v; 20 mM L histidine; and 140 mM sodium chloride; and has a pH of 5.5; or 9) 150 mg/mL of one or more of the antibodies, fragments thereof, polypeptides, or nucleic acids as described herein; 0.02% Tween 20 w/v; 20 mM L-histidine; and 250 mM trehalose; and has a pH of 5.5; or 10) 150 mg/mL of a subject antibody; 0.02% Tween 20 w/v; 20 mM L-histidine; and 250 mM mannitol; and has a pH of 5.5; or 11) 150 mg/mL of one or more of the antibodies, fragments thereof, polypeptides, or nucleic acids as described herein; 0.02% Tween 20 w/v; 20 mM L-histidine; and 140 mM sodium chloride; and has a pH of 5.5; or 12) 10 mg/mL of one or more of the antibodies, fragments thereof, polypeptides, or nucleic acids as described herein; 0.01% Tween 20 w/v; 20 mM L-histidine; and 40 mM sodium chloride; and has a pH of 5.5.

The one or more of the antibodies, fragments thereof, polypeptides, or nucleic acids as described herein can be utilized in an aerosol formulation to be administered via inhalation. The one or more of the antibodies, fragments thereof, polypeptides, or nucleic acids as described herein can be formulated into pressurized acceptable propellants such as dichlorodifluoromethane, propane, or nitrogen. In some embodiments, one or more of the nucleic acids described herein are provided to a subject as a nucleic acid immunogen or vaccine such as by injection with or without electroporation.

Furthermore, the one or more of the antibodies, fragments thereof, polypeptides, or nucleic acids as described herein can be made into suppositories by mixing with a variety of bases such as emulsifying bases or water-soluble bases. The one or more of the antibodies, fragments thereof, polypeptides, or nucleic acids as described herein can then be administered rectally via a suppository. The suppository can include vehicles such as cocoa butter, carbowaxes or polyethylene glycols, which melt at body temperature, yet are solidified at room temperature. Unit dosage form for oral or rectal administration such as syrups, elixirs, or suspensions may be provided, wherein each dosage unit, for example, teaspoonful, tablespoonful, tablet or suppository, contains a predetermined amount of the composition containing the one or more of the antibodies, fragments thereof, polypeptides, or nucleic acids as described herein. Similarly, unit dosage forms for injection or intravenous administration may comprise the one or more antibodies, fragments thereof, polypeptides, or nucleic acids as described herein in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of the one or more antibodies, fragments thereof, polypeptides, or nucleic acids as described herein calculated in an amount sufficient to produce the desired effect in association with a pharmaceutically acceptable diluent, carrier or vehicle. The specifications for the one or more antibodies, fragments thereof, polypeptides, or nucleic acids as described herein may depend on the particular agent employed and the effect to be achieved, and the pharmacodynamics associated with each antibody in the host.

Other modes of administration will also find use with the subject invention. For instance, the one or more antibodies, fragments thereof, polypeptides, or nucleic acids as described herein can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol or benzalkonium chloride. A surfactant may be present to enhance absorption of the one or more antibodies, fragments thereof, polypeptides, or nucleic acids as described herein by the nasal mucosa.

The one or more antibodies, fragments thereof, polypeptides, or nucleic acids as described herein can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the one or more antibodies, fragments thereof, polypeptides, or nucleic acids as described herein encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pFl buffering agents. Actual methods of preparing such dosage forms are known, or will be apparent, to those skilled in the art. See, e.g., Remington's Pharmaceutical Sciences, Mack Publishing Company, Easton, Pennsylvania, 17th edition, 1985. The composition or formulation to be administered will, in any event, contain a quantity of a subject antibody adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pFl adjusting and buffering agents, tonicity adjusting agents, stabilizers, wetting agents and the like, are readily available to the public.

In some embodiments, the one or more antibodies, fragments thereof, polypeptides, or nucleic acids as described herein is formulated in a controlled release formulation. Sustained-release preparations may be prepared using methods well known in the art. Suitable examples of sustained-release preparations include semipermeable matrices of solid hydrophobic polymers containing the antibody in which the matrices are in the form of shaped articles, e.g. films or microcapsules. Examples of sustained-release matrices include polyesters, copolymers of L-glutamic acid or ethyl-L-glutamate, non-degradable ethylene-vinyl acetate, hydrogels, polylactides, degradable lactic acid-glycolic acid copolymers or poly-D-(−)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, or extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.)

There are a number of controlled release drug formulations comprising the one or more antibodies, fragments thereof, polypeptides, or nucleic acids as described herein that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic or anionic drugs. Additional information regarding controlled release drug delivery systems may be found in Yie W. Chien, Novel Drug Delivery Systems, 1992 (Marcel Dekker, Inc.). Some of these formulations are discussed herein.

### Dosages

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. The one or more antibodies, fragments thereof, polypeptides, or nucleic acids as described herein may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 pg to 10 mg per kilogram of body weight per minute.

Those of skill will readily appreciate that dose levels can vary as a function of the specific agent, the severity of the symptoms and the susceptibility of the subject to side effects. Preferred dosages for a given agent are readily determinable by those of skill in the art by a variety of means.

### Routes of Administration

The one or more antibodies, fragments thereof, polypeptides, or nucleic acids as described herein can be administered to a subject using any available method and route suitable for drug delivery, including in vivo and ex vivo methods, as well as systemic and localized routes of administration, including electroporation.

In some embodiments, the antibody or fragment thereof can be administered to the patient as an intravenous infusion (200 mg or 2 mg/kg, up to 200 mg) over 10-60 (e.g., 30 minutes), every 2-4 (e.g., three) weeks in a pharmaceutically acceptable carrier, e.g., PBS.

Conventional and pharmaceutically acceptable routes of administration include intranasal, intramuscular, intratracheal, subcutaneous, intradermal, topical application, intravenous, intraarterial, rectal, nasal, oral, and other enteral or parenteral routes of administration. Routes of administration may be combined, if desired, or adjusted depending upon the antibody and/or the desired effect. The one or more antibodies, fragments thereof, polypeptides, or nucleic acids as described herein can be administered in a single dose or in multiple doses. In some embodiments, the one or more antibodies, fragments thereof, polypeptides, or nucleic acids as described herein is administered orally. In some embodiments, the one or more antibodies, fragments thereof, polypeptides, or nucleic acids as described herein is administered via an inhalational route. In some embodiments, the one or more antibodies, fragments thereof, polypeptides, or nucleic acids as described herein is administered intranasally. In some embodiments, the one or more antibodies, fragments thereof, polypeptides, or nucleic acids as described herein is administered locally. In some embodiments, the one or more antibodies, fragments thereof, polypeptides, or nucleic acids as described herein is administered intracranially. In some embodiments, the one or more antibodies, fragments thereof, polypeptides, or nucleic acids as described herein composition is administered intravenously. In some embodiments, the one or more antibodies, fragments thereof, polypeptides, or nucleic acids as described herein is administered by injection followed by electroporation.

Parenteral routes of administration of the one or more antibodies, fragments thereof, polypeptides, or nucleic acids as described herein other than inhalation administration include, but are not necessarily limited to, topical, transdermal, subcutaneous, intramuscular, intraorbital, intracapsular, intraspinal, intrasternal, and intravenous routes, e.g., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

The one or more antibodies, fragments thereof, polypeptides, or nucleic acids as described herein can also be delivered to the subject by enteral administration. Enteral routes of administration include, but are not necessarily limited to, oral and rectal (e.g., using a suppository) delivery, as described above.

Kits with unit doses of the one or more antibodies, fragments thereof, polypeptides, or nucleic acids as described herein, e.g. in oral or injectable doses, are provided. In such kits, in addition to the containers containing the unit doses will be an informational package insert describing the use and attendant benefits of the agent to be delivered in treating, inhibiting, or preventing Zika infection.

### General

In some embodiments, the one or more antibodies, fragments thereof, or polypeptides as described herein binds and/or neutralizes ZIKV and in some embodiments the one or more nucleic acids described herein encode a polypeptide, which binds and/or neutralizes ZIKV. In some of the aforementioned embodiments, the antibody isotype is IgG1, IgG2, IgG3, IgG4, IgA1, IgA2, or IgM. Additional embodiments include a composition that comprises one or more antibodies, fragments thereof, polypeptides, or nucleic acids as described herein and in some embodiments, the antibody format is an ScFv, heavy chain only, or VHH. In some embodiments, the nucleic acid composition comprises DNA, RNA, or pRNA. In some embodiments, the nucleic acid composition is formulated for administration to a subject, such as a human subject.

In some embodiments, the antibodies, binding fragments thereof, or polypeptides target the quaternary epitope of the ZIKV E protein. In some embodiments, the antibodies, binding fragments thereof, or polypeptides target the domain III epitope of the ZIKV E protein.

In some embodiments, the epitope specificity of one or more of the monoclonal antibodies described herein is determined by biolayer light interferometry (BLI). In some embodiments, BLI is measured using an Octet RED96 instrument and Octet Analysis software. In some embodiments, the ability of one or more antibodies, fragments thereof, or polypeptides to neutralize ZIKV infection was determined. In some embodiments, this assessment was performed by neutralization assay with ZIKV reporter virus particles (RVPs).

In some embodiments, a structure determination of the generated antibody was determined by single particle reconstruction of the antibody-sRecE_CC core complex using cryoelectron microscopy (cryoEM). In some embodiments, CC_Core is a dimeric variant of ZIKV E protein bearing mutation A264C.

### Example 1

### Methods

### Biolayer Interferometry

Biolayer light interferometry (BLI) was performed using an Octet RED96 instrument (ForteBio, Pall Life Sciences) as described previously (17, 18). Antibody was captured onto anti-human Fc biosensors at concentration of 10 μg/ml as ligand and the tested samples of E proteins was diluted in 3×2-fold series starting from 250 nM to 62.5 nM in solution, respectively unless otherwise indicated. Briefly, biosensors, pre-hydrated in binding buffer (1×PBS, 0.01% BSA and 0.2% Tween-20) for 10 min were first immersed in binding buffer for 60 s to establish a baseline followed by submerging in a solution containing ligand for 60 s to capture ligand. The biosensors were then submerged in binding buffer for a wash for 60 s. The biosensors were then immersed in a solution containing various concentrations of tested samples as analyte for 120 s to detect analyte/ligand association, followed by 120 s in binding buffer to assess analyte/ligand dissociation. Binding affinity constants (dissociation constant, K_{D}; on-rate, kₒₙ; off-rate, k_{off}) were determined using Octet Analysis software.

### Animal Experiments

For the immunogenicity study with the transgenic mice that express naïve human Ig loci, two 20-to-21-week old female transgenic mice, designated as OM5.1 and OM5.2, were subcutaneously (SC) immunized with 20 μg of sRecE-CC_FLE formulated in TiterMax Gold (Sigma) adjuvant on weeks 0, and 4. Subsequently, mouse OM5.1 received an intraperitoneal (IP) immunization on week 36 with 20 μg of sRecE-CC_FLE formulated in Sigma Adjuvant System (Sigma) and terminated on week 38. Mouse OM5.2 received additional IP immunization with 20 μg of sRecE-CC_FLE without any adjuvant, four days before termination on week 6.

### ZIKV Reporter Virus Particle (RVP) Construction, Production and Neutralization Assay

As previously described, to quantitate ZIKV mAb neutralization capacity and neutralizing antibody titers in serum samples, a high throughput plate based ZIKV reporter virus particle (RVP) assay using a plasmid encoding ZIKV envelope proteins and capsid (pZIKV/HPF/CprME, ZIKV French Polynesian strain H/PF/2013) and another West Nile virus (WNV) subgenomic replicon-expressing plasmid pWNVII-Rep-REN-IB carrying reporter gene (Renilla luciferase) were utilized to produce ZIKV RVP. Briefly, following the co-transfection of plasmids pWNVII-Rep-REN-IB and pZIKV/HPF/CprME into 293T cells, RVP-containing supernatants were harvested after incubation for 48-72 hr at 30 °C, filtered through a 0.45-micron filter and frozen at −80 °C. RVPs were titrated on Vero cells with duplicate to determine the dilution to use in the RVP-based neutralization assay to achieve ~1-5×10⁴ RLU in the absence of serum/antibody, which is 1,000-fold higher than background (cells only).

In the neutralization assays, the heat-inactivated serum starting from 100-fold dilution with serial dilutions was incubated with diluted RVP in EMEM/10% FBS for 1 hour at 37 °C before infecting Vero cells at 37 °C, 5% CO₂ for 1 hour. The next day, cells were lysed with lysis buffer (Promega) for 40 minutes at room temperature with shaking prior to the addition of Renilla Luciferase Assay System (Promega) according to the manufacturer's instruction. The luminesce was read immediately on a Molecular Devices reader. Percent neutralization was calculated based on wells containing virus only and cells only as background. Data was fit to a 4PL curve in GraphPad Prism 8. ID₅₀ values represented the reciprocal of the serum dilution that resulted in 50% inhibition compared to RVP alone.

For the serum neutralizing depletion assay, a dilution of pooled sera was pre-mixed with various E variant proteins at concentration of 0.4 μg/ml for 0.5 hour at 37 °C prior to RVP incubation. A dilution of antibodies (ZV67 and EDE1-C8) starting from 0.1 μg/ml was used as control antibodies. The mixture of RVP with serum or antibody depleted by soluble protein was added to Vero cells subsequently to quantify RVP entry signal (luciferase activity) as described above.

### Flow Cytometry & Ag-Specific B Cell Sorting

Cryopreserved transgenic animal splenocytes were thawed quickly in 37 °C water bath followed by treatment with DNaseI (10,000 U/ml, Roche) in RPMI 1640 with 10% FBS at room temperature for five minutes and washed with chilled PBS. After stained with Aqua Dead Cell Staining dye (Life Technologies) in PBS for 15 minutes, followed by two steps of staining procedure with each lasts for one hour. PBS was used to wash cells between these two staining procedures. Since the transgenic mice carry rat Ig constant region encoding sequences adjacent to the human heavy chain variable gene segments, most of the class-switched B cells would bear rat IgG constant region, the 1^{st} staining medium contains polyclonal goat anti-rat IgG PE/Dazzle 594 conjugate (Biolegend) to identify class-switched B cells. The 2^{nd} staining medium contains a cocktail of antibodies and antigen for identifying antigen-specific B cells essentially as described previously with minor modifications (19, 20). Briefly, a cocktail of antibodies diluted in RPMI 1640/10% FBS, which contains CD3 (clone 17A2), F4/80 (clone BM8), Gr1 (clone RB6-8C5) (PerCp/Cy5.5; BioLegend), CD19 (clone 1D3) (APC-Cy7; BD Pharmingen), B220 (clone RA3-6B2) (Alexa Fluor® 700; BioLegend), mouse IgG2a (clone RMG2a-62, FITC, Biolegend), IgG2b (clone R12-3), & 1 (clone A85-1) (FITC; BD Pharmingen), IgD (clone RA3-6B2) (Pacific Blue; BioLegend), and CC_FLE-streptavidin-PE conjugate was used to stain the cells. The stained cells were applied to a BD FACS Aria III cell sorter (BD Biosciences) to sort for antigen-specific class-switched B cells using the phenotype of CD19⁺/B220⁺/CD3⁻Gr1⁻F4/80⁻/mouse IgD⁻IgG⁻/rat IgG⁺/CC_FLE⁺ into 96-well plates at single-cell density, followed by single-cell reverse transcription and PCR reactions to amplify human Ig V (D) J gene segments as previously described (21, 22, 23).

The resultant human Ig gene segments were analyzed by IMGT/V-Quest using human Ig germline reference database, with the variable domains of selected heavy/light (VH/VL) chain matched clones being cloned into corresponding expression vectors, described previously (21), followed by co-transfection of VH/VL expression plasmids into 293F cells (24) to reconstitute human antibodies in IgG1 format. The expressed human antibodies were purified by Protein A Sepharose columns (GE Healthcare). To produce antibody Fab fragment, the heavy chain variable domain was cloned into Fab expression vector described previously (25), followed by co-transfection with light chain expression vector in 293F cells and purification using complete His-Tag Purification Resin (Roche).

### Example 2

### High-Resolution Analysis of CC_FLE-Elicited Antibody/B Cell Response

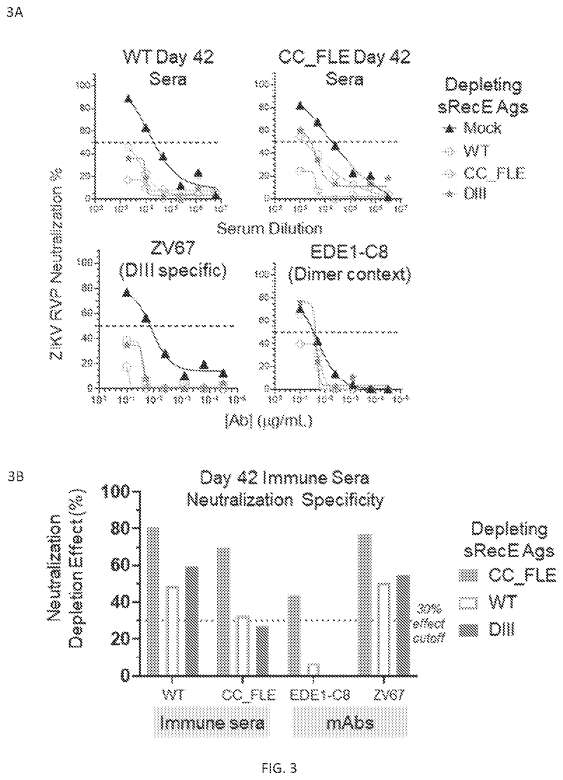

To delineate the nAb response elicited by CC_FLE at high resolution, the transgenic rodent was used. These transgenic mice carry un-rearranged human antibody loci, which closely mimics the naïve human B repertoire, enabling the characterization of human B cell repertoire in response to immunization in preclinical setting. Two transgenic mice were immunized with CC_FLE formulated in Sigma Adjuvant System for two and three times, respectively (FIG. 1A). After the 2^{nd} and 3^{rd} immunizations, mouse sera showed potent neutralization ID₅₀ titer against pseudotype ZIKV H/PF/2013 around 800 and 2,000, respectively (FIG. 2A-B). The transgenic immune sera neutralization specificity mapping pattern were similar to dimer-context specific mAb EDE1-C8 (FIG. 1B, FIG. 2B), and CC_FLE immunized C57BL/6 mice (FIG. 3A-B). The immune mice were then sacrificed and FACS-based antigen-specific single B cell sorting and cloning methodology was used to isolate CC_FLE binding mAbs from mouse splenocytes.

In an initial attempt, using CC_FLE-PE conjugate as antigen probe, CC_FLE binding memory B cells were sorted at single cell density (FIG. 1C), and Ig heavy (HC) and light chain (LC) encoding genes of each cell were amplified. 29 cells with both HC and LC amplified were obtained, from which four individual cells were randomly picked with matched HC/LC to express full-length mAbs in human IgG1 form for functional analysis. As expected, these four mAbs, designated as D4, B11, A1, and B9 respectively (Table 2), all bind dimeric antigen CC_FLE well (FIG. 1D), However, D4 and B11 bind monomeric WT sRecE poorly (FIG. 1D), similar to quaternary EDE mAb, EDE1-C8, suggesting dimer-context specificity. Moreover, A1 binds WT sRecE well but shows no binding to DIII, while B9 binds both WT sE and DIII, similar to DIII LR mAb Z004 (FIG. 1D). None of these mAbs binds E proteins of DENV 1 or 2 (data not shown), suggesting no cross-reactivity.

TABLE 2
Heavy Chain

| | VDJ gene usage | | | HCDR3-IMGT | | %SHM | |
|---|---|---|---|---|---|---|---|
| mAb ID | VH | D | JH | Sequence | Length | nt | AA |
| OZ-A1 | 4.34 | 3 | 6 | ARGGYYYGSGSSL | 22 | 2.8 | 3.1 |
| | | | | YYYYSGMDV | | | |
| OZ-89 | 3.43 | 5 | 4 | AKDIGYGGYGGFDY | 14 | 4.5 | 9.1 |
| OZ-811 | 4.31 | 3 | 4 | ARANYDILTGFDY | 13 | 1.4 | 3.0 |
| OZ-D4 | 4.59 | 6 | 3 | ARYSSGWSFSFDI | 13 | 0.0 | 0.0 |

Light Chain

| VJ gene usage | | | LCDR3-IMGT | | %SHM | | |
|---|---|---|---|---|---|---|---|
| VL | VK | J | Sequence | Length | nt | AA | Specificity |
| | 3.15 | 4 | QQYNNWPLT | 9 | 0.7 | 0.0 | Unknown |
| | 1.9 | 2 | QQLNSYPYT | 9 | 0.4 | 1.0 | Domain III |
| | 3.15 | 4 | QQYNNWPFT | 9 | 0.0 | 0.0 | E-dimer |
| 2.23 | | 3 | SSYAGGNTLV | 10 | 2.8 | 8.1 | E-dimer |

Furthermore, to map the epitope specificity, a BLI-based Ab competition assay was performed to evaluate the competition between these 4 mAbs and a panel of ZIKV reference mAbs (FIG. 1E). Based on mutual competition effect, it was found that these 4 mAbs belong to two competition clusters (FIG. 1E): (i) D4 and B11 compete mutually and compete with EDE mAbs, and (ii) A1 and B9 compete mutually and compete with DIII CC-loop mAb, ZV64, while B9 also competes with DIII LR mAb Z004. Therefore, D4 and B11 likely bind quaternary epitope EDE, the epitope of B9 overlaps with the DIII LR and DIII CC-loop. Finally, it was found that these CC_FLE-elicited mAbs all neutralize ZIKV pseudotype virus (FIG. 1F). D4 showed IC₅₀ titer at 0.048 μg/ml, as potent as EDE1-C8, while B9 has potency (IC₅₀=0.031 μg/ml) similar to DIII LR mAb, ZV67 (FIG. 1F).

Therefore, in transgenic mice that express naive human Ig loci, the lead immunogen, CC_FLE is able to elicit potent neutralizing antibody responses targeting the most prominent nAb epitopes including the quaternary EDE and DIII LR epitopes (Table 2).

### Example 3

Structure Determination of Novel ZIKV nAb Isolated from Transgenic Animal

CryoEM sample preparation, data collection, model refinement and analysis.

OZ-D4 Fab was incubated with CC_Core in a 1.5:1 molar ratio (per E protomer) overnight at 4 °C. The concentration of the complex was adjusted to 1 mg/ml in PBS, and 3 μl was added to a Quantifoil grid (R2/2 Cu 200 mesh or R1.2/1.3 Cu 300 mesh; Electron Microscopy Services) that had been freshly glow-discharged using a PELCO easiGLOW (Ted Pella). Samples were vitrified in 100% liquid ethane using a Mark IV Vitrobot (Thermo Fisher) at 100% humidity.

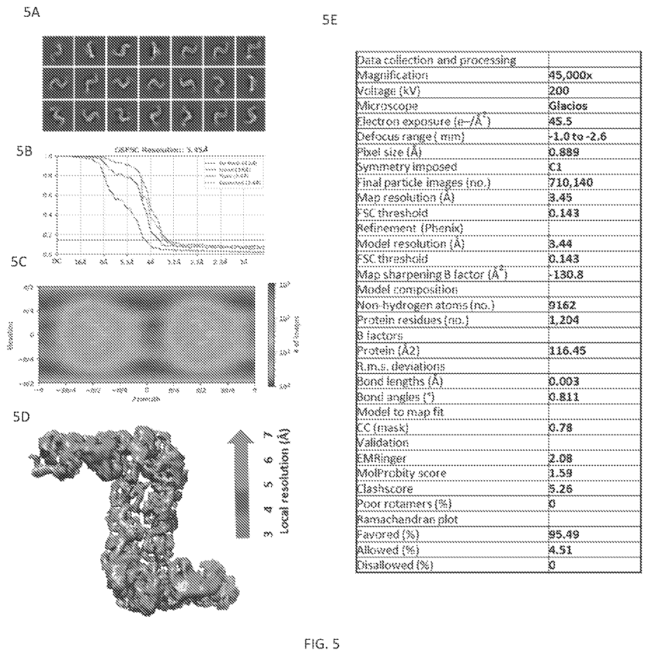

The micrographs were collected on a Glacios transmission electron microscope operating at 200 kV using EPU automated software (Thermo Fisher). Movies were obtained on a Gatan K3 direct electron detector at a nominal magnification of 45,000× using a defocus range of −1.0 and −2.5 μm. The micrographs were processed with cryoSPARC version 3.3.0 (36) and the final local 3D refinement of 710,140 particles with imposed Cl symmetry resulted in a 3.45 Å resolution map (FIG. 5).

The structure of ZIKV E dimer (pdb:5lbv) was used as initial template for CC_Core. OZ-D4 Fab variable region was generated with SAbPred (26). The individual chains of the initial models were docked into CryoEM density maps with UCSF Chimera (27), followed by the iterative cycles of building and refinement with Coot (28), Rosetta (29) and Phenix (Afonine et al., 2018). The structure visualization were performed using Chimera, ChimeraX (30) or Pymol (PyMOL Molecular Graphics System, Version 1.8 Schrödinger, LLC). Buried surface areas (BSAs), residue contacts, distances and crossing angles were calculated using the PDBePISA server (31) or Chimera (32).

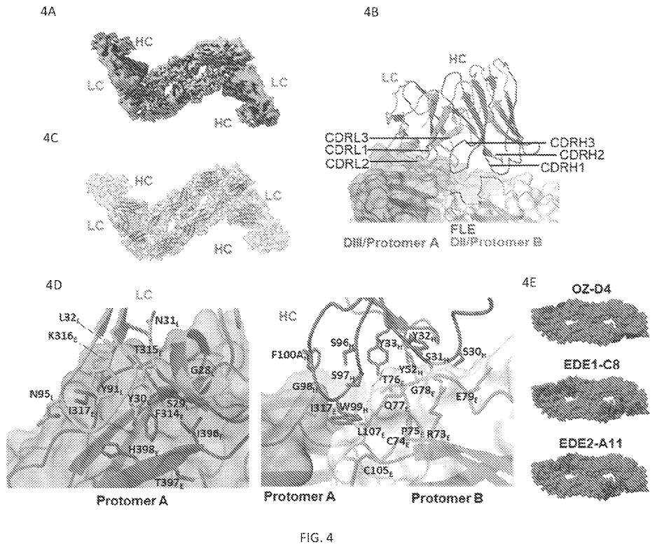

The structures were solved for selected nAbs (FIG. 1) isolated from transgenic mice complexed with the CC_Core, a dimeric variant of ZIKV E protein bearing mutation A264C by cryoelectron microscopy (cryoEM) single particle reconstruction. A 3.45 Å resolution structure was obtained for OZ-D4: ZIKV sRecE CC_Core complex (FIGS. 4A & 4B, FIG. 5), showing one ZIKV E dimer in a single conformation decorated by two OZ-D4 Fab fragments that bind E dimer quaternary epitope sites encompassing DII and DIII (FIG. 4B), partially overlapping with the reference mAb EDE1-C8. Overall, OZ-D4 binds to DIII of protomer A and DII of protomer B including part of FLE (FIG. 4C). The OZ-D4 epitope residues on E dimer include: (i) light chain contact residue clusters 313-318 (F314, T315, I317) and 396-398 (I396, H398) of DIII of promoter A (FIG. 4D, left), (ii) heavy chain contact residues including the end of β-strand b and adjacent loop residues 73-79 and fusion loop residues G106 and L107 (with 80 Å² buried surface area). on DII of protomer B, and I317 on DIII of protomer A (FIG. 4D, right). Note that hydrophobic residue I317 is engaged in both OZ-D4 heavy/light chain interaction accounting for nearly 18% of E dimer/OZ-D4 interface (FIG. 4D), while the other prominent hydrophobic contact residue F314 contributes to 20% of such interface by engaging OZ-D4 light chain (FIG. 4D).

OZ-D4 CDR-L1, CDR-L3, and CDR-H3 make the largest contribution to contact surface area with the ZIKV E dimer. Light chain residues Y30_{L} and Y91_{L} and interact with F314 and I317, respectively (FIG. 4D, left), while W99H on OZ-D4 CDR-H3 is involved in hydrophobic interaction with E dimer residues I317 and L107 at the E protomer interface (FIG. 4D, right). In summary, these data indicate that OZ-D4 has the major features of dimer context dependent EDE-class neutralizing mAb.

However, difference in epitope location between OZ-D4 and classical EDE mAbs including EDE1-C8 and EDE2-A11 is well appreciated (FIG. 4E). Particularly, OZ-D4 contacting residues 396-398 of Zika E are divergent from the corresponding residues on DENV E protein and are absent in the epitopes of EDE1-C8 and EDE2-All (FIG. 4E), consistent with the observation that OZ-D4 is not cross-reactive with DENV-1 and DENV-2 E protein. Note that classical EDE mAbs were isolated from DENV infected individual. Therefore, to our knowledge, OZ-D4 is the first immunization-elicited nAb (encoded by human Ig gene segments) targeting the quaternary neutralizing epitope of ZIKV E protein investigated at atomic resolution.

As expected, immune sera neutralization specificity mapping (FIG. 4) and antigen-specific single B cell sorting and monoclonal antibody cloning analysis (FIG. 4) revealed that dimeric CC_FLE is able to elicit potent nAb responses targeting multiple eminent epitopes such as the quaternary EDE and DIII LR epitopes, in contrast to the nAb responses elicited by monomeric sREcE-WT that are limited to DIII epitopes (FIG. 4). These features render the CC_FLE immunogen a more effective and safer ZIKV vaccine candidate than the nascent ZIKV E-based vaccine.

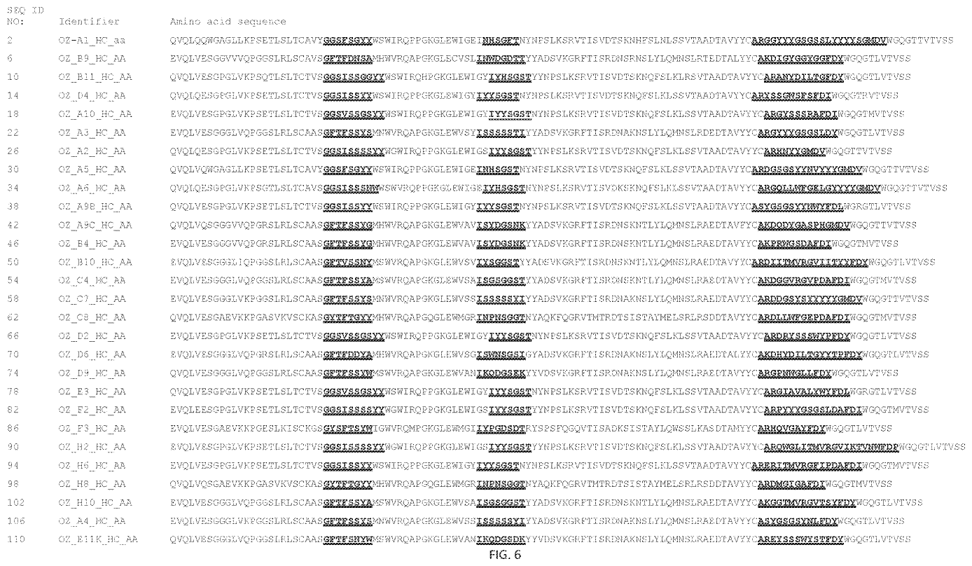

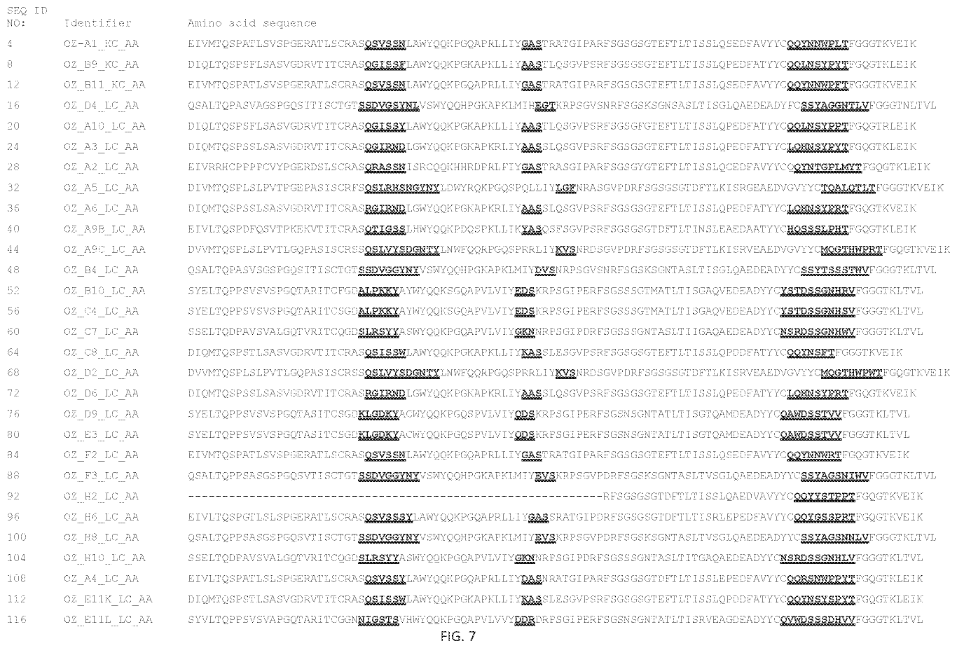

FIG. 6 comprises variable heavy chain sequences with the CDR1, 2, and 3 sequences appearing in order for each variable heavy chain sequence. The CDRs are indicated by bold and underlined text. FIG. 7 comprises variable light chain sequences with the CDR1, 2, and 3 sequences appearing in order for each variable light chain sequence except SEQ ID NO: 92 which only possess a LCDR3. The CDRs are indicated by bold and underlined text.

### FURTHER EMBODIMENTS OF THE INVENTION

Aspects of the invention additionally comprise the following numbered embodiments:

1. A polypeptide that binds to the E protein of Zika virus and comprises three immunoglobulin light chain CDR sequences, one or more of which differs by no more than one amino acid from the CDR1 sequence of SEQ ID NO: 204, the CDR2 sequence of GAS, and the CDR3 sequence of SEQ ID NO: 206.

2. A polypeptide that binds to the E protein of Zika virus and comprises three immunoglobulin heavy chain CDR sequences, one or more of which differs by no more than one amino acid from the CDR1 sequence of SEQ ID NO: 117, the CDR2 sequence of SEQ ID NO: 118, and the CDR3 sequence of SEQ ID NO: 119.

3. One or more polypeptides that bind to the E protein of Zika virus and together comprise six immunoglobulin light and heavy chain CDR sequences, one or more of which differs by no more than one amino acid from the CDR1 sequence of SEQ ID NO: 204, the CDR2 sequence of GAS, the CDR3 sequence of SEQ ID NO: 206, the CDR1 sequence of SEQ ID NO: 117, the CDR2 sequence of SEQ ID NO: 118, and the CDR3 sequence of SEQ ID NO: 119.

4. A composition comprising an antibody or binding fragment thereof comprising immunoglobulin light chain CDRs and/or immunoglobulin heavy chain CDRS comprising polypeptides of any one of embodiments 1-3.

5. A polypeptide comprising an amino acid sequence having at least 95% sequence identity to the immunoglobulin light chain set forth in SEQ ID NO: 4, wherein said polypeptide binds to the E protein of Zika virus.

6. A polypeptide comprising an amino acid sequence having at least 95% sequence identity to the immunoglobulin heavy chain set forth in SEQ ID NO: 2, wherein said polypeptide binds to the E protein of Zika virus.

7. One or more polypeptides comprising an amino acid sequence having at least 95% sequence identity to the immunoglobulin light chain set forth in SEQ ID NO: 4 and an amino acid sequence having at least 95% sequence identity to the immunoglobulin heavy chain set forth in SEQ ID NO: 2, wherein said one or more polypeptides binds to the E protein of Zika virus.

8. A composition comprising an antibody or binding fragment thereof comprising an immunoglobulin light chain and/or immunoglobulin heavy chain comprising the polypeptides of any one of embodiments 5-7.

9. A polypeptide that binds to the E protein of Zika virus and comprises three immunoglobulin light chain CDR sequences, one or more of which differs by no more than one amino acid from the CDR1 sequence of SEQ ID NO: 282, the CDR2 sequence of GAS, and the CDR3 sequence of SEQ ID NO: 284.

10. A polypeptide that binds to the E protein of Zika virus and comprises three immunoglobulin heavy chain CDR sequences, one or more of which differs by no more than one amino acid from the CDR1 sequence of SEQ ID NO: 195, the CDR2 sequence of SEQ ID NO: 196, and the CDR3 sequence of SEQ ID NO: 197.

11. One or more polypeptides that bind to the E protein of Zika virus and together comprise six immunoglobulin light and heavy chain CDR sequences, one or more of which differs by no more than one amino acid from the CDR1 sequence of SEQ ID NO: 282, the CDR2 sequence of AAS, the CDR3 sequence of SEQ ID NO: 284, the CDR1 sequence of SEQ ID NO: 195, the CDR2 sequence of SEQ ID NO: 196, and the CDR3 sequence of SEQ ID NO: 197 CDR3.

12. A composition comprising an antibody or binding fragment thereof comprising immunoglobulin light chain CDRs and/or immunoglobulin heavy chain CDRS encoded by the polypeptides of any one of embodiments 9-11.

13. A polypeptide comprising an amino acid sequence having at least 95% sequence identity to the immunoglobulin light chain set forth in SEQ ID NO: 8, wherein said polypeptide binds to the E protein of Zika virus.

14. A polypeptide comprising an amino acid sequence having at least 95% sequence identity to the immunoglobulin heavy chain set forth in SEQ ID NO: 6, wherein said polypeptide binds to the E protein of Zika virus.

15. One or more polypeptides comprising an amino acid sequence having at least 95% sequence identity to the immunoglobulin light chain set forth in SEQ ID NO: 8 and an amino acid sequence having at least 95% sequence identity to the immunoglobulin heavy chain set forth in SEQ ID NO: 6, wherein said one or more polypeptides binds to the E protein of Zika virus.

16. A composition comprising, an antibody or binding fragment thereof comprising an immunoglobulin light chain and/or immunoglobulin heavy chain encoded by the polypeptides of any one of embodiments 13-15.

17. A polypeptide that binds to the E protein of Zika virus and comprises three immunoglobulin light chain CDR sequences, one or more of which differs by no more than one amino acid from the CDR1 sequence of SEQ ID NO: 234, the CDR2 sequence of GAS, and the CDR3 sequence of SEQ ID NO: 236.

18. A polypeptide that binds to the E protein of Zika virus and comprises three immunoglobulin heavy chain CDR sequences, one or more of which differs by no more than one amino acid from the CDR1 sequence of SEQ ID NO: 147, the CDR2 sequence of SEQ ID NO: 148 CDR2, and the CDR3 sequence of SEQ ID NO: 149.

19. One or more polypeptides that bind to the E protein of Zika virus and together comprise six immunoglobulin light and heavy chain CDR sequences, one or more of which differs by no more than one amino acid from the CDR1 sequence of SEQ ID NO: 234, CDR2 sequence of GAS, CDR3 sequence of SEQ ID NO: 236, CDR1 sequence of SEQ ID NO: 147, CDR2 sequence of SEQ ID NO: 148, and CDR3 sequence of SEQ ID NO: 149.

20. A composition comprising an antibody or binding fragment thereof comprising immunoglobulin light chain CDRs and/or immunoglobulin heavy chain CDRS encoded by the polypeptides of any one of embodiments 17-19.

21. A polypeptide comprising an amino acid sequence having at least 95% sequence identity to the immunoglobulin light chain set forth in SEQ ID NO: 12, wherein said polypeptide binds to the E protein of Zika virus.

22. A polypeptide comprising an amino acid sequence having at least 95% sequence identity to the immunoglobulin heavy chain set forth in SEQ ID NO: 10, wherein said polypeptide binds to the E protein of Zika virus.

23. One or more polypeptides comprising an amino acid sequence having at least 95% sequence identity to the immunoglobulin light chain set forth in SEQ ID NO: 12 and an amino acid sequence having at least 95% sequence identity to the immunoglobulin heavy chain set forth in SEQ ID NO: 10, wherein said one or more polypeptides binds to the E protein of Zika virus.

24. A composition comprising an antibody or binding fragment thereof comprising an immunoglobulin light chain and/or immunoglobulin heavy chain encoded by the polypeptides of any one of embodiments 21-23.

25. A polypeptide that binds to the E protein of Zika virus and comprises three immunoglobulin light chain CDR sequences, one or more of which differs by no more than one amino acid from the CDR1 sequence of SEQ ID NO: 249, CDR2 sequence of EGT, and CDR3 sequence of SEQ ID NO: 251.

26. A polypeptide that binds to the E protein of Zika virus and comprises three immunoglobulin heavy chain CDR sequences, one or more of which differs by no more than one amino acid from the CDR1 sequence of SEQ ID NO: 162, CDR2 sequence of SEQ ID NO: 163, and CDR3 sequence of SEQ ID NO: 164.

27. One or more polypeptides that bind to the E protein of Zika virus and together comprise six immunoglobulin light and heavy chain CDR sequences, one or more of which differs by no more than one amino acid from the CDR1 sequence of SEQ ID NO: 249, CDR2 sequence of EGT, CDR3 sequence of SEQ ID NO: 251, CDR1 sequence of SEQ ID NO: 162, CDR2 sequence of SEQ ID NO: 163, and CDR3 sequence of SEQ ID NO: 164.

28. A composition comprising, an antibody or binding fragment thereof comprising immunoglobulin light chain CDRs and/or immunoglobulin heavy chain CDRS encoded by the polypeptides of any one of embodiments 25-27.

29. A polypeptide comprising an amino acid sequence having at least 95% sequence identity to the immunoglobulin light chain set forth in SEQ ID NO: 16, wherein said polypeptide binds to the E protein of Zika virus.

30. A polypeptide comprising an amino acid sequence having at least 95% sequence identity to the immunoglobulin heavy chain set forth in SEQ ID NO: 14, wherein said polypeptide binds to the E protein of Zika virus.

31. One or more polypeptides comprising an amino acid sequence having at least 95% sequence identity to the immunoglobulin light chain set forth in SEQ ID NO: 16 and an amino acid sequence having at least 95% sequence identity to the immunoglobulin heavy chain set forth in SEQ ID NO: 14, wherein said one or more polypeptides binds to the E protein of Zika virus.

32. A composition comprising, an antibody or binding fragment thereof comprising an immunoglobulin light chain and/or immunoglobulin heavy chain encoded by the polypeptides of any one of embodiments 29-31.

33. A polypeptide that binds to the E protein of Zika virus and comprises three immunoglobulin light chain CDR sequences, one or more of which differs by no more than one amino acid from the CDR1 sequence of SEQ ID NO: 207, CDR2 sequence of AAS, and CDR3 sequence of SEQ ID NO: 209.

34. A polypeptide that binds to the E protein of Zika virus and comprises three immunoglobulin heavy chain CDR sequences, one or more of which differs by no more than one amino acid from the CDR1 sequence of SEQ ID NO: 120, CDR2 sequence of SEQ ID NO: 121, and CDR3 sequence of SEQ ID NO: 122.

35. One or more polypeptides that bind to the E protein of Zika virus and together comprise six immunoglobulin light and heavy chain CDR sequences, one or more of which differs by no more than one amino acid from the CDR1 sequence of SEQ ID NO: 207, CDR2 sequence of AAS, CDR3 sequence of SEQ ID NO: 209, CDR1 sequence of SEQ ID NO: 120, CDR2 sequence of SEQ ID NO: 121, and CDR3 sequence of SEQ ID NO: 122.

36. A composition comprising an antibody or binding fragment thereof comprising immunoglobulin light chain CDRs and/or immunoglobulin heavy chain CDRS encoded by the polypeptides of any one of embodiments 33-35.

37. A polypeptide comprising an amino acid sequence having at least 95% sequence identity to the immunoglobulin heavy chain set forth in SEQ ID NO: 20, wherein said polypeptide binds to the E protein of Zika virus.

38. A polypeptide comprising an amino acid sequence having at least 95% sequence identity to the immunoglobulin heavy chain set forth in SEQ ID NO: 18, wherein said polypeptide binds to the E protein of Zika virus.

39. One or more polypeptides comprising an amino acid sequence having at least 95% sequence identity to the immunoglobulin light chain set forth in SEQ ID NO: 20 and an amino acid sequence having at least 95% sequence identity to the immunoglobulin heavy chain set forth in SEQ ID NO: 18, wherein said one or more polypeptides binds to the E protein of Zika virus.

40. A composition comprising an antibody or binding fragment thereof comprising an immunoglobulin light chain and/or immunoglobulin heavy chain encoded by the polypeptides of any one of embodiments 37-39.

41. A polypeptide that binds to the E protein of Zika virus and comprises three immunoglobulin light chain CDR sequences, one or more of which differs by no more than one amino acid from the CDR1 sequence of SEQ ID NO: 210, CDR2 sequence of AAS, and CDR3 sequence of SEQ ID NO: 212.

42. A polypeptide that binds to the E protein of Zika virus and comprises three immunoglobulin heavy chain CDR sequences, one or more of which differs by no more than one amino acid from the CDR1 sequence of SEQ ID NO: 123, CDR2 sequence of SEQ ID NO: 124, and CDR3 sequence of SEQ ID NO: 125.

43. One or more polypeptides that bind to the E protein of Zika virus and together comprise six immunoglobulin light and heavy chain CDR sequences, one or more of which differs by no more than one amino acid from the CDR1 sequence of SEQ ID NO: 210, CDR2 sequence of AAS, CDR3 sequence of SEQ ID NO: 212, CDR1 sequence of SEQ ID NO: 123, CDR2 sequence of SEQ ID NO: 124, and CDR3 sequence of SEQ ID NO: 125.

44. A composition comprising an antibody or binding fragment thereof comprising immunoglobulin light chain CDRs and/or immunoglobulin heavy chain CDRS encoded by the polypeptides of any one of embodiments 41-43.

45. A polypeptide comprising an amino acid sequence having at least 95% sequence identity to the immunoglobulin light chain set forth in SEQ ID NO: 24, wherein said polypeptide binds to the E protein of Zika virus.

46. A polypeptide comprising an amino acid sequence having at least 95% sequence identity to the immunoglobulin heavy chain set forth in SEQ ID NO: 22, wherein said polypeptide binds to the E protein of Zika virus.

47. One or more polypeptides comprising at least 95% sequence identity to the immunoglobulin light chain set forth in SEQ ID NO: 24 and an amino acid sequence having at least 95% sequence identity to the immunoglobulin heavy chain set forth in SEQ ID NO: 22, wherein said one or more polypeptides binds to the E protein of Zika virus.

48. A composition comprising an antibody or binding fragment thereof comprising an immunoglobulin light chain and/or immunoglobulin heavy chain encoded by the polypeptides of any one of embodiments 45-47.

49. A polypeptide that binds to the E protein of Zika virus and comprises three immunoglobulin light chain CDR sequences, one or more of which differs by no more than one amino acid from the CDR1 sequence of SEQ ID NO: 213, CDR2 sequence of GAS, and CDR3 sequence of SEQ ID NO: 215.

50. A polypeptide that binds to the E protein of Zika virus and comprises three immunoglobulin heavy chain CDR sequences, one or more of which differs by no more than one amino acid from the CDR1 sequence of SEQ ID NO: 126, CDR2 sequence of SEQ ID NO: 127, and CDR3 sequence of SEQ ID NO: 128.

51. One or more polypeptides that bind to the E protein of Zika virus and together comprise six immunoglobulin light and heavy chain CDR sequences, one or more of which differs by no more than one amino acid from the CDR1 sequence of SEQ ID NO: 213, CDR2 sequence of GAS, CDR3 sequence of SEQ ID NO: 215, CDR1 sequence of SEQ ID NO: 126, CDR2 sequence of SEQ ID NO: 127, and CDR3 sequence of SEQ ID NO: 128.

52. A composition comprising an antibody or binding fragment thereof comprising immunoglobulin light chain CDRs and/or immunoglobulin heavy chain CDRS encoded by the polypeptides of any one of embodiments 49-51.

53. A polypeptide comprising an amino acid sequence having at least 95% sequence identity to the immunoglobulin light chain set forth in SEQ ID NO: 28, wherein said polypeptide binds to the E protein of Zika virus.

54. A polypeptide comprising an amino acid sequence having at least 95% sequence identity to the immunoglobulin heavy chain set forth in SEQ ID NO: 26, wherein said polypeptide binds to the E protein of Zika virus.

55. One or more polypeptides comprising an amino acid sequence having at least 95% sequence identity to the immunoglobulin light chain set forth in SEQ ID NO: 28 and an amino acid sequence having at least 95% sequence identity to the immunoglobulin heavy chain set forth in SEQ ID NO: 26, wherein said one or more polypeptides binds to the E protein of Zika virus.

56. A composition comprising an antibody or binding fragment thereof comprising an immunoglobulin light chain and/or immunoglobulin heavy chain encoded by the polypeptides of any one of embodiments 53-55.

57. A polypeptide that binds to the E protein of Zika virus and comprises three immunoglobulin light chain CDR sequences, one or more of which differs by no more than one amino acid from the CDR1 sequence of SEQ ID NO: 216, CDR2 sequence of LGF, and CDR3 sequence of SEQ ID NO: 218.

58. A polypeptide that binds to the E protein of Zika virus and comprises three immunoglobulin heavy chain CDR sequences, one or more of which differs by no more than one amino acid from the CDR1 sequence of SEQ ID NO: 129, CDR2 sequence of SEQ ID NO: 130, and CDR3 sequence of SEQ ID NO: 131.

59. One or more polypeptides that bind to the E protein of Zika virus and together comprise six immunoglobulin light and heavy chain CDR sequences, one or more of which differs by no more than one amino acid from the CDR1 sequence of SEQ ID NO: 216, CDR2 sequence of LGF, CDR3 sequence of SEQ ID NO: 218, CDR1 sequence of SEQ ID NO: 129, CDR2 sequence of SEQ ID NO: 130, and CDR3 sequence of SEQ ID NO: 131.

60. A composition comprising an antibody or binding fragment thereof comprising immunoglobulin light chain CDRs and/or immunoglobulin heavy chain CDRS encoded by the polypeptides of any one of embodiments 57-59.

61. A polypeptide comprising an amino acid sequence having at least 95% sequence identity to the immunoglobulin light chain set forth in SEQ ID NO: 32, wherein said polypeptide binds to the E protein of Zika virus.

62. A polypeptide comprising an amino acid sequence having at least 95% sequence identity to the immunoglobulin heavy chain set forth in SEQ ID NO: 30, wherein said polypeptide binds to the E protein of Zika virus.

63. One or more polypeptides comprising an amino acid sequence having at least 95% sequence identity to the immunoglobulin light chain set forth in SEQ ID NO: 32 and an amino acid sequence having at least 95% sequence identity to the immunoglobulin heavy chain set forth in SEQ ID NO: 30, wherein said one or more polypeptides binds to the E protein of Zika virus.

64. A composition comprising an antibody or binding fragment thereof comprising an immunoglobulin light chain and/or immunoglobulin heavy chain encoded by the polypeptides of any one of embodiments 61-63.

65. A polypeptide that binds to the E protein of Zika virus and comprises three immunoglobulin light chain CDR sequences, one or more of which differs by no more than one amino acid from the CDR1 sequence of SEQ ID NO: 219, CDR2 sequence of AAS, and CDR3 sequence of SEQ ID NO: 221.

66. A polypeptide that binds to the E protein of Zika virus and comprises three immunoglobulin heavy chain CDR sequences, one or more of which differs by no more than one amino acid from the CDR1 sequence of SEQ ID NO: 132, CDR2 sequence of SEQ ID NO: 133, and CDR3 sequence of SEQ ID NO: 134.

67. One or more polypeptides that bind to the E protein of Zika virus and together comprise six immunoglobulin light and heavy chain CDR sequences, one or more of which differs by no more than one amino acid from the CDR1 sequence of SEQ ID NO: 219, CDR2 sequence of AAS, CDR3 sequence of SEQ ID NO: 221, CDR1 sequence of SEQ ID NO: 132, CDR2 sequence of SEQ ID NO: 133, and CDR3 sequence of SEQ ID NO: 134.

68. A composition comprising an antibody or binding fragment thereof comprising immunoglobulin light chain CDRs and/or immunoglobulin heavy chain CDRS encoded by the polypeptides of any one of embodiments 65-67.

69. A polypeptide comprising an amino acid sequence having at least 95% sequence identity to the immunoglobulin light chain set forth in SEQ ID NO: 36, wherein said polypeptide binds to the E protein of Zika virus.

70. A polypeptide comprising an amino acid sequence having at least 95% sequence identity to the immunoglobulin heavy chain set forth in SEQ ID NO: 34, wherein said polypeptide binds to the E protein of Zika virus.

71. One or more polypeptides comprising an amino acid sequence having at least 95% sequence identity to the immunoglobulin light chain set forth in SEQ ID NO: 36 and an amino acid sequence having at least 95% sequence identity to the immunoglobulin heavy chain set forth in SEQ ID NO: 34, wherein said one or more, polypeptides binds to the E protein of Zika virus.

72. A composition comprising an antibody or binding fragment thereof comprising an immunoglobulin light chain and/or immunoglobulin heavy chain encoded by the polypeptides of any one of embodiments 69-71.

73. A polypeptide that binds to the E protein of Zika virus and comprises three immunoglobulin light chain CDR sequences, one or more of which differs by no more than one amino acid from the CDR1 sequence of SEQ ID NO: 222, CDR2 sequence of YAS, and CDR3 sequence of SEQ ID NO: 224.

74. A polypeptide that binds to the E protein of Zika virus and comprises three immunoglobulin heavy chain CDR sequences, one or more of which differs by no more than one amino acid from the CDR1 sequence of SEQ ID NO: 135, CDR2 sequence of SEQ ID NO: 136, and CDR3 sequence of SEQ ID NO: 137.

75. One or more polypeptides that bind to the E protein of Zika virus and together comprise six immunoglobulin light and heavy chain CDR sequences, one or more of which differs by no more than one amino acid from the CDR1 sequence of SEQ ID NO: 222, CDR2 sequence of YAS, CDR3 sequence of SEQ ID NO: 224, CDR1 sequence of SEQ ID NO: 135, CDR2 sequence of SEQ ID NO: 136, and CDR3 sequence of SEQ ID NO: 137.

76. A composition comprising an antibody or binding fragment thereof comprising immunoglobulin light chain CDRs and/or immunoglobulin heavy chain CDRS encoded by the polypeptides of any one of embodiments 73-75.

77. A polypeptide comprising an amino acid sequence having at least 95% sequence identity to the immunoglobulin light chain set forth in SEQ ID NO: 40, wherein said polypeptide binds to the E protein of Zika virus.

78. A polypeptide comprising an amino acid sequence having at least 95% sequence identity to the immunoglobulin heavy chain set forth in SEQ ID NO: 38, wherein said polypeptide binds to the E protein of Zika virus.

79. One or more polypeptides comprising an amino acid sequence having at least 95% sequence identity to the immunoglobulin light chain set forth in SEQ ID NO: 40 and an amino acid sequence having at least 95% sequence identity to the immunoglobulin heavy chain set forth in SEQ ID NO: 38, wherein said one or more polypeptides binds to the E protein of Zika virus.

80. A composition comprising an antibody or binding fragment thereof comprising an immunoglobulin light chain and/or immunoglobulin heavy chain encoded by the polypeptides of any one of embodiments 77-79.

81. A polypeptide that binds to the E protein of Zika virus and comprises three immunoglobulin light chain CDR sequences, one or more of which differs by no more than one amino acid from the CDR1 sequence of SEQ ID NO: 225, CDR2 sequence of KVS, and CDR3 sequence of SEQ ID NO: 227.

82. A polypeptide that binds to the E protein of Zika virus and comprises three immunoglobulin heavy chain CDR sequences, one or more of which differs by no more than one amino acid from the CDR1 sequence of SEQ ID NO: 138, CDR2 sequence of SEQ ID NO: 139, and CDR3 sequence of SEQ ID NO: 140.

83. One or more polypeptides that bind to the E protein of Zika virus and together comprise six immunoglobulin light and heavy chain CDR sequences, one or more of which differs by no more than one amino acid from the CDR1 sequence of SEQ ID NO: 225, CDR2 sequence of KVS, CDR3 sequence of SEQ ID NO: 227, CDR1 sequence of SEQ ID NO: 138, CDR2 sequence of SEQ ID NO: 139, and CDR3 sequence of SEQ ID NO: 140.

84. A composition comprising an antibody or binding fragment thereof comprising immunoglobulin light chain CDRs and/or immunoglobulin heavy chain CDRS encoded by the polypeptides of any one of embodiments 81-83.

85. A polypeptide comprising an amino acid sequence having at least 95% sequence identity to the immunoglobulin light chain set forth in SEQ ID NO: 44, wherein said polypeptide binds to the E protein of Zika virus.

86. A polypeptide comprising an amino acid sequence having at least 95% sequence identity to the immunoglobulin heavy chain set forth in SEQ ID NO: 42, wherein said one or more polypeptides binds to the E protein of Zika virus.

87. One or more polypeptides comprising an amino acid sequence having at least 95% sequence identity to the immunoglobulin light chain set forth in SEQ ID NO: 44 and an amino acid sequence having at least 95% sequence identity to the immunoglobulin heavy chain set forth in SEQ ID NO: 42.

88. A composition comprising an antibody or binding fragment thereof comprising an immunoglobulin light chain and/or immunoglobulin heavy chain encoded by the polypeptides of any one of embodiments 85-87.

89. A polypeptide that binds to the E protein of Zika virus and comprises three immunoglobulin light chain CDR sequences, one or more of which differs by no more than one amino acid from the CDR1 sequence of SEQ ID NO: 228, CDR2 sequence of DVS, and CDR3 sequence of SEQ ID NO: 230.

90. A polypeptide that binds to the E protein of Zika virus and comprises three immunoglobulin heavy chain CDR sequences, one or more of which differs by no more than one amino acid from the CDR1 sequence of SEQ ID NO: 141, CDR2 sequence of SEQ ID NO: 142, and CDR3 sequence of SEQ ID NO: 143.

91. One or more polypeptides that bind to the E protein of Zika virus and together comprise six immunoglobulin light and heavy chain CDR sequences, one or more of which differs by no more than one amino acid from the CDR1 sequence of SEQ ID NO: 228, CDR2 sequence of DVS, CDR3 sequence of SEQ ID NO: 230, CDR1 sequence of SEQ ID NO: 141, CDR2 sequence of SEQ ID NO: 142, and CDR3 sequence of SEQ ID NO: 143.

92. A composition comprising an antibody or binding fragment thereof comprising immunoglobulin light chain CDRs and/or immunoglobulin heavy chain CDRS encoded by the polypeptides of any one of embodiments 89-91.

93. A polypeptide comprising an amino acid sequence having at least 95% sequence identity to the immunoglobulin light chain set forth in SEQ ID NO: 48, wherein said polypeptide binds to the E protein of Zika virus.

94. A polypeptide comprising an amino acid sequence having at least 95% sequence identity to the immunoglobulin heavy chain set forth in SEQ ID NO: 46, wherein said polypeptide binds to the E protein of Zika virus.

95. One or more polypeptides comprising an amino acid sequence having at least 95% sequence identity to the immunoglobulin light chain set forth in SEQ ID NO: 48 and an amino acid sequence having at least 95% sequence identity to the immunoglobulin heavy chain set forth in SEQ ID NO: 46, wherein said one or more polypeptides binds to the E protein of Zika virus.

96. A composition comprising an antibody or binding fragment thereof comprising an immunoglobulin light chain and/or immunoglobulin heavy chain encoded by the polypeptides of any one of embodiments 93-95.

97. A polypeptide that binds to the E protein of Zika virus and comprises three immunoglobulin light chain CDR sequences, one or more of which differs by no more than one amino acid from the CDR1 sequence of SEQ ID NO: 231, CDR2 sequence of EDS, and CDR3 sequence of SEQ ID NO: 233.

98. A polypeptide that binds to the E protein of Zika virus and comprises three immunoglobulin heavy chain CDR sequences, one or more of which differs by no more than one amino acid from the CDR1 sequence of SEQ ID NO: 145, CDR2 sequence of SEQ ID NO: 146, and CDR3 sequence of SEQ ID NO: 147.

99. One or more polypeptides that bind to the E protein of Zika virus and together comprise six immunoglobulin light and heavy chain CDR sequences, one or more of which differs by no more than one amino acid from the CDR1 sequence of SEQ ID NO: 231, CDR2 sequence of EDS, CDR3 sequence of SEQ ID NO: 233, CDR1 sequence of SEQ ID NO: 145, CDR2 sequence of SEQ ID NO: 146, and CDR3 sequence of SEQ ID NO: 147.

100. A composition comprising an antibody or binding fragment thereof comprising immunoglobulin light chain CDRs and/or immunoglobulin heavy chain CDRS encoded by the polypeptides of any one of embodiments 97-99.

101. A polypeptide comprising an amino acid sequence having at least 95% sequence identity to the immunoglobulin light chain set forth in SEQ ID NO: 52, wherein said polypeptide binds to the E protein of Zika virus.

102. A polypeptide comprising an amino acid sequence having at least 95% sequence identity to the immunoglobulin heavy chain set forth in SEQ ID NO: 50, wherein said polypeptide binds to the E protein of Zika virus.

103. One or more polypeptides comprising an amino acid sequence having at least 95% sequence identity to the immunoglobulin light chain set forth in SEQ ID NO: 52 and an amino acid sequence having at least 95% sequence identity to the immunoglobulin heavy chain set forth in SEQ ID NO: 50, wherein said one or more polypeptides binds to the E protein of Zika virus.

104. A composition comprising an antibody or binding fragment thereof comprising an immunoglobulin light chain and/or immunoglobulin heavy chain encoded by the polypeptides of any one of embodiments 101-103.

105. A polypeptide that binds to the E protein of Zika virus and comprises three immunoglobulin light chain CDR sequences, one or more of which differs by no more than one amino acid from the CDR1 sequence of SEQ ID NO: 237, CDR2 sequence of EDS, and CDR3 sequence of SEQ ID NO: 239.

106. A polypeptide that binds to the E protein of Zika virus and comprises three immunoglobulin heavy chain CDR sequences, one or more of which differs by no more than one amino acid from the CDR1 sequence of SEQ ID NO: 150, CDR2 sequence of SEQ ID NO: 151, and CDR3 sequence of SEQ ID NO: 152.

107. One or more polypeptides that bind to the E protein of Zika virus and together comprise six immunoglobulin light and heavy chain CDR sequences, one or more of which differs by no more than one amino acid from the CDR1 sequence of SEQ ID NO: 237, CDR2 sequence of EDS, CDR3 sequence of SEQ ID NO: 239, CDR1 sequence of SEQ ID NO: 150, CDR2 sequence of SEQ ID NO: 151, and CDR3 sequence of SEQ ID NO: 152.

108. A composition comprising an antibody or binding fragment thereof comprising immunoglobulin light chain CDRs and/or immunoglobulin heavy chain CDRS encoded by the polypeptides of any one of embodiments 105-107.

109. A polypeptide comprising an amino acid sequence having at least 95% sequence identity to the immunoglobulin light chain set forth in SEQ ID NO: 56, wherein said polypeptide binds to the E protein of Zika virus.

110. A polypeptide comprising an amino acid sequence having at least 95% sequence identity to the immunoglobulin heavy chain set forth in SEQ ID NO: 54, wherein said polypeptide binds to the E protein of Zika virus.

111. One or more polypeptides comprising an amino acid sequence having at least 95% sequence identity to the immunoglobulin light chain set forth in SEQ ID NO: 56 and an amino acid sequence having at least 95% sequence identity to the immunoglobulin heavy chain set forth in SEQ ID NO: 54, wherein said one or more polypeptides binds to the E protein of Zika virus.

112. A composition comprising an antibody or binding fragment thereof comprising an immunoglobulin light chain and/or immunoglobulin heavy chain encoded by the polypeptides of any one of embodiments 109-111.

113. A polypeptide that binds to the E protein of Zika virus and comprises three immunoglobulin light chain CDR sequences, one or more of which differs by no more than one amino acid from the CDR1 sequence of SEQ ID NO: 240, CDR2 sequence of GKN, and CDR3 sequence of SEQ ID NO: 242.

114. A polypeptide that binds to the E protein of Zika virus and comprises three immunoglobulin heavy chain CDR sequences, one or more of which differs by no more than one amino acid from the CDR1 sequence of SEQ ID NO: 153, CDR2 sequence of SEQ ID NO: 154, and CDR3 sequence of SEQ ID NO: 155.

115. One or more polypeptides that bind to the E protein of Zika virus and together comprise six immunoglobulin light and heavy chain CDR sequences, one or more of which differs by no more than one amino acid from the CDR1 sequence of SEQ ID NO: 240, CDR2 sequence of GKN, CDR3 sequence of SEQ ID NO: 242, CDR1 sequence of SEQ ID NO: 153, CDR2 sequence of SEQ ID NO: 154, and CDR3 sequence of SEQ ID NO: 155.

116. A composition comprising an antibody or binding fragment thereof comprising immunoglobulin light chain CDRs and/or immunoglobulin heavy chain CDRS encoded by the polypeptides of any one of embodiments 113-115.

117. A polypeptide comprising an amino acid sequence having at least 95% sequence identity to the immunoglobulin light chain set forth in SEQ ID NO: 60, wherein said polypeptide binds to the E protein of Zika virus.

118. A polypeptide comprising an amino acid sequence having at least 95% sequence identity to the immunoglobulin heavy chain set forth in SEQ ID NO: 58, wherein said polypeptide binds to the E protein of Zika virus.

119. One or more polypeptides comprising an amino acid sequence having at least 95% sequence identity to the immunoglobulin light chain set forth in SEQ ID NO: 60 and an amino acid sequence having at least 95% sequence identity to the immunoglobulin heavy chain set forth in SEQ ID NO: 58, wherein said one or more polypeptides binds to the E protein of Zika virus.

120. A composition comprising an antibody or binding fragment thereof comprising an immunoglobulin light chain and/or immunoglobulin heavy chain encoded by the polypeptides of any one of embodiments 117-119.

121. A polypeptide that binds to the E protein of Zika virus and comprises three immunoglobulin light chain CDR sequences, one or more of which differs by no more than one amino acid from the CDR1 sequence of SEQ ID NO: 243, CDR2 sequence of KAS, and CDR3 sequence of SEQ ID NO: 245.

122. A polypeptide that binds to the E protein of Zika virus and comprises three immunoglobulin heavy chain CDR sequences, one or more of which differs by no more than one amino acid from the CDR1 sequence of SEQ ID NO: 156, CDR2 sequence of SEQ ID NO: 157, and CDR3 sequence of SEQ ID NO: 158.

123. One or more polypeptides that bind to the E protein of Zika virus and together comprise six immunoglobulin light and heavy chain CDR sequences, one or more of which differs by no more than one amino acid from the CDR1 sequence of SEQ ID NO: 243, CDR2 sequence of KAS, CDR3 sequence of SEQ ID NO: 245, CDR1 sequence of SEQ ID NO: 156, CDR2 sequence of SEQ ID NO: 157, and CDR3 sequence of SEQ ID NO: 158.

124. A composition comprising an antibody or binding fragment thereof comprising immunoglobulin light chain CDRs and/or immunoglobulin heavy chain CDRS encoded by the polypeptides of any one of embodiments 121-123.

125. A polypeptide comprising an amino acid sequence having at least 95% sequence identity to the immunoglobulin light chain set forth in SEQ ID NO: 64, wherein said polypeptide binds to the E protein of Zika virus.

126. A polypeptide comprising an amino acid sequence having at least 95% sequence identity to the immunoglobulin heavy chain set forth in SEQ ID NO: 62, wherein said polypeptide binds to the E protein of Zika virus.

127. One or more polypeptides comprising an amino acid sequence having at least 95% sequence identity to the immunoglobulin light chain set forth in SEQ ID NO: 64 and an amino acid sequence having at least 95% sequence identity to the immunoglobulin heavy chain set forth in SEQ ID NO: 62, wherein said one or more polypeptides binds to the E protein of Zika virus.

128. A composition comprising an antibody or binding fragment thereof comprising an immunoglobulin light chain and/or immunoglobulin heavy chain encoded by the polypeptides of any one of embodiments 125-127.

129. A polypeptide that binds to the E protein of Zika virus and comprises three immunoglobulin light chain CDR sequences, one or more of which differs by no more than one amino acid from the CDR1 sequence of SEQ ID NO: 246, CDR2 sequence of KVS, and CDR3 sequence of SEQ ID NO: 248.

130. A polypeptide that binds to the E protein of Zika virus and comprises three immunoglobulin heavy chain CDR sequences, one or more of which differs by no more than one amino acid from the CDR1 sequence of SEQ ID NO: 159, CDR2 sequence of SEQ ID NO: 160, and CDR3 sequence of SEQ ID NO: 161.

131. One or more polypeptides that bind to the E protein of Zika virus and together comprise six immunoglobulin light and heavy chain CDR sequences, one or more of which differs by no more than one amino acid from the CDR1 sequence of SEQ ID NO: 246, CDR2 sequence of KVS, CDR3 sequence of SEQ ID NO: 248, CDR1 sequence of SEQ ID NO: 159, CDR2 sequence of SEQ ID NO: 160, and CDR3 sequence of SEQ ID NO: 161.

132. A composition comprising an antibody or binding fragment thereof comprising immunoglobulin light chain CDRs and/or immunoglobulin heavy chain CDRS encoded by the polypeptides of any one of embodiments 273-275.

133. A polypeptide comprising an amino acid sequence having at least 95% sequence identity to the immunoglobulin light chain set forth in SEQ ID NO: 68, wherein said polypeptide binds to the E protein of Zika virus.

134. A polypeptide comprising an amino acid sequence having at least 95% sequence identity to the immunoglobulin heavy chain set forth in SEQ ID NO: 66, wherein said polypeptide binds to the E protein of Zika virus.

135. One or more polypeptides comprising an amino acid sequence having at least 95% sequence identity to the immunoglobulin light chain set forth in SEQ ID NO: 68 and an amino acid sequence having at least 95% sequence identity to the immunoglobulin heavy chain set forth in SEQ ID NO: 66, wherein said one or more polypeptides binds to the E protein of Zika virus.

136. A composition comprising an antibody or binding fragment thereof comprising an immunoglobulin light chain and/or immunoglobulin heavy chain encoded by the polypeptides of any one of embodiments 277-279.

137. A polypeptide that binds to the E protein of Zika virus and comprises three immunoglobulin light chain CDR sequences, one or more of which differs by no more than one amino acid from the CDR1 sequence of SEQ ID NO: 252, CDR2 sequence of AAS, and CDR3 sequence of SEQ ID NO: 254.

138. A polypeptide that binds to the E protein of Zika virus and comprises three immunoglobulin heavy chain CDR sequences, one or more of which differs by no more than one amino acid from the CDR1 sequence of SEQ ID NO: 165, CDR2 sequence of SEQ ID NO: 166, and CDR3 sequence of SEQ ID NO: 167.

139. One or more polypeptides that bind to the E protein of Zika virus and together comprise six immunoglobulin light and heavy chain CDR sequences, one or more of which differs by no more than one amino acid from the CDR1 sequence of SEQ ID NO: 252, CDR2 sequence of AAS, CDR3 sequence of SEQ ID NO: 254, CDR1 sequence of SEQ ID NO: 165, CDR2 sequence of SEQ ID NO: 166, and CDR3 sequence of SEQ ID NO: 167.

140. A composition comprising an antibody or binding fragment thereof comprising immunoglobulin light chain CDRs and/or immunoglobulin heavy chain CDRS encoded by the polypeptides of any one of embodiments 137-139.

141. A polypeptide comprising an amino acid sequence having at least 95% sequence identity to the immunoglobulin light chain set forth in SEQ ID NO: 72, wherein said polypeptide binds to the E protein of Zika virus.

142. A polypeptide comprising an amino acid sequence having at least 95% sequence identity to the immunoglobulin heavy chain set forth in SEQ ID NO: 70, wherein said polypeptide binds to the E protein of Zika virus.

143. One or more polypeptides comprising an amino acid sequence having at least 95% sequence identity to the immunoglobulin light chain set forth in SEQ ID NO: 72 and an amino acid sequence having at least 95% sequence identity to the immunoglobulin heavy chain set forth in SEQ ID NO: 70, wherein said one or more polypeptides binds to the E protein of Zika virus.

144. A composition comprising an antibody or binding fragment thereof comprising an immunoglobulin light chain and/or immunoglobulin heavy chain encoded by the polypeptides of any one of embodiments 141-143.

145. A polypeptide that binds to the E protein of Zika virus and comprises three immunoglobulin light chain CDR sequences, one or more of which differs by no more than one amino acid from the CDR1 sequence of SEQ ID NO: 255, CDR2 sequence of QDS, and CDR3 sequence of SEQ ID NO: 257.

146. A polypeptide that binds to the E protein of Zika virus and comprises three immunoglobulin heavy chain CDR sequences, one or more of which differs by no more than one amino acid from the CDR1 sequence of SEQ ID NO: 168, CDR2 sequence of SEQ ID NO: 169, and CDR3 sequence of SEQ ID NO: 170.

147. One or more polypeptides that bind to the E protein of Zika virus and together comprise six immunoglobulin light and heavy chain CDR sequences, one or more of which differs by no more than one amino acid from the CDR1 sequence of SEQ ID NO: 255, CDR2 sequence of QDS, CDR3 sequence of SEQ ID NO: 257, CDR1 sequence of SEQ ID NO: 168, CDR2 sequence of SEQ ID NO: 169, and CDR3 sequence of SEQ ID NO: 170.

148. A composition comprising an antibody or binding fragment thereof comprising immunoglobulin light chain CDRs and/or immunoglobulin heavy chain CDRS encoded by the polypeptides of any one of embodiments 146-147.

149. A polypeptide comprising an amino acid sequence having at least 95% sequence identity to the immunoglobulin light chain set forth in SEQ ID NO: 76, wherein said polypeptide binds to the E protein of Zika virus.

150. A polypeptide comprising an amino acid sequence having at least 95% sequence identity to the immunoglobulin heavy chain set forth in SEQ ID NO: 74, wherein said polypeptide binds to the E protein of Zika virus.

151. One or more polypeptides comprising an amino acid sequence having at least 95% sequence identity the immunoglobulin light chain set forth in SEQ ID NO: 76 and an amino acid sequence having at least 95% sequence identity to the immunoglobulin heavy chain set forth in SEQ ID NO: 74, wherein said one or more polypeptides binds to the E protein of Zika virus.

152. A composition comprising an antibody or binding fragment thereof comprising an immunoglobulin light chain and/or immunoglobulin heavy chain encoded by the polypeptides of any one of embodiments 149-151.

153. A polypeptide that binds to the E protein of Zika virus and comprises three immunoglobulin light chain CDR sequences, one or more of which differs by no more than one amino acid from the CDR1 sequence of SEQ ID NO: 258, CDR2 sequence of QDS, and CDR3 sequence of SEQ ID NO: 260.

154. A polypeptide that binds to the E protein of Zika virus and comprises three immunoglobulin heavy chain CDR sequences, one or more of which differs by no more than one amino acid from the CDR1 sequence of SEQ ID NO: 171, CDR2 sequence of SEQ ID NO: 172, and CDR3 sequence of SEQ ID NO: 173.

155. One or more polypeptides that bind to the E protein of Zika virus and together comprise six immunoglobulin light and heavy chain CDR sequences, one or more of which differs by no more than one amino acid from the CDR1 sequence of SEQ ID NO: 258, CDR2 sequence of QDS, CDR3 sequence of SEQ ID NO: 260, CDR1 sequence of SEQ ID NO: 171, CDR2 sequence of SEQ ID NO: 172, and CDR3 sequence of SEQ ID NO: 173.

156. A composition comprising an antibody or binding fragment thereof comprising immunoglobulin light chain CDRs and/or immunoglobulin heavy chain CDRS encoded by the polypeptides of any one of embodiments 156-160.

157. A polypeptide comprising an amino acid sequence having at least 95% sequence identity to the immunoglobulin light chain set forth in SEQ ID NO: 80, wherein said polypeptide binds to the E protein of Zika virus.

158. A polypeptide comprising an amino acid sequence having at least 95% sequence identity to the immunoglobulin heavy chain set forth in SEQ ID NO: 78, wherein said polypeptide binds to the E protein of Zika virus.

159. One or more polypeptides comprising an amino acid sequence having at least 95% sequence identity to the immunoglobulin light chain set forth in SEQ ID NO: 80 and at least 95% sequence identity to the immunoglobulin heavy chain set forth in SEQ ID NO: 78, wherein said one or more polypeptides binds to the E protein of Zika virus.

160. A composition comprising an antibody or binding fragment thereof comprising an immunoglobulin light chain and/or immunoglobulin heavy chain encoded by the polypeptides of any one of embodiments 157-159.

161. A polypeptide that binds to the E protein of Zika virus and comprises three immunoglobulin light chain CDR sequences, one or more of which differs by no more than one amino acid from the CDR1 sequence of SEQ ID NO: 261, CDR2 sequence of GAS, and CDR3 sequence of SEQ ID NO: 263.

162. A polypeptide that binds to the E protein of Zika virus and comprises three immunoglobulin heavy chain CDR sequences, one or more of which differs by no more than one amino acid from the CDR1 sequence of SEQ ID NO: 174, CDR2 sequence of SEQ ID NO: 175, and CDR3 sequence of SEQ ID NO: 176.

163. One or more polypeptides that bind to the E protein of Zika virus and together comprise six immunoglobulin light and heavy chain CDR sequences, one or more of which differs by no more than one amino acid from the CDR1 sequence of SEQ ID NO: 261, CDR2 sequence of GAS, CDR3 sequence of SEQ ID NO: 263, CDR1 sequence of SEQ ID NO: 174, CDR2 sequence of SEQ ID NO: 175, and CDR3 sequence of SEQ ID NO: 176.

164. A composition comprising an antibody or binding fragment thereof comprising immunoglobulin light chain CDRs and/or immunoglobulin heavy chain CDRS encoded by the polypeptides of any one of embodiments 161-163.

165. A polypeptide comprising an amino acid sequence having at least 95% sequence identity to the immunoglobulin light chain set forth in SEQ ID NO: 84, wherein said polypeptide binds to the E protein of Zika virus.

166. A polypeptide comprising an amino acid sequence having at least 95% sequence identity to the immunoglobulin heavy chain set forth in SEQ ID NO: 82, wherein said polypeptide binds to the E protein of Zika virus.

167. One or more polypeptides comprising an amino acid sequence having at least 95% sequence identity to the immunoglobulin light chain set forth in SEQ ID NO: 84 and an amino acid sequence having at least 95% sequence identity to the immunoglobulin heavy chain set forth in SEQ ID NO: 82, wherein said one or more polypeptides binds to the E protein of Zika virus.

168. A composition comprising an antibody or binding fragment thereof comprising an immunoglobulin light chain and/or immunoglobulin heavy chain encoded by the polypeptides of any one of embodiments 165-167.

169. A polypeptide that binds to the E protein of Zika virus and comprises three immunoglobulin light chain CDR sequences, one or more of which differs by no more than one amino acid from the CDR1 sequence of SEQ ID NO: 264, CDR2 sequence of EVS, and CDR3 sequence of SEQ ID NO: 266.

170. A polypeptide that binds to the E protein of Zika virus and comprises three immunoglobulin heavy chain CDR sequences, one or more of which differs by no more than one amino acid from the CDR1 sequence of SEQ ID NO: 177, CDR2 sequence of SEQ ID NO: 178, and CDR3 sequence of SEQ ID NO: 179.

171. One or more polypeptides that bind to the E protein of Zika virus and together comprise six immunoglobulin light and heavy chain CDR sequences, one or more of which differs by no more than one amino acid from the CDR1 sequence of SEQ ID NO: 264, CDR2 sequence of EVS, CDR3 sequence of SEQ ID NO: 266, CDR1 sequence of SEQ ID NO: 177, CDR2 sequence of SEQ ID NO: 178, and CDR3 sequence of SEQ ID NO: 179.

172. A composition comprising an antibody or binding fragment thereof comprising immunoglobulin light chain CDRs and/or immunoglobulin heavy chain CDRS encoded by the polypeptides of any one of embodiments 169-171.

173. A polypeptide comprising an amino acid sequence having at least 95% sequence identity to the immunoglobulin light chain set forth in SEQ ID NO: 88, wherein said polypeptide binds to the E protein of Zika virus.

174. A polypeptide comprising an amino acid sequence having at least 95% sequence identity to the immunoglobulin heavy chain set forth in SEQ ID NO: 86, wherein said polypeptide binds to the E protein of Zika virus.

175. One or more polypeptides comprising an amino acid sequence having at least 95% sequence identity to the immunoglobulin light chain set forth in SEQ ID NO: 88 and an amino acid sequence having at least 95% sequence identity to the immunoglobulin heavy chain set forth in SEQ ID NO: 86, wherein said one or more polypeptides binds to the E protein of Zika virus.

176. A composition comprising an antibody or binding fragment thereof comprising an immunoglobulin light chain and/or immunoglobulin heavy chain encoded by the polypeptides of any one of embodiments 173-175.

177. A polypeptide that binds to the E protein of Zika virus and comprises one immunoglobulin light chain CDR sequence, which differs by no more than one amino acid from the CDR3 sequence of SEQ ID NO: 269.

178. A polypeptide that binds to the E protein of Zika virus and comprises three immunoglobulin heavy chain CDR sequences, one or more of which differs by no more than one amino acid from the CDR1 sequence of SEQ ID NO: 180, CDR2 sequence of SEQ ID NO: 181, and CDR3 sequence of SEQ ID NO: 182.

179. One or more polypeptides that bind to the E protein of Zika virus and together comprise four immunoglobulin light and heavy chain CDR sequences, one or more of which differs by no more than one amino acid from the CDR3 sequence of SEQ ID NO: 269, CDR1 sequence of SEQ ID NO: 180, CDR2 sequence of SEQ ID NO: 181, and CDR3 sequence of SEQ ID NO: 182.

180. A composition comprising an antibody or binding fragment thereof comprising immunoglobulin light chain CDRs and/or immunoglobulin heavy chain CDRS encoded by the polypeptides of any one of embodiments 177-179.

181. A polypeptide comprising an amino acid sequence having at least 95% sequence identity to the immunoglobulin light chain set forth in SEQ ID NO: 92, wherein said polypeptide binds to the E protein of Zika virus.

182. A polypeptide comprising an amino acid sequence having at least 95% sequence identity to the immunoglobulin heavy chain set forth in SEQ ID NO: 90, wherein said polypeptide binds to the E protein of Zika virus.

183. One or more polypeptides comprising an amino acid sequence having at least 95% sequence identity to the immunoglobulin light chain set forth in SEQ ID NO: 92 and an amino acid sequence having at least 95% sequence identity to the immunoglobulin heavy chain set forth in SEQ ID NO: 90, wherein said one or more polypeptides binds to the E protein of Zika virus.

184. A composition comprising an antibody or binding fragment thereof comprising an immunoglobulin light chain and/or immunoglobulin heavy chain encoded by the polypeptides of any one of embodiments 181-183.

185. A polypeptide that binds to the E protein of Zika virus and comprises three immunoglobulin light chain CDR sequences, one or more of which differs by no more than one amino acid from the CDR1 sequence of SEQ ID NO: 270, CDR2 sequence of GAS, and CDR3 sequence of SEQ ID NO: 272.

186. A polypeptide that binds to the E protein of Zika virus and comprises three immunoglobulin heavy chain CDR sequences, one or more of which differs by no more than one amino acid from the CDR1 sequence of SEQ ID NO: 183, CDR2 sequence of SEQ ID NO: 184, and CDR3 sequence of SEQ ID NO: 185.

187. One or more polypeptides that bind to the E protein of Zika virus and together comprise six immunoglobulin light and heavy chain CDR sequences, one or more of which differs by no more than one amino acid from the CDR1 sequence of SEQ ID NO: 270, CDR2 sequence of GAS, and CDR3 sequence of SEQ ID NO: 272, CDR1 sequence of SEQ ID NO: 183, CDR2 sequence of SEQ ID NO: 184, and CDR3 sequence of SEQ ID NO: 185.

188. A composition comprising an antibody or binding fragment thereof comprising immunoglobulin light chain CDRs and/or immunoglobulin heavy chain CDRS encoded by the polypeptides of any one of embodiments 185-187.

189. A polypeptide comprising an amino acid sequence having at least 95% sequence identity to the immunoglobulin light chain set forth in SEQ ID NO: 96, wherein said polypeptide binds to the E protein of Zika virus.

190. A polypeptide comprising an amino acid sequence having at least 95% sequence identity to the immunoglobulin heavy chain set forth in SEQ ID NO: 94, wherein said polypeptide binds to the E protein of Zika virus.

191. One or more polypeptides comprising an amino acid sequence having at least 95% sequence identity to the immunoglobulin light chain set forth in SEQ ID NO: 96 and an amino acid sequence having at least 95% sequence identity to the immunoglobulin heavy chain set forth in SEQ ID NO: 94, wherein said one or more polypeptides binds to the E protein of Zika virus.

192. A composition comprising an antibody or binding fragment thereof comprising an immunoglobulin light chain and/or immunoglobulin heavy chain encoded by the polypeptides of any one of embodiments 189-191.

193. A polypeptide that binds to the E protein of Zika virus and comprises three immunoglobulin light chain CDR sequences, one or more of which differs by no more than one amino acid from the CDR1 sequence of SEQ ID NO: 273, CDR2 sequence of EVS, and CDR3 sequence of SEQ ID NO: 275.

194. A polypeptide that binds to the E protein of Zika virus and comprises three immunoglobulin heavy chain CDR sequences, one or more of which differs by no more than one amino acid from the CDR1 sequence of SEQ ID NO: 186, CDR2 sequence of SEQ ID NO: 187, and CDR3 sequence of SEQ ID NO: 188.

195 One or more polypeptides that bind to the E protein of Zika virus and together comprise six immunoglobulin light and heavy chain CDR sequences, one or more of which differs by no more than one amino acid from the CDR1 sequence of SEQ ID NO: 273, CDR2 sequence of EVS, CDR3 sequence of SEQ ID NO: 275, CDR1 sequence of SEQ ID NO: 186, CDR2 sequence of SEQ ID NO: 187, and CDR3 sequence of SEQ ID NO: 188.

196. A composition comprising an antibody or binding fragment thereof comprising immunoglobulin light chain CDRs and/or immunoglobulin heavy chain CDRS encoded by the polypeptides of any one of embodiments 193-195.

197. A polypeptide comprising an amino acid sequence having at least 95% sequence identity to the immunoglobulin light chain set forth in SEQ ID NO: 100, wherein said polypeptide binds to the E protein of Zika virus.

198. A polypeptide comprising an amino acid sequence having at least 95% sequence identity to the immunoglobulin heavy chain set forth in SEQ ID NO: 98, wherein said polypeptide binds to the E protein of Zika virus.

199 One or more polypeptides comprising an amino acid sequence having at least 95% sequence identity to the immunoglobulin light chain set forth in SEQ ID NO: 100 and an amino acid sequence having at least 95% sequence identity to the immunoglobulin heavy chain set forth in SEQ ID NO: 98, wherein said one or more polypeptides binds to the E protein of Zika virus.

200. A composition comprising an antibody or binding fragment thereof comprising an immunoglobulin light chain and/or immunoglobulin heavy chain encoded by the polypeptides of any one of embodiments 197-199.

201. A polypeptide that binds to the E protein of Zika virus and comprises three immunoglobulin light chain CDR sequences, one or more of which differs by no more than one amino acid from the CDR1 sequence of SEQ ID NO: 276, CDR2 sequence of GKN, and CDR3 sequence of SEQ ID NO: 278.

202. A polypeptide that binds to the E protein of Zika virus and comprises three immunoglobulin heavy chain CDR sequences, one or more of which differs by no more than one amino acid from the CDR1 sequence of SEQ ID NO: 189, CDR2 sequence of SEQ ID NO: 190, and CDR3 sequence of SEQ ID NO: 191.

203. One or more polypeptides that bind to the E protein of Zika virus and together comprise six immunoglobulin light and heavy chain CDR sequences, one or more of which differs by no more than one amino acid from the CDR1 sequence of SEQ ID NO: 276, CDR2 sequence of GKN, CDR3 sequence of SEQ ID NO: 278, CDR1 sequence of SEQ ID NO: 189, CDR2 sequence of SEQ ID NO: 190, and CDR3 sequence of SEQ ID NO: 191.

204. A composition comprising an antibody or binding fragment thereof comprising immunoglobulin light chain CDRs and/or immunoglobulin heavy chain CDRS encoded by the polypeptides of any one of embodiments 201-203.

205. A polypeptide comprising an amino acid sequence having at least 95% sequence identity to the immunoglobulin light chain set forth in SEQ ID NO: 104, wherein said polypeptide binds to the E protein of Zika virus.

206. A polypeptide comprising an amino acid sequence having at least 95% sequence identity to the immunoglobulin heavy chain set forth in SEQ ID NO: 102, wherein said polypeptide binds to the E protein of Zika virus.

207. One or more polypeptides comprising an amino acid sequence having at least 95% sequence identity to the immunoglobulin light chain set forth in SEQ ID NO: 104 and an amino acid sequence having at least 95% sequence identity to the immunoglobulin heavy chain set forth in SEQ ID NO: 102, wherein said one or more polypeptides binds to the E protein of Zika virus.

208. A composition comprising an antibody or binding fragment thereof comprising an immunoglobulin light chain and/or immunoglobulin heavy chain encoded by the polypeptides of any one of embodiments 205-207.

209. A polypeptide that binds to the E protein of Zika virus and comprises three immunoglobulin light chain CDR sequences, one or more of which differs by no more than one amino acid from the CDR1 sequence of SEQ ID NO: 279, CDR2 sequence of DAS, and CDR3 sequence of SEQ ID NO: 281.

210. A polypeptide that binds to the E protein of Zika virus and comprises three immunoglobulin heavy chain CDR sequences, one or more of which differs by no more than one amino acid from the CDR1 sequence of SEQ ID NO: 192, CDR2 sequence of SEQ ID NO: 193, and CDR3 sequence of SEQ ID NO: 194.

211. One or more polypeptides that bind to the E protein of Zika virus and together comprise six immunoglobulin light and heavy chain CDR sequences, one or more of which differs by no more than one amino acid from the CDR1 sequence of SEQ ID NO: 279, CDR2 sequence of DAS, CDR3 sequence of SEQ ID NO: 281, CDR1 sequence of SEQ ID NO: 192, CDR2 sequence of SEQ ID NO: 193, and CDR3 sequence of SEQ ID NO: 194.

212. A composition comprising an antibody or binding fragment thereof comprising immunoglobulin light chain CDRs and/or immunoglobulin heavy chain CDRS encoded by the polypeptides of any one of embodiments 209-211.

213. A polypeptide comprising an amino acid sequence having at least 95% sequence identity to the immunoglobulin light chain set forth in SEQ ID NO: 108, wherein said polypeptide binds to the E protein of Zika virus.

214. A polypeptide comprising an amino acid sequence having at least 95% sequence identity to the immunoglobulin heavy chain set forth in SEQ ID NO: 106, wherein said polypeptide binds to the E protein of Zika virus.

215. One or more polypeptides comprising an amino acid sequence having at least 95% sequence identity to the immunoglobulin light chain set forth in SEQ ID NO: 108 and an amino acid sequence having at least 95% sequence identity to the immunoglobulin heavy chain set forth in SEQ ID NO: 106, wherein said one or more polypeptides binds to the E protein of Zika virus.

216. A composition comprising an antibody or binding fragment thereof comprising an immunoglobulin light chain and/or immunoglobulin heavy chain encoded by the polypeptides of any one of embodiments 213-215.

217. A polypeptide that binds to the E protein of Zika virus and comprises three immunoglobulin light chain CDR sequences, one or more of which differs by no more than one amino acid from the CDR1 sequence of SEQ ID NO: 285, CDR2 sequence of KAS, and CDR3 sequence of SEQ ID NO: 287.

218. A polypeptide that binds to the E protein of Zika virus and comprises three immunoglobulin heavy chain CDR sequences, one or more of which differs by no more than one amino acid from the CDR1 sequence of SEQ ID NO:201, CDR2 sequence of SEQ ID NO: 202, and CDR3 sequence of SEQ ID NO: 203.

219. One or more polypeptides that bind to the E protein of Zika virus and together comprise six immunoglobulin light and heavy chain CDR sequences, one or more of which differs by no more than one amino acid from the CDR1 sequence of SEQ ID NO: 285, CDR2 sequence of KAS, CDR3 sequence of SEQ ID NO: 287, CDR1 sequence of SEQ ID NO: 201, CDR2 sequence of SEQ ID NO: 202, and CDR3 sequence of SEQ ID NO: 203.

220. A composition comprising an antibody or binding fragment thereof comprising immunoglobulin light chain CDRs and/or immunoglobulin heavy chain CDRS encoded by the polypeptides of any one of embodiments 217-219.

221. A polypeptide comprising an amino acid sequence having at least 95% sequence identity to the immunoglobulin light chain set forth in SEQ ID NO: 112, wherein said polypeptide binds to the E protein of Zika virus.

222. A polypeptide comprising an amino acid sequence having at least 95% sequence identity to the immunoglobulin heavy chain set forth in SEQ ID NO: 114, wherein said polypeptide binds to the E protein of Zika virus.

223. One or more polypeptides comprising an amino acid sequence having at least 95% sequence identity the immunoglobulin light chain set forth in SEQ ID NO: 112 and an amino acid sequence having at least 95% sequence identity to the immunoglobulin heavy chain set forth in SEQ ID NO: 114, wherein said one or more polypeptides binds to the E protein of Zika virus.

224. A composition comprising an antibody or binding fragment thereof comprising an immunoglobulin light chain and/or immunoglobulin heavy chain encoded by the polypeptides of any one of embodiments 221-223.

225. A polypeptide that binds to the E protein of Zika virus and comprises three immunoglobulin light chain CDR sequences, one or more of which differs by no more than one amino acid from the CDR1 sequence of SEQ ID NO: 288, CDR2 sequence of DDR, and CDR3 sequence of SEQ ID NO: 290.

226. A polypeptide that binds to the E protein of Zika virus and comprises three immunoglobulin heavy chain CDR sequences, one or more of which differs by no more than one amino acid from the CDR1 sequence of SEQ ID NO: 201, CDR2 sequence of SEQ ID NO: 202, and CDR3 sequence of SEQ ID NO: 203.

227. One or more polypeptides that bind to the E protein of Zika virus and together comprise six immunoglobulin light and heavy chain CDR sequences, one or more of which differs by no more than one amino acid from the CDR1 sequence of SEQ ID NO: 288, CDR2 sequence of DDR, CDR3 sequence of SEQ ID NO: 290, CDR1 sequence of SEQ ID NO: 201, CDR2 sequence of SEQ ID NO: 202, and CDR3 sequence of SEQ ID NO: 203.

228. A composition comprising an antibody or binding fragment thereof comprising immunoglobulin light chain CDRs and/or immunoglobulin heavy chain CDRS encoded by the polypeptides of any one of embodiments 225-227.

229. A polypeptide comprising an amino acid sequence having at least 95% sequence identity to the immunoglobulin light chain set forth in SEQ ID NO: 116, wherein said polypeptide binds to the E protein of Zika virus.

230. A polypeptide comprising an amino acid sequence having at least 95% sequence identity to the immunoglobulin heavy chain set forth in SEQ ID NO: 114, wherein said polypeptide binds to the E protein of Zika virus.

231. One or more polypeptides comprising an amino acid sequence having at least 95% sequence identity the immunoglobulin light chain set forth in SEQ ID NO: 116 and an amino acid sequence having at least 95% sequence identity to the immunoglobulin heavy chain set forth in SEQ ID NO: 114, wherein said one or more polypeptides binds to the E protein of Zika virus.

232. A composition comprising an antibody or binding fragment thereof comprising an immunoglobulin light chain and/or immunoglobulin heavy chain encoded by the polypeptides of any one of embodiments 229-231.

233. One or more polypeptides comprising an amino acid sequence having at least 95% sequence identity to:
(i) the immunoglobulin light chain set forth in SEQ ID NO: 4 and the immunoglobulin heavy chain set forth in SEQ ID NO: 2,
(ii) the immunoglobulin light chain set forth in SEQ ID NO: 8 and the immunoglobulin heavy chain set forth in SEQ ID NO: 6
(iii) the immunoglobulin light chain set forth in SEQ ID NO: 12 and the immunoglobulin heavy chain set forth in SEQ ID NO: 10, or
(iv) the immunoglobulin light chain set forth in SEQ ID NO: 16 and the immunoglobulin heavy chain set forth in SEQ ID NO: 14.

234. The composition of any one of the previous composition embodiments, wherein the antibody or binding fragment thereof binds and/or neutralizes Zika virus.

235. The composition of any one of the previous composition embodiments, wherein the antibody isotype is IgG1, IgG2, IgG3, IgG4, IgA1, IgA2, or IgM.

236. The composition of any one of the previous composition embodiments, wherein the composition comprises a therapeutic antibody or binding portion thereof.

237. The composition of any one of the previous composition embodiments, wherein the antibody format is an ScFv, heavy chain only, or VHH.

### REFERENCES

1. Fernandez E, Dejnirattisai W, Cao B, Scheaffer S M, Supasa P, Wongwiwat W, Esakky P, Drury A, Mongkolsapaya J, Moley K H, Mysorekar I U, Screaton G R, Diamond M S. 2017. Human antibodies to the dengue virus E-dimer epitope have therapeutic activity against Zika virus infection. Nat Immunol 18:1261-1269.
2. Rouvinski A, Guardado-Calvo P, Barba-Spaeth G, Duquerroy S, Vaney M C, Kikuti C M, Navarro Sanchez M E, Dejnirattisai W, Wongwiwat W, Haouz A, Girard-Blanc C, Petres S, Shepard W E, Despres P, Arenzana-Seisdedos F, Dussart P, Mongkolsapaya J, Screaton G R, Rey F A. 2015. Recognition determinants of broadly neutralizing human antibodies against dengue viruses. Nature 520:109-13.
3. Robbiani D F, Bozzacco L, Keeffe J R, Khouri R, Olsen P C, Gazumyan A, Schaefer-Babajew D, Avila-Rios S, Nogueira L, Patel R, Azzopardi S A, Uhl L F K, Saeed M, Sevilla-Reyes E E, Agudelo M, Yao K H, Golijanin J, Gristick H B, Lee Y E, Hurley A, Caskey M, Pai J, Oliveira T, Wunder E A, Jr., Sacramento G, Nery N, Jr., Orge C, Costa F, Reis M G, Thomas N M, Eisenreich T, Weinberger D M, de Almeida A R P, West A P, Jr., Rice C M, Bjorkman P J, Reyes-Teran G, Ko A I, MacDonald M R, Nussenzweig M C. 2017. Recurrent Potent Human Neutralizing Antibodies to Zika Virus in Brazil and Mexico. Cell 169:597-609 e11.
4. Zhao H, Fernandez E, Dowd K A, Speer S D, Platt D J, Gorman M J, Govero J, Nelson C A, Pierson T C, Diamond M S, Fremont D H. 2016. Structural Basis of Zika Virus-Specific Antibody Protection. Cell 166:1016-1027.
5. Hasan S S, Miller A, Sapparapu G, Fernandez E, Klose T, Long F, Fokine A, Porta J C, Jiang W, Diamond M S, Crowe J E, Jr., Kuhn R J, Rossmann M G. 2017. A human antibody against Zika virus crosslinks the E protein to prevent infection. Nat Commun 8:14722.
6. Sapparapu G, Fernandez E, Kose N, Bin C, Fox J M, Bombardi R G, Zhao H, Nelson C A, Bryan A L, Barnes T, Davidson E, Mysorekar I U, Fremont D H, Doranz B J, Diamond M S, Crowe J E. 2016. Neutralizing human antibodies prevent Zika virus replication and fetal disease in mice. Nature 540:443-447.
7. Abbink P, Larocca R A, De La Barrera R A, Bricault C A, Moseley E T, Boyd M, Kirilova M, Li Z, Ng'ang'a D, Nanayakkara O, Nityanandam R, Mercado N B, Borducchi E N, Agarwal A, Brinkman A L, Cabral C, Chandrashekar A, Giglio P B, Jetton D, Jimenez J, Lee B C, Mojta S, Molloy K, Shetty M, Neubauer G H, Stephenson K E, Peron J P, Zanotto P M, Misamore J, Finneyfrock B, Lewis M G, Alter G, Modjarrad K, Jarman R G, Eckels K H, Michael N L, Thomas S J, Barouch D H. 2016. Protective efficacy of multiple vaccine platforms against Zika virus challenge in rhesus monkeys. Science 353:1129-32.
8 Modjarrad K, Lin L, George S L, Stephenson K E, Eckels K H, De La Barrera R A, Jarman R G, Sondergaard E, Tennant J, Ansel J L, Mills K, Koren M, Robb M L, Barrett J, Thompson J, Kosel A E, Dawson P, Hale A, Tan C S, Walsh S R, Meyer K E, Brien J, Crowell T A, Blazevic A, Mosby K, Larocca R A, Abbink P, Boyd M, Bricault C A, Seaman M S, Basil A, Walsh M, Tonwe V, Hoft D F, Thomas S J, Barouch D H, Michael N L. 2017. Preliminary aggregate safety and immunogenicity results from three trials of a purified inactivated Zika virus vaccine candidate: phase 1, randomised, double-blind, placebo-controlled clinical trials. Lancet doi: 10.1016/S0140-6736 (17) 33106-9.
9. Shan C, Muruato A E, Nunes B T D, Luo H, Xie X, Medeiros D B A, Wakamiya M, Tesh R B, Barrett A D, Wang T, Weaver S C, Vasconcelos P F C, Rossi S L, Shi P Y. 2017. A live-attenuated Zika virus vaccine candidate induces sterilizing immunity in mouse models. Nat Med 23:763-767.
10. Dowd K A, Ko S Y, Morabito K M, Yang E S, Pelc R S, DeMaso C R, Castilho L R, Abbink P, Boyd M, Nityanandam R, Gordon D N, Gallagher J R, Chen X, Todd J P, Tsybovsky Y, Harris A, Huang Y S, Higgs S, Vanlandingham D L, Andersen H, Lewis M G, De La Barrera R, Eckels K H, Jarman R G, Nason M C, Barouch D H, Roederer M., Kong W P, Mascola J R, Pierson T C, Graham B S. 2016. Rapid development of a DNA vaccine for Zika virus. Science 354:237-240.
11. Gaudinski M R, Houser K V, Morabito K M, Hu Z, Yamshchikov G, Rothwell R S, Berkowitz N, Mendoza F, Saunders J G, Novik L, Hendel C S, Holman L A, Gordon I J, Cox J H, Edupuganti S, McArthur M A, Rouphael N G, Lyke K E, Cummings G E, Sitar S, Bailer R T, Foreman B M, Burgomaster K, Pelc R S, Gordon D N, DeMaso C R, Dowd K A, Laurencot C, Schwartz R M, Mascola J R, Graham B S, Pierson T C, Ledgerwood J E, Chen G L, Vrc, teams VRCs. 2017. Safety, tolerability, and immunogenicity of two Zika virus DNA vaccine candidates in healthy adults: randomised, open-label, phase 1 clinical trials. Lancet doi: 10.1016/S0140-6736 (17) 33105-7.
12. Larocca R A, Abbink P, Peron J P, Zanotto P M, Iampietro M J, Badamchi-Zadeh A, Boyd M, Ng'ang'a D, Kirilova M, Nityanandam R, Mercado N B, Li Z, Moseley E T, Bricault C A, Borducchi E N, Giglio P B, Jetton D, Neubauer G, Nkolola J P, Maxfield L F, De La Barrera R A, Jarman R G, Eckels K H, Michael N L, Thomas S J, Barouch D H. 2016. Vaccine protection against Zika virus from Brazil. Nature 536: 474-8.
13. Richner J M, Himansu S, Dowd K A, Butler S L, Salazar V, Fox J M, Julander J G, Tang W W, Shresta S, Pierson T C, Ciaramella G, Diamond M S. 2017. Modified mRNA Vaccines Protect against Zika Virus Infection. Cell 168:1114-1125 e10.
14. Richner J M, Jagger B W, Shan C, Fontes C R, Dowd K A, Cao B, Himansu S, Caine E A, Nunes B T D, Medeiros D B A, Muruato A E, Foreman B M, Luo H, Wang T, Barrett A D, Weaver S C, Vasconcelos P F C, Rossi S L, Ciaramella G, Mysorekar I U, Pierson T C, Shi P Y, Diamond M S. 2017. Vaccine Mediated Protection Against Zika Virus-Induced Congenital Disease. Cell 170:273-283 e12.
15. Stettler K, Beltramello M, Espinosa D A, Graham V, Cassotta A, Bianchi S, Vanzetta F, Minola A, Jaconi S, Mele F, Foglierini M, Pedotti M, Simonelli L, Dowall S, Atkinson B, Percivalle E, Simmons C P, Varani L, Blum J, Baldanti F, Cameroni E, Hewson R, Harris E, Lanzavecchia A, Sallusto F, Corti D. 2016. Specificity, cross-reactivity, and function of antibodies elicited by Zika virus infection. Science 353:823-6.
16. George J, Valiant W G, Mattapallil M J, Walker M, Huang Y S, Vanlandingham D L, Misamore J, Greenhouse J, Weiss D E, Verthelyi D, Higgs S, Andersen H, Lewis M G, Mattapallil J J. 2017. Prior Exposure to Zika Virus Significantly Enhances Peak Dengue-2 Viremia in Rhesus Macaques. Sci Rep 7:10498.
17. Wang Y, Sundling C, Wilson R, O'Dell S, Chen Y, Dai K, Phad G E, Zhu J, Xiao Y, Mascola J R, Karlsson Hedestam G B, Wyatt R T, Li Y. 2016. High-Resolution Longitudinal Study of HIV-1 Env Vaccine-Elicited B Cell Responses to the Virus Primary Receptor Binding Site Reveals Affinity Maturation and Clonal Persistence. J Immunol 196:3729-3743.
18. Wang Y, O'Dell S, Turner H L, Chiang C I, Lei L, Guenaga J, Wilson R, Martinez-Murillo P, Doria-Rose N, Ward A B, Mascola J R, Wyatt R T, Karlsson Hedestam G B, Li Y. 2017. HIV-1 Cross-Reactive Primary Virus Neutralizing Antibody Response Elicited by Immunization in Nonhuman Primates. J Virol 91: e00910-17.
19. Vasilakos J P, Tomai M A. 2013. The use of Toll-like receptor 7/8 agonists as vaccine adjuvants. Expert Rev Vaccines 12:809-819.
20 Tomai M A, Miller R L, Lipson K E, Kieper W C, Zarraga I E, Vasilakos J P. 2007. Resiquimod and other immune response modifiers as vaccine adjuvants. Expert Rev of Vaccines 6:835-847.
21 Tiller T, Meffre E, Yurasov S, Tsuiji M, Nussenzweig M C, Wardemann H. 2008. Efficient generation of monoclonal antibodies from single human B cells by single cell R T-PCR and expression vector cloning. J Immunol Methods 329:112-24.
22. Quisse L H, Gautreau-Rolland L, Devilder M C, Osborn M, Moyon M, Visentin J, Halary F, Bruggemann M, Buelow R, Anegon I, Saulquin X. 2017. Antigen-specific single B cell sorting and expression-cloning from immunoglobulin humanized rats: a rapid and versatile method for the generation of high affinity and discriminative human monoclonal antibodies. BMC Biotechnol 17:3.
23. Osborn M J, Ma B, Avis S, Binnie A, Dilley J, Yang X, Lindquist K, Menoret S, Iscache A L, Quisse L H, Rajpal A, Anegon I, Neuberger M S, Buelow R, Bruggemann M. 2013. High-affinity IgG antibodies develop naturally in Ig-knockout rats carrying germline human IgH/Igkappa/Iglambda loci bearing the rat C H region. J Immunol 190:1481-90.
24 Wang Y, Sundling C, Wilson R, O'Dell S, Chen Y, Dai K, Phad G E, Zhu J, Xiao Y, Mascola J R, Karlsson Hedestam G B, Wyatt R T, Li Y. 2016. High-Resolution Longitudinal Study of HIV-1 Env Vaccine-Elicited B Cell Responses to the Virus Primary Receptor Binding Site Reveals Affinity Maturation and Clonal Persistence. J Immunol 196:3729-3743.
25. Oliphant T, Nybakken G E, Engle M, Xu Q, Nelson C A, Sukupolvi-Petty S, Marri A, Lachmi B E, Olshevsky U, Fremont D H, Pierson T C, Diamond M S. 2006. Antibody recognition and neutralization determinants on domains I and II of West Nile Virus envelope protein. J Virol 80:12149-59.
26 Dunbar J, Krawczyk K, Leem J, Marks C, Nowak J, Regep C, Georges G, Kelm S, Popovic B, Deane C M. 2016. SAbPred: a structure-based antibody prediction server. Nucleic Acids Res 44: W474-8.
27. Pettersen E F, Goddard T D, Huang C C, Couch G S, Greenblatt D M, Meng E C, Ferrin T E. 2004. UCSF Chimera—a visualization system for exploratory research and analysis. J Comput Chem 25:1605-12.
28 Emsley P, Lohkamp B, Scott W G, Cowtan K. 2010. Features and development of Coot. Acta Crystallogr D Biol Crystallogr 66:486-501.
29 Wang R Y, Song Y, Barad B A, Cheng Y, Fraser J S, DiMaio F. 2016. Automated structure refinement of macromolecular assemblies from cryo-EM maps using Rosetta. Elife 5.
30 Pettersen E F, Goddard T D, Huang C C, Meng E C, Couch G S, Croll T I, Morris J H, Ferrin T E. 2021. UCSF ChimeraX: Structure visualization for researchers, educators, and developers. Protein Sci 30:70-82.
31. Krissinel E, Henrick K. 2007. Inference of macromolecular assemblies from crystalline state. J Mol Biol 372:774-97.

1. An antibody or antibody fragment that binds to the E protein of Zika virus, wherein the antibody or antibody fragment comprises:
(a) a heavy chain variable domain comprising heavy chain CDR1, CDR2 and CDR3 regions that are identical to heavy chain CDR1, CDR2 and CDR3 regions indicated in the heavy chain sequences selected from FIG. 6, wherein the CDR1, CDR2 and CDR3 regions are from the same antibody heavy chain; or
(b) a variant of the heavy chain variable domain of (a) that is otherwise identical to said variable domain except for up to five amino acid substitutions in the collective CDRH1, CDRH2 and CDRH3 regions of the variable domain of (a).

2-18. (canceled)

19. The antibody or antibody fragment of claim 1 further comprising:
(c) a light chain variable domain comprising light chain CDR1, CDR2 and CDR3 regions that are identical to light chain CDR1, CDR2 and CDR3 regions indicated in the light chain sequences selected from FIG. 7, wherein the CDR1, CDR2 and CDR3 regions are from the same antibody light chain; or
(d) a variant of the light chain variable domain of (c) that is otherwise identical to said variable domain except for up to five amino acid substitutions in the collective CDRH1, CDRH2 and CDRH3 regions of the variable domain of (c).

20. The antibody or antibody fragment of claim 1 wherein the antibody or antibody fragment comprises:
a heavy chain variable domain comprising an amino acid sequence that is at least 90% (e.g., at least 91%, 92%, 93%, 94%, 95%, 96%, 97%, 98%, 99%, or 100%) identical to the amino acid sequence of the heavy chain variable domain of an antibody selected from FIG. 6.

21. The antibody or antibody fragment of claim 20, wherein the antibody further comprises a light chain variable domain comprising an amino acid sequence that is at least 90% (e.g., at least 91%, 92%, 93%, 94%, 95%, 96%, 97%, 98%, 99%, or 100%) identical to the light chain variable domain of the same antibody, selected from FIG. 7.

22. The antibody or antibody fragment of claim 19, wherein the heavy chain variable domain and the light chain variable domain are present in separate polypeptides.

23. The antibody or antibody fragment of claim 19, wherein the heavy chain variable domain and the light chain variable domain are present in a single polypeptide.

24. The antibody or antibody fragment of claim 19, wherein the antibody is covalently linked to a peptide or non-peptide moiety.

25. The antibody or antibody fragment of claim 24, wherein the non-peptide moiety is a synthetic polymer, a poly(ethylene glycol) polymer, a covalently linked lipid moiety, a fatty acid moiety a polysaccharide moiety or a carbohydrate moiety.

26. The antibody or antibody fragment of claim 25, wherein the peptide moiety is an antibody light chain, an antibody heavy chain, or an antigen-binding fragment of one of the foregoing.

27. The antibody or antibody fragment of claim 19, wherein the antibody is a single chain Fv (scFv) antibody, a heavy chain-only antibody, a VHH, knob or picobody.

28. The antibody or antibody fragment of claim 19, wherein the antibody or antibody binding fragment neutralizes Zika virus.

29. A composition comprising the antibody or antibody fragment of claim 19.

30. A nucleic acid encoding an antibody or antibody fragment of any claim 19, or the complement of said nucleic acid.

31. A composition comprising the nucleic acid of claim 30.

32. A pharmaceutical composition comprising:
a) the antibody or antibody fragment of claim 19; and
b) a pharmaceutically acceptable carrier.

33. A pharmaceutical composition comprising:
a) the nucleic acid of claim 30; and
b) a pharmaceutically acceptable carrier.

34. The pharmaceutical composition of claim 33, wherein the antibody is encapsulated in a liposome.

35. The pharmaceutical composition of claim 34, wherein the nucleic acid is encapsulated in a liposome.
